(19) United States
(12) Patent Application Publication
(10) Pub. No.: US 2013/0202724 A1
(43) Pub. Date: Aug. 8, 2013

(54) SOYBEAN EXTRACTS FOR THE TREATMENT OF HEPATIC DISORDERS

(71) Applicant: Yaron Ilan, Jerusalem (IL)

(72) Inventor: Yaron Ilan, Jerusalem (IL)

(73) Assignee: Hadasit Medical Research Services and Development Ltd., Jerusalem (IL)

(21) Appl. No.: 13/814,702

(22) PCT Filed: Aug. 4, 2011
(86) PCT No.: PCT/IL11/00632

§ 371 Date: Apr. 19, 2013

Related U.S. Application Data
(60) Provisional application No. 61/371,184, filed on Aug. 6, 2010.
(60) Provisional application No. 61/506,107, filed on Jul. 10, 2011.

(51) Int. Cl. A61K 36/48; A61K 31/167

(52) U.S. Cl. CPC A61K 36/48; A61K 31/167
USPC 424/757

(57) ABSTRACT

The invention provides methods and uses of different soybean extracts, for example, enzymatic, hexane, ethanol or aqueous soybean extracts and combinations thereof for the treatment of hepatic disorders, drug induced hepatic injury, the Metabolic Syndrome or an immune-related disorder in a subject in need thereof. The invention further provides pharmaceutical compositions, kits and methods thereof for treating and preventing hepatic disorders.

### REFERENCE TO RELATED APPLICATIONS

This application is a national stage application under 35 USC 371 of International Application No. PCT/IL2011/000632, filed Aug. 4, 2011, which claims the priority of U.S. Provisional Application No. 61/506,107, filed Jul. 10, 2011, and U.S. Provisional Application No. 61/371,184, filed Aug. 6, 2010, the entire contents of which are incorporated herein by reference.

### FIELD OF THE INVENTION

The present invention relates to composition and methods for the prevention and treatment of hepatic disorders. More particularly, the invention relates to soybean extracts or any enzymatically processed product thereof, pharmaceutical compositions, uses, kits and methods thereof for treating and preventing hepatic disorders.

### BACKGROUND OF THE INVENTION

All publications mentioned throughout this application are fully incorporated herein by reference, including all references cited therein.

Immune therapy involves the exposure of components of the immune system to various elements (cytokines, disease associated antigens and natural metabolites) to combat disease processes in which a dysregulated immune response is thought to play a role Immune dysregulation is thought to play a major part in the pathogenesis or disease course of a great number of disease processes, including various neoplastic, inflammatory, autoimmune, infectious and genetic entities. These disorders can be perceived as a dysbalance between pro-inflammatory (Th1) and anti-inflammatory (Th2) cytokines.

Non-alcoholic steatohepatitis (NASH) is a clinico-pathological entity consisting of hepatic fat accumulation, inflammation and fibrosis, and may progress to cirrhosis in 20% of cases, in patients who have no history of alcohol consumption. NASH is common in patients who suffer of other metabolic disturbances, which are suggested to play a contributing role in the pathogenesis of the disorder. These include insulin resistance, obesity-related ATP depletion, increased free-fatty-acid beta peroxidation, iron accumulation, antioxidant depletion, and leptin deficiency.

The immune system and the regulation of adipose tissue metabolism appear to be closely interlinked. Up to fifty percent of cells within adipose tissues are composed of non-adipose cells, including many immunocytes. Most research has been focused on the immunological consequences of morbid obesity Immunological alterations which are known to exist in obese animals and humans include reduced DTH and mitogen-stimulated lymphocyte proliferation responses, impaired phagocyte number and function, attenuation of insulin induced lymphocyte cytotoxicity, and changes in the CD4/CD8 ratio, especially during weight loss attempts.

Adipose cells are known to secrete pro-inflammatory cytokines, including TNF-β and IL6, which are both related to the level of adiposity. Some of these cytokines are considered to have metabolic effects such as insulin resistance mediated by TNF-β and lipoprotein lipase inhibition mediated by IL6. Several recent studies suggest that the immune system may have an important contributory role in the development of obesity. For example, some cytokines are known to act as adipose tissue regulators. These observations, which point to the fact that obese animals and humans may also be suffering of various alterations in the different arms of the immune system, suggest that modulation of the immune system may change some of the pathogenic mechanisms responsible for the development of morbid obesity.

Metabolic syndrome, also called insulin resistance syndrome or syndrome X, is a cluster of risk factors responsible for much cardiovascular disease morbidity, wherein insulin resistance plays the role of the underlying pathophysiological defect. The metabolic syndrome is a precursor to type II diabetes and a strong risk factor for coronary heart disease (CHD) and stroke. Diagnostic criteria for metabolic syndrome according to the WHO include insulin resistance plus two of the following components: abdominal/central obesity, hypertriglyceridemia, low HDL cholesterol, high blood pressure, high fasting glucose, and microalbuminuria.

Drug hepatotoxicity or drug induced liver injury (DILI) is the most common reason of acute liver failure in the United States [Ostapowicz, G. et al. Ann. Intern. Med. 137:947 (2002); Larson, A. M. et al. Hepatology; 42:1364 (2005)]. The liver is one of the main organs responsible for concentrating and metabolizing a major part of drugs and toxins that are introduced into the eukaryotic organism. These compounds are metabolized by a great number of soluble and membrane-bound enzymes, especially those associated to the hepatocyte endoplasmic reticulum. Toxic hepatocellular injury may be divided into two broad groups direct chemical reactions (intrinsic hepatotoxins), and idiosyncratic reactions or immune-mediated hypersensitivity.

Phytoestrogens are chemicals produced by plants and have similar structure to mammalian estrogens. Phytoestrogens are subdivided into three major classifications, i.e., coumestans, lignans and isoflavones.

Femarelle, which is also known as DT56a and Tofupill, is a natural compound that is an enzymatic isolate of soybeans. DT56a is a selective estrogen receptor modulator (SERM) that has been shown to activate estrogen receptors in human cultured female-derived osteoblasts. In vivo experiments have demonstrated that DT56a displays selective estrogenic activity, stimulating creatine kinase (CK) activity in skeletal tissues similarly to estradiol-17β (E2). It increases bone mineral density in post-menopausal women and relieves vasomotor symptoms without affecting sex steroid hormonal levels or endometrial thickness. Thus, DT56a acts as a SERM and its use in postmenopausal women increases bone mineral density without unwanted estrogenic effects. The use of Femarelle for treating a menopausal symptom and bone density disorders is disclosed by WO 2008/004223.

As indicated above, Femarelle is marketed for use in the treatment of menopausal syndrome and bone loss via its effect as an estrogen receptor binder, however, its immune modulatory effects, its hepato-protective effect and effect on the metabolic syndrome shown herein were not previously described.

WO 2007/060652, which is a previous publication by the present inventor, discloses the use of beta-glycolipides in the treatment of immune-related disorders.

The present invention now shows a surprising and clear hepato-protective effect of different soybean extracts, including enzymatic extracts such as Femarelle and extracts thereof, as well as of other aqueous or hexane soybean-derived extracts M1, OS, M0-1, M0-2, and T1, and some combinations thereof, such as M1 and OS. More specifically, these extracts are shown for the first time to be effective in reducing liver inflammation and improving various metabolic indices.

Hence, it is one object of the invention to provide a method for treating or preventing hepatic disorders, drug induced hepatic injuries and the Metabolic Syndrome in subjects in need thereof, by administering to said subjects at last one soybean extract or any composition or mixture comprising the same.

More specifically, the extracts may be Femarelle or its extracts, or other soybean extracts, such as M1, OS, M0-1, M0-2, and T1, or some combinations thereof. These may be used for treating immune-related disorders and for serving as hepato-protective agents.

Another object of the invention is the provision of a method for the treatment or prevention of acute or chronic toxic effect of an analgesic or an antipyretic drug or any type of liver insult selected from infectious metabolic, toxic, immune, or perfusion or blood flow related hepatic injury, in a subject in need thereof. The provided method comprises the administration of a therapeutically effective amount of at least one soybean extract to said subject.

A further object of the invention is the provision of a pharmaceutical composition for treating and preventing said analgesic or an antipyretic drug or any type of liver insult selected from infectious metabolic, toxic, immune, or perfusion or blood flow related hepatic injury in a subject in need thereof.

These and other objects of the invention will become apparent as the description proceeds.

### SUMMARY OF THE INVENTION

In the first aspect, the invention provides a method of treating, ameliorating preventing or delaying the onset of any one of a hepatic disorder, drug induced hepatic injury, the Metabolic Syndrome or an immune-related disorder in a subject in need thereof. The method comprises the step of administering to the subject a therapeutically effective amount of soybean extract or any composition or mixture comprising the same. Additionally, the composition may further comprise at least one pharmaceutically acceptable carrier, diluent, excipient and/or additive.

In the second aspect, the invention is directed to a method of treating, preventing, ameliorating, reducing or delaying the onset of acute or chronic toxic effect of an analgesic or an antipyretic drug or any type of liver insult in a subject in need thereof. The insult may be any one of infectious metabolic, toxic, immune, or perfusion or blood flow related hepatic injury. The method comprises the step of administering a therapeutically effective amount of soybean extract or any composition or mixture comprising the same, before, simultaneously with, after or any combination thereof, administration of the drug to the subject.

In the third aspect, the invention provides a composition comprising a combination of at least two of:

(a) at least one soybean extract;
(b) at least one enzymatic soybean extract;
(c) at least one hexane soybean extract;
(d) at least one aqueous soybean extract; and
(e) at least one additional therapeutic agent.
The composition optionally further comprising at least one pharmaceutically acceptable carrier, diluent, excipient and/or additive.

In another aspect, the invention relate to the use of a therapeutically effective amount of at least one soybean extract or any composition or mixture comprising the same, in the preparation of a pharmaceutical composition. The composition thus prepared is effective for treating, ameliorating preventing or delaying the onset of any one of hepatic disorder, drug induced hepatic injury, the Metabolic Syndrome or an immune-related disorder in a subject in need thereof.

In yet another aspect, the invention is directed to at least one soybean extract or any composition or mixture comprising the same, for use in treating, ameliorating preventing or delaying the onset of any one of hepatic disorder, drug induced hepatic injury, the Metabolic Syndrome or an immune-related disorder in a subject in need thereof.

In a further aspect, the invention provides a kit comprising:

(a) at least two of:
(i) at least one soybean extract;
(ii) at least one enzymatic soybean extract, optionally in a pharmaceutical dosage form;
(iii) at least one hexane soybean extract, optionally in a pharmaceutical dosage form;
(iv) at least one aqueous soybean extract, optionally in a pharmaceutical dosage form; and
(v) at least one additional therapeutic agent, optionally in a pharmaceutical dosage form;
(b) optionally, container means for containing the at least two dosage forms.

Still further, another aspect of the invention is directed to a method for increasing the maximum amount of acetaminophen administered to a subject without exhibiting acetaminophen toxicity. This method comprises administering of an acetaminophen toxicity inhibiting amount of a soybean extract or any composition or mixture comprising the same, before, simultaneously with, after or any combination thereof, administration of the acetaminophen to the subject.

### BRIEF DESCRIPTION OF THE DRAWINGS

FIG. 1A-1B

Oral Femarelle administration prevents immune-mediated hepatic damage

Female C57Bl/6 mice (16 weeks old) were administered Femarelle orally 0.5 hours prior to an intravenous ConA injection (0.5 mg, 20 mg/kg).

FIG. 1A: Alanine transaminase (ALT) was determined 16 hours following ConA administration in mice treated or untreated with Femarelle.

FIG. 1B: Aspartate aminotransferase (AST) was determined 16 hours following ConA administration in mice treated or untreated with Femarelle.

Data are shown as the mean±SD.

Abbreviations: ALT (alanine transaminase); AST (aspartate aminotransferase); Fem. (Femarelle treatment); conc. (concentration).

FIG. 2

Femarelle ameliorates immune-mediated hepatic damage

Three adult male wild-type C57BL-6 (B6) mice groups (10 mice per group) were administered either 1 μg or 53 μg Femarelle, or vehicle 30 min after they had received an injection of ConA (0.5 mg, 20 mg/kg). The mice were sacrificed 17 h later and serum ALT and AST were determined. Data are shown as the mean±SD of 10 mice in each group. Similar results were obtained in three independent experiments.

Abbreviations: ALT (alanine transaminase); AST (aspartate aminotransferase); Cont. (control).

FIG. 3A-3B

Femarelle ameliorates immune-mediated hepatic histopathology

FIG. 3A: Representative H&E histological sections of livers from control, low dose (1 μg) and high dose (53 μg) Femarelle-treated mice. Femarelle or vehicle were administered orally 30 min after they had received an injection of ConA (0.5 mg, 20 mg/kg). Original magnification×200.

FIG. 3B: Representative TUNEL-stained liver sections of mice treated with ConA (ConA plus vehicle or one of two doses of Femarelle were administered). The livers were removed 17 h after the mice were injected i.v. with ConA. After the livers were deparaffinized, apoptotic fluorescent cells were detected using the TUNEL assay. Original magnification×200. Abbreviations: Cont. (control).

FIG. 4A-4B

High and low doses of Femarelle reduced splenic and hepatic Treg populations, but increased the population of NKT cells in the livers of ConA-injected mice

Seventeen hours after ConA-injected mice were sacrificed, splenocytes and hepatic lymphocytes were prepared as described from vehicle or Femarelle-treated mice. One million cells were analyzed for the expression of markers.

FIG. 4A: Splenocytes expressing CD4, CD8, CD25 and FOXP3. The numbers of purified CD8⁺, CD4⁺CD25⁺, CD4⁺CD25⁺Foxp3⁺, CD8⁺CD25⁺ and CD8⁺CD25⁺FOXP3⁺ cells were calculated. *, p<0.05; **, p<0.02 compared with the vehicle-treated group.

FIG. 4B: Hepatic lymphocytes expressing CD4, CD8, CD25, FOXP3, CD3 and NK1.1. The numbers of purified CD8⁺, CD4⁺CD25⁺, CD4⁺CD25⁺Foxp3⁺, CD8⁺CD25⁺ and CD3⁺NK1.1 (NKT) cells were calculated. *, p<0.05; **, p<0.005 compared with the vehicle-treated group.

Data are shown as the mean±SD of 10 mice in each group.

Abbreviations: % Gat. Cel. (% gated cells); Cont. (control)

FIG. 5A-5B

High and low doses of Femarelle reduced the secretion of IFN-γ and IL-10 in ConA-injected mice

One of two doses of Femarelle or vehicle was orally administered to mice 30 min after ConA injection. Serum was obtained 17 h after ConA injection.

FIG. 5A: Serum IFN-γ levels measured by ELISA in mice injected with ConA.

FIG. 5B: Serum and IL-10 levels measured by ELISA in mice injected with ConA.

Data are shown as the mean±SD of 10 mice in each group. Similar results were obtained in three independent experiments. *, p<0.03 compared with the vehicle-treated group.

Abbreviations: Cont. (control); Ser. (serum)

FIG. 6

Femarelle prevents acetaminophen-mediated liver damage

Female C57Bl/6 mice were administered either 1 μg or 56 μg Femarelle orally 2 hours prior to an intravenous acetaminophen injection (400 mg/kg in C:E in water). Mice were sacrificed 20 hours after acetaminophen administration, and serum ALT was determined.

Abbreviations: Fem. (Femarelle treatment); ALT. (alanine aminotransferase).

FIG. 7

Low dose of Femarelle decreased hepatic injury in acetaminophen-challenged mice

Femarelle or vehicle were orally administered to mice 2 h before administration of acetaminophen. Serum ALT and AST levels in the mice were measured 24 h after being gavaged with 400 mg/kg of acetaminophen. Serum was obtained 24 h after acetaminophen administration. Data are shown as the mean±SD of 6 mice in each group.

Abbreviations: ALT (alanine transaminase); AST (aspartate aminotransferase); Cont. (control).

FIG. 8

Amelioration of liver histopathology of Femarelle-treated mice after acetaminophen challenge

Representative histological H&E-stained liver sections of mice treated orally with Femarelle 2-h after acetaminophen challenge (300 mg/kg gavage administration). The mice were sacrificed 24 h later, and their livers were removed. Original magnification×200.

Abbreviations: Cont. (control).

FIG. 9A-9B

High and low doses of Femarelle did not affect Treg population in the spleens, but low Femarelle dose reduced hepatic Treg and NKT populations of acetaminophen-challenged mice

One of two doses of Femarelle or vehicle was orally administered to mice 2 h before administration of acetaminophen. Twenty-four hours after 400 mg/kg of acetaminophen was administered to the mice, the animals were sacrificed.

FIG. 9A: Splenocytes were prepared as described from vehicle- and Femarelle-treated mice. One million cells were analyzed for the expression of CD4, CD8, CD25 and FOXP3. The numbers of purified CD8⁺, CD4⁺CD25⁺, CD4⁺CD25⁺Foxp3⁺, CD8⁺CD25⁺FOXP3⁺ and CD3.NK1.1 cells were calculated.

FIG. 9B: Hepatic lymphocytes were prepared as described from vehicle- and Femarelle-treated mice. One million cells were analyzed for the expression of CD4, CD8, CD25, FOXP3, CD3 and NK1.1. The numbers of purified CD25⁺, CD4⁺CD25⁺, CD4⁺CD25⁺Foxp3⁺, CD8⁺CD25⁺Foxp3⁺ and CD3⁺NK1.1 (NKT) cells were calculated.

Data are shown as the mean±SD of six mice in each group. *, p<0.005; ** p<0.01 compared with the vehicle-treated group.

Abbreviations: Cont. (control); % Gat. Cel. (% gated cells).

FIG. 10A-10B

High dose of Femarelle ameliorated hepatic injury in ob/ob mice

ob/ob mice were administered 1 μg (low dose), 53 μg (high dose) Femarelle or vehicle (control) for 6 weeks. Serum samples were obtained weekly using tail-vein blood.

FIG. 10A: Serum ALT levels of treated mice. *, p<0.02; **, p=0.006 compared with the control group.

FIG. 10B: Serum AST levels of treated mice. *, p<0.05; **, p<0.004; ***, p=0.001 compared with the vehicle-treated group.

Data are shown as the mean±SD of 5 mice in each group.

Abbreviations: ALT (alanine transaminase); AST (aspartate aminotransferase); Cont. (control); W. (week).

FIG. 11

High dose of Femarelle ameliorated glucose intolerance in ob/ob mice

A glucose tolerance test (GTT) was performed during week 5 after an overnight fast. Glucose was administered orally (1.25 g/kg body weight). The serum glucose level was measured using tail vein blood every 15 min for 3 h by a standard glucometer. Data symbols represent average±SD. For each time point, 5 mice were analyzed. *, p<0.02; **, p<0.005 compared with the control group.

Abbreviations: Cont. (control); Glue. (glucose); t. (min) (time (minute)).

FIG. 12A-12B

High and low doses of Femarelle decreased serum lipid concentrations in ob/ob mice

FIG. 12A: Serum total cholesterol levels was determined after the mice were sacrificed.

FIG. 12B: Serum triglyceride of treated ob/ob mice were determined after the mice were sacrificed.

Data are shown as the mean±SD of 5 mice in each group. *, p<0.03; **, p=0.003, ***, p<0.002 compared with the control group.

Abbreviations: Cont. (control); Ser. TG (serum triglycerides); Tot. Cholest. (total cholesterol).

FIG. 13

Femarelle increased Treg population in the spleens of ob/ob mice

Femarelle or vehicle were orally administered to ob/ob mice every day for 6 weeks. Splenocytes were prepared as described from treated mice. One million cells were analyzed for the expression of CD4, CD25 and FOXP3. The numbers of purified CD25⁺, CD4⁺CD25⁺, CD4⁺CD25⁺Foxp3⁺, and CD3.NK1.1 cells were calculated. Data are shown as the mean±SD of 5 mice in each group. *, p<0.03 compared with the control group.

Abbreviations: Cont. (control); % Gat. Cel. (% gated cells).

FIG. 14

High dose of Femarelle ameliorated hepatic injury in HFD mice

Serum ALT levels of treated mice were measured during weeks 1, 5 and 11. Serum was obtained using tail vein blood. Data are shown as the mean±SD of 5 mice in each group.

Abbreviations: ALT (alanine transaminase); Cont. (control), W. (week).

FIG. 15

Low dose of Femarelle reduced hepatic TG content in HFD mice

Livers were harvested immediately after the mice were sacrificed. TGs were extracted from aliquots of snap-frozen livers and then assayed spectrophotometrically. The number of milligrams of TGs in each sample was calculated based on liver mass, and the amounts of TGs are thus expressed in percentages (mg TGs/g liver).

Abbreviations: Liv. TG (liver triglycerides); Cont. (control.

FIG. 16

Femarelle reduced fasting plasma glucose levels in HFD mice

Femarelle or vehicle were administered to HFD mice three times a week for 11 weeks. Fasting blood glucose levels of all treated HFD mice were monitored every two weeks. Glucose levels were measured using tail vein blood by a standard glucometer. Data are shown as the mean±SD of 6 mice in each group. *, p<0.05; **, p<0.02 compared with the control group.

Abbreviations: Fast. Glue. (fasting blood glucose); Cont. (control), W. (week).

FIG. 17A-17B

High dose of Femarelle ameliorated glucose intolerance in HFD mice

FIG. 17A: A GTT performed in HFD week 4, after an overnight fast.

FIG. 17B: A GTT performed in HFD week 8, after an overnight fast.

Glucose was administered orally (1.25 g/kg body weight). Serum glucose levels in tail vein blood were measured every 15 min for 3 h by a standard glucometer. Data symbols represent average±SD. For each time point, 6 mice were analyzed. *, p<0.003; **, p<0.02 compared with the control group.

Abbreviations: Glue. (glucose); Cont. (control), W. (week); t. (min) (t. (min)).

FIG. 18

High and low doses of Femarelle decreased total cholesterol levels of HFD mice in week 9

Total cholesterol levels of treated HFD mice were measured every two weeks. Data are shown as the mean±SD of 6 mice in each group.*, p<0.05; **, p<0.02 compared with the control group.

Abbreviations: Cont. (control), W. (week); Ser. Cholest. (serum cholesterol).

FIG. 19

High and low doses of Femarelle decrease the population of Tregs but increased the population of NKT cells in the spleens of HFD mice

One of two doses of Femarelle or vehicle was orally administered to HFD mice three times a week for 11 weeks. Splenocytes were prepared as described from treated mice. One million cells were analyzed for the expression of CD4, CD25 and FOXP3. The numbers of purified CD25⁺, CD4⁺CD25⁺, CD4⁺CD25⁺Foxp3⁺ and CD3.NK1.1 cells were calculated. Data are shown as the mean±SD of 6 mice in each group. *, p<0.03; **, p<0.007; ***, p<0.0005 compared with the control group.

Abbreviations: Cont. (control); % Gat. Cel. (% gated cells).

FIG. 20A-20B

Effect of different Femarelle extracts on serum IFN-γ in ConA-challenged mice

Four to six 11-12 week-old adult male C57BL-6 mice per experimental group were treated with indicated Femarelle extracts (described in Table 1) per os for three days and challenged with ConA. Mice were sacrificed 14 after ConA injection and serum IFN-γ was determined

FIG. 20A. Serum IFN-γ measured in ConA-challenged C57BL-6 mice treated with different Femarelle extracts.

FIG. 20B. Serum IFN-γ measured in ConA-challenged C57BL-6 mice treated with indicated doses of Extract-2.

Abbreviations: Cont. (control); Ext. (extract) Ser. IFN-γ (serum interferon-γ).

FIG. 21A-21E

Serum ALT and AST activity is reduced in soybean-derived-extract-protected ConA-challenged mice

Four to six 11-12 week-old adult male C57BL-6 mice per experimental group were treated with indicated extracts per os for three days and challenged with ConA. Mice were sacrificed 14 after ConA injection and serum alanine aminotransferase (ALT) and aspartate aminotransferase (AST) were determined.

FIG. 21A: Serum ALT and AST activities measured in ConA-challenged C57BL-6 mice treated with indicated doses of OS or M1.

FIG. 21B: Serum ALT and AST activities measured in ConA-challenged C57BL-6 mice treated with indicated doses of OS and M1 combinations.

FIG. 21C: Serum ALT and AST activities measured in ConA-challenged C57BL-6 mice treated with indicated doses of GC, M1, M-01, M-02 and T-1.

FIG. 21D: Serum ALT and AST activities measured in ConA-challenged C57BL-6 mice treated with indicated doses of GC, F-1 and a combination of M1 and OS.

FIG. 21E: Serum ALT and AST activities measured in ConA-challenged C57BL-6 mice treated with indicated doses of GC, dexamethasone (positive control), M1, OS and combinations of M1 and OS.

Activities are expressed as [units/L].

Abbreviations: ALT (alanine aminotransferase); AST (aspartate aminotransferase); DEX (dexamethasone); GC (glucosylceramide); cont. (control); F-1 (Femarelle).

FIG. 22A-22C

Serum IFN-γ and TNF-α are reduced in soybean-derived-extract-protected ConA-challenged mice

Four to six 11-12 week-old adult male C57BL-6 mice per experimental group were treated with indicated extracts per os for three days and challenged with ConA. Mice were sacrificed 14 after ConA injection and serum IFN-γ was determined by ELISA.

FIG. 22A: Serum IFN-γ measured in ConA-challenged C57BL-6 mice treated with indicated doses of GC, F-1 and a combination of 0.3 μg OS and 0.3 μg M1.

FIG. 22B: Serum IFN-γ measured in ConA-challenged C57BL-6 mice treated with indicated doses of GC, dexamethasone, M1, OS, and combinations of different doses of OS and M1.

FIG. 22C: Serum TNF-α measured in ConA-challenged C57BL-6 mice treated with indicated doses of GC, dexamethasone, M1, OS, and combinations of different doses of OS and M1.

Abbreviations: DEX (dexamethasone); GC (glucosylceramide); Ser. IFN-γ (serum interferon-γ), Ser. TNF-α (serum Tumor necrosis factor-α); cont. (control).

FIG. 23

Mice fed with a HFD and treated with soybean-derived extract gain weight similar to untreated mice

Groups of five 6-7 weeks old male wild-type C57BL-6 (B6) mice were fed a high fat diet for 12 weeks, and either untreated or treated with OS, GC, M1, or combinations of M1 and OS. Weights, measured every two weeks, are shown.

Abbreviations: DDW (double distilled water); Wei. (weight); W. (week); GC (glucosylceramide).

FIG. 24

M1 and OS soybean-derived extract combination inhibits HFD-induced cholesterol increase

Groups of five 6-7 weeks old male wild-type C57BL-6 (B6) mice were fed a high fat diet for 12 weeks, and either untreated or treated with OS, GC, M1, or combinations of M1 and OS. Serum cholesterol was measured every two weeks.

Abbreviations: DDW (double distilled water); Ser. Cholest. (serum cholesterol); W. (week); GC (glucosylceramide).

FIG. 25A-25B

M1 and OS soybean-derived extracts combination inhibits HFD-induced serum and hepatic triglycerides increase

Groups of five 6-7 weeks old male wild-type C57BL-6 (B6) mice were fed a high fat diet for 12 weeks, and either untreated or treated with OS, GC, M1, or combinations of M1 and OS.

FIG. 25A: Values of serum triglycerides, measured every two weeks, are shown.

FIG. 25B: Values of hepatic triglycerides, assayed after sacrifice at week 12, are shown.

Abbreviations: DDW (double distilled water); Ser. TG (serum triglycerides); % Hepat. TG (% hepatic triglycerides); W. (week); GC (glucosylceramide).

FIG. 26A-26D

Soybean-derived-extracts inhibits HFD-induced blood glucose levels and insulin resistance increase

Groups of five 6-7 weeks old male wild-type C57BL-6 (B6) mice were fed a high fat diet for 12 weeks, and either untreated or treated with OS, GC, M1, or combinations of M1 and OS.

FIG. 26A: Fasting glucose levels, measured every two weeks, are shown.

FIG. 26B: Glucose tolerance test taken at week 4 is shown.

FIG. 26C: Glucose tolerance test taken at week 12 is shown.

FIG. 26D: Values of serum insulin, assayed after sacrifice at week 12, are shown.

Abbreviations: DDW (double distilled water); Fast. Gluc. (fasting glucose); Relat. Gluc. (relative glucose); W. (week); Ser. Insul. (serum insulin); min (minutes); GC (glucosylceramide).

FIG. 27

A specific soybean-derived-extracts combination inhibits HFD-induced TNF-a increase

Groups of five 6-7 weeks old male wild-type C57BL-6 (B6) mice were fed a high fat diet for 12 weeks, and either untreated or treated with OS, GC, M1, or combinations of M1 and OS.

Serum TNF-α was assayed after sacrifice on week 12.

Abbreviations: DDW (double distilled water); Ser. TNF-α (serum Tumor necrosis factor-α); GC (glucosylceramide).

FIG. 28A-28C

soybean-derived extracts induce changes in splenic T-cell populations in HFD-challenged mice

Groups of five 6-7 weeks old male wild-type C57BL-6 (B6) mice were fed a high fat diet for 12 weeks, and either untreated or treated with OS, GC, M1, or combinations of M1 and OS.

FIG. 28A: Splenic CD4⁺CD25⁺FOXp3⁺ populations in soybean extract-treated, HFD-fed mice.

FIG. 28B: Splenic CD25⁺ and FOXp3⁺ populations in soybean extract-treated, HFD-fed mice.

FIG. 28C: Splenic CD8⁺CD25⁺FOXp3⁺ and CD3⁺NK1.1 populations in soybean extract-treated, HFD-fed mice.

Abbreviations: DDW (double distilled water); % Gat. Cel. (% gated cells); GC (glucosylceramide).

FIG. 29

Soybean-derived extracts prevent hepatic lipid accumulation in HFD-challenged mice

Groups of five 6-7 weeks old male wild-type C57BL-6 (B6) mice were fed a high fat diet for 12 weeks, and either untreated or treated with OS, GC, M1, or combinations of M1 and OS.

Hematoxylin and eosin (H&E) stained liver sections are shown. Numbers in parentheses indicate number of mice displaying shown phenotype out of the number of mice in the same treated group.

Abbreviations: DDW (double distilled water); GC (glucosylceramide).

### DETAILED DESCRIPTION OF THE INVENTION

Liver damage is associated with different mechanisms in immune-mediated disease, infectious disorders, drug-induced liver injury and insulin resistance. The present invention show that oral administration of the soybean extracts of the invention, specifically, DT56a or the M1, OS, M0-1, M0-2, and T1 extracts, and some combinations thereof, promoted hepatoprotective effects in different animal models.

Oral administration of DT56a (also known as Femarelle™) or M1, OS, M0-1, M0-2, and T1, extracts and some combinations thereof, exerted a beneficial effect on the immune-mediated liver damage induced by ConA, as indicated by decreased levels of ALT and AST liver enzymes, improved histology, and decreased hepatic apoptosis. Serum IFN-γ levels were also significantly decreased after administration of these soybean extracts. Oral administration of DT56a was also shown to alleviate Acetaminophen-induced liver damage. In mice treated with a low dose of DT56a, both ALT and AST serum levels were reduced, and hepatic histology was improved in response to an APAP challenge. The lack of an effect of a high dose of DT56a suggests a dose-dependent role for this compound.

Tregs play important roles in the pathogenesis of liver damage in immune-mediated hepatitis, Acetaminophen-[N-acetyl-p-aminophenol (APAP)]-induced liver injury and NASH. They are also important in the pathogenesis of metabolic syndromes and insulin resistance [Hotamisligil G. S. Int. J. Obes. Relat. Metab. Disord. 27 Suppl 3:S53-S55 (2003); Masson M. J. et al., Hepatology (Baltimore, Md. 48(3):889-897 (2008); Masubuchi Y. et al., Chemico-biological interactions. 179(2-3):273-279 (2009); Jaeschke H., Hepatology (Baltimore, Md. 48(3):699-701 (2008)]. However, their effects may differ in various immune settings. The data of the present invention show that, in the ConA model, the hepatoprotective effect of a low dose of DT56a was associated with decreased populations of CD4⁺CD25⁺ and CD8⁺CD25⁺ cells, whereas the effect of a high dose was associated with a decreased population of CD4⁺CD25⁺FOXP3⁺ cells. Similarly, in the APAP-induced liver damage model, oral administration of a low dose of DT56a caused a decrease in the population of CD25⁺ and CD4⁺CD25⁺ cells.

Two animal models were used for the assessment of the effect of different soybean extracts such as DT56a or M1, OS, M0-1, M0-2, and T1, extracts on the liver damage associated with insulin resistance. In ob/ob mice, treatment with DT56a led to a significant decrease in ALT and AST serum levels and improved insulin resistance, as demonstrated by a reduction in elevated fasting blood glucose levels and by the GTT. The improved insulin resistance was associated with reductions in serum cholesterol and triglycerides levels. Similarly, in the HFD model, a low dose of DT56a led to decreased serum ALT and hepatic triglycerides levels. Oral DT56a also improved insulin resistance in the HFD model, as indicated by a decrease in fasting blood glucose levels and the GTT. A significant decrease in cholesterol levels was also noted. The beneficial effects of DT56a in both models were independent of changes in body weight.

Analogous results were obtained in HFD-fed mice treated with M1, OS, M0-1, M0-2, and T1 soybean extracts or their combinations. No difference was detected in body weight gain due to HFD between control mice to soybean-treated mice, however some extracts or combinations lowered HFD-induced cholesterol (FIG. 24), serum and hepatic TG (FIGS. 25A and 25B, respectively), fasting glucose level (FIG. 26A) and fasting insulin levels (FIG. 26D), while improving glucose tolerance (FIGS. 26B and 26C). Anti inflammatory and immunomodulatory effects were also observed; HFD-induced serum TNF-α increase was inhibited (FIG. 27), and the HFD-induced increase in splenic regulatory CD4⁺CD25⁺FOXp3⁺ T cell population was inhibited as well, as depicted in FIG. 28A. This inhibition also occurred in splenic CD25⁺ and FOXp3⁺ populations, separately (FIG. 28B). FIG. 28C shows the inhibition of HFD-induced splenic CD8⁺CD25⁺FOXp3⁺ and CD3⁺NK1.1 populations increase by M1 and both M1/OS mixture doses. An improvement in liver histology was also evident in soybean-extract treated HFD mice, as shown in FIG. 29.

Tregs were previously shown to alleviate insulin resistance. In the ob/ob model, the beneficial effect of DT56a was associated with an increased population of CD25⁺, CD4⁺CD25⁺, and CD4⁺CD25⁺FOXP3⁺ cells. These data suggest that, in the ob/ob model, DT56a promotes the redistribution of regulatory cells and alleviates insulin resistance and the associated liver damage. In the HFD model, a decrease in CD25⁺, CD4⁺CD25⁺, and CD4⁺CD25⁺Foxp3 cells was also noted, suggesting that different immune mechanisms may be involved in the two models.

A wide variety of other immune cells is present in normal livers and spleens and may also play roles in inflammation-induced insulin resistance. NKT cells are enriched in the normal mouse liver, and their numbers decrease in ob/ob and HFD models of obesity. In the present invention, the number of NKT cells was increased in the spleens of DT56a-treated insulin-resistant animals, but decreased in the spleens of HFD mice treated with some other soybean extracts, such as M1, and both 0.3 μg and 3 μM1/OS mixture doses.

Oral administration of DT56a exerts a hepatoprotective effect that is independent of the immune background or mechanism of liver damage. The data presented by the invention suggest that DT56a alters the distribution of Tregs in the liver and spleen in different ways, thereby exerting immune-modulatory effects in the various animal models. In some models, this modulatory effect may involve the promotion of anti-inflammatory suppressor cells, whereas in others it may be associated with other mechanisms.

The DT56a soybean derived compound is based on freeze-derived materials that do not include active proteins or glycosphingolipids. The noted effect of DT56a on the immune system may be associated with the presentation of the antigenic factors of soy-derived molecules that can bind and alter Treg function.

As an immune-modulatory effect was noted following oral administration of DT56a, a gut-associated mechanism has also been suggested. The inventor hypothesizes that the antigenic parts of the DT56a—associated molecules induce both professional and non-professional antigen presenting cells in the gut, inducing a signal for the alteration of the inflammatory immune response systemically.

The data on the effect of DT56a on metabolic pathways do not rule out a primary metabolic effect followed by a secondary effect on the immune system. However, in light of recent data suggesting a role for immune-modulatory agents in models of insulin resistance and NASH and the data presented by the invention, growing evidence supports a primary effect of DT56a on the immune system.

Previous studies with DT56a demonstrated its effect as a SERM, enabling the reversal of bone resorption in postmenopausal women and the relief of vasomotor symptoms. Its immune-modulatory role was not previously described. The immune system plays a role in bone resorption and in vasomotor symptoms, suggesting that the noted effect of DT56a on bone and vascular tissues may not be solely due to its role as a SERM. As DT56a has no effect on sex steroid hormonal levels or endometrial thickness, its effect on increasing bone mineral density in postmenopausal women and relieving vasomotor symptoms may thus be related to its immune-modulatory role. The finding that pharmacological doses of DT56a have no effect on the MCF-7 human breast cancer cell line further supports this hypothesis.

In summary, the data presented by the invention suggest that oral administration of different soybean extracts such as the enzymatic extract DT56a (Femarelle) or the aqueous or hexane extracts of soybean, M1, OS, M0-1, M0-2, and T1, that exert a hepatoprotective effect in different animal models that have different alterations of their immune system. The beneficial effect of the different soybean extracts of the invention in insulin resistance-mediated diseases suggests that it may also have a metabolic effect via direct or indirect effects on the immune system.

Thus, in the first aspect, the invention provides a method of treating, ameliorating preventing or delaying the onset of any one of a hepatic disorder, drug induced hepatic injury, the Metabolic Syndrome or an immune-related disorder in a subject in need thereof. The method comprises the step of administering to the subject a therapeutically effective amount of soybean extract or any products or derivatives thereof. Alternatively, any composition or mixture comprising the same may be administered. Additionally, the composition may further comprise at least one pharmaceutically acceptable carrier, diluent, excipient and/or additive.

With respect to the at least one soybean extract, it is appreciated that, according to some embodiments of the method of the invention, it may be any one of an enzymatic soybean extract, a hexane extract and an aqueous extract.

The term “extract” refers to any substances obtained by extracting soy beans using either enzymatic extracts, organic solvents or by hydrophilic solvents. More specifically, the term “extract” refers to any substances obtained by extracting soy beans using either organic solvents such as, for example, hexane, ethyl-acetate or isopropyl-alcohol, or by hydrophilic solvents, such as water. The extracts may be dried after said extraction and may be further extracts by any extraction method, independently from previous extraction steps. Such steps may be repeated independently. Furthermore, other extraction techniques may be employed, non-limiting examples of which include chromatography, including size-exclusion, hydrophobic interaction, and anion and cation exchangers, differential centrifugation, differential precipitation (for example, using ammonium sulfate), differential filtration and dialysis.

Many extraction methods may be used for producing the soybean extracts of the invention.

For example, at least one of an aliphatic organic solvent and water, or supercritical carbon dioxide gas may be used as an extractant for extraction of phospholipids from the soybean, preferably a defatted soybean material. The aliphatic organic solvent is preferably a saturated hydrocarbon, an alcohol, a mixed solvent of saturated hydrocarbon and alcohol, or a mixed solvent of halogenated hydrocarbon and alcohol. It is preferable that the extract be at least one of hexane, ethanol, methanol, hydrous ethanol, isopropyl alcohol, acetonitrile and acetone.

The extract may be enriched with aromatic chromophore containing compounds including the isoflavones genistein, daidzein, formononetin and biochanin and/or their glycosides, and for administration it is generally provided in association with one or more pharmaceutically acceptable carriers, excipients, auxiliaries, and/or diluents.

Plant material may be dried, and may be chopped or otherwise comminuted by methods well known in the art prior to an extract being prepared thereof. The extract may be made from any part of the soy plant, such as roots, bulbs, corms, tubers, leaves, cuttings, flowers, stems, fruits and seeds. More specifically, the extract may be prepared from soy beans.

As well known to those skilled in the art, for enrichment or isolation of aromatic chromophore containing soybean derived compounds, a solvent having a ratio of water to organic solvent in the general order of 0.5% to 70% v/v water:organic solvent, preferably from 1% to 50% organic solvent, may be appropriate. The organic solvent is preferably a C14 organic solvent (such as methanol, ethanol, propanol, propylene glycol, erythrite, butanol, butanediol, acetonitrile, ethyleneglycol, glycidol, glycerol dihydroxyacetone or acetone). Most specifically, the solvent used is either hexane or ethanol.

The extract in this regard is prepared by exposing the plant material to the water/organic solvent mix. The exposure time in general terms is indirectly proportional to the temperature of the mixture. The temperature of the mix may range, for example, from an ambient temperature to boiling temperature. More specifically, the temperature may be between about 10° C. to about 20° C., about 20° C. to about 30° C., about 30° C. to about 40° C., about 40° C. to about 50° C., about 50° C. to about 60° C., about 60° C. to about 70° C., about 70° C. to about 80° C., about 80° C. to about 90° C., or about 90° C. to about 100° C. Exposure time may range between one hour to several weeks. More specifically, the exposure time may be at least 1 hour, at least 2 hours, at least 4 hours, at least 10 hours, at least 24 hours, at least 2 days, at least 4 days, at least 1 week, at least 2 weeks, at least 1 month, or even more. One convenient and non-limiting extraction period is twenty four hours at 90° C. The extract is separated from undissolved plant material and the solvent removed by distillation, rotary evaporation, or other standard procedures for solvent removal. Other fractions, such as the distillation residues containing water soluble and non-water soluble components and water, are preferably extracted with non-water miscible organic solvent or non-polar solvent (such as petroleum ether, pentane, hexane, heptane, octane, benzene or toluene) and the aqueous phase discarded.

Compounds having aromatic chromophore content including the isoflavones genistein, daidzein, formononetin, biochanin flavones including pratensin, reversitrol and Vitamin A may be either removed or enriched or isolated to give a final plant extract as utilized herein by standard procedures. Examples include chromotagraphic techniques, such as preparative high performance liquid chromatography (HPLC) using UV detection, and reverse phase HPLC using UV detection. Aromatic chromophore containing compounds show a characteristic UV absorbence between about 254 and 300 nm as does Vitamin A. This allows these compounds to be readily detected and isolated or removed.

The eluates resulting from the above techniques may be concentrated, (for example, by solvent removal and drying to give a powder), optionally with subsequent formulation into pharmaceutically acceptable compositions.

Examples of chromatographic media include inorganic materials (such as porous silica, controlled poreglass hydroxy apatite, fluorapatite, aluminium oxide), composite materials (such as coated silica, coated polystyrene) and synthetic polymers (polyacrylamide, polymethacrylate, and polystyrene) and reverse phase HPLC matrixes including C8-C18 columns The solvent phase for chromotographic separation may be an organic solvent such as methanol, ethanol, propanol, butanol, pentanol, acetone, acetonitrile, butanone, chloroform, dichloromethane, dichloroethane, dichlorobutane, ethylacetate, ether or dimethyl sulphoxide, which may be used to dissolve the extract prior to separation.

Other procedures for specifically enriching or removing soybean isoflavones include differential extraction with organic solvents, based on the differing solubility of aromatic chromophore containing compounds in certain organic solvents.

As described in the art, extraction of soybeans may also incorporate enzymatic treatment of said soybeans, whether before, during or after mechanical disruption and/or chemical extraction of said soybeans. Therefore, enzymatic treatment of the plant material is specifically contemplated herein. Enzymes used for said extraction include cellulase, hemicellulase, pectinase, protease and other carbohydrases. The use of enzymatic treatment may be carried out under various moisture and temperature conditions suitable for optimal enzyme activity as known in the art. When performing enzymatic treatment of the plant material during chemical extraction, it is appreciated that the solvent and conditions used must be compatible with the maintenance of adequate enzymatic activity, and care must be taken not to inhibit the enzyme activity or to denature it.

According to specific embodiments, the soybeans are ground and extracted using hexane or ethanol. For instance, the ground soybeans may be incubated in the solvent in temperatures ranging from ambient temperatures to 90° C., for a period ranging from 1 hour to three weeks. The extract may then be filtered to remove insoluble material, and the extract is dried using rotary evaporation. Finally, the extract is reconstituted in an appropriate vehicle such as a Cremophor: Ethanol (C:E) mixture as described in the Examples, and optionally, the appropriate carriers diluents and excipients are supplemented to produce a pharmaceutical composition.

Furthermore, in some specific embodiments, the enzymatic soybean extract used by the method of the invention comprises soybean isoflavones. The term “isoflavones” means 3-phenylchromones, isomeric forms of flavones in which the benzene group is attached to the 3 position of the benzopyran ring instead of the 2 position, and their respective metabolites. Whenever the term “isoflavones” is used herein, it is intended to encompass derivatives and metabolites of isoflavones, with particular examples of isoflavone derivatives as described herein. Isoflavones may be found in a number of sources, including, but not limited to, soy. Non-limiting examples of isoflavones include daidzein, 6-O-malonyl daidzein, 6-O-acetyl daidzein, genistein, 6-O-malonyl genistein, 6-O-acetyl genistein, glycitein, 6-O-malonyl glycitein, 6-O-acetyl glycitein, biochanin A, formononetin, or any metabolites of isoflavones.

In yet another specific embodiment, the method of the invention uses Femarelle (DT56a) or any extract thereof, specifically, ethanol extract, as a soybean extracts. In specific embodiments, Femarelle may be considered as an enzymatically processed product of soybean. Femarelle (interchangeably referred to herein also as DT56a and Tofupill), as used herein, may refer to one or more compounds which may be produced from soybean. Femarelle may include one or more phytoestrogen ingredients. Femarelle may include one or more isoflavones which is a main subclass of phytoestrogen. In certain specific embodiments, Femarelle, as used by the present invention is also known as a tofu extract comprising 322 mg DT56a and 108 mg Linum usitatissimum. Such preparation optionally further comprises a pharmaceutically acceptable carrier and/or excipient, such as 100 mg Gelatin.

As mentioned above, the method of the invention may use extracts of Femarelle. According to specific embodiments, ethanol Femarelle (DT56a) extract is prepared by extracting Femarelle using a ratio of about 1 gr of Femarelle per 8.33 ml ethanol, optionally using ultrasonication and overnight incubation of the sonicated extract. The extract may further be filtered and/or evaporated. The product of this process may be reconstituted in various vehicles, such as C:E—Cremophor:Ethanol (C:E) in 1:1 ratio (v/v). As shown by the Examples, Femarelle extract indicated as Extract-2 showed the best hepato-protective effect. Therefore, in certain embodiments, the invention provides methods using extract-2 of Femarelle, for treating hepatic disorders.

In yet an alternative embodiment, the methods of the invention contemplates the use of at least one soybean extract such as any one of a hexane extract and an aqueous extract.

In more specific embodiment, the method of the invention uses at least one soybean extract that may be a hexane extract.

In other specific embodiment, the method of the invention uses an aqueous extract of soybean.

In yet more specific embodiments of the method of the invention, the at least one soybean extract is selected from the group consisting of M1, OS, M-01, M-02 and T1, or any derivative, or any mixture or combination thereof.

More specifically, according to one embodiment, the method of the invention uses at least one hexane extract of soybean, for example, an extract indicated herein as the OS extract.

In yet another specific embodiment, the method of the invention uses at least one aqueous extract of soybean, for example, an extract indicated herein as the M1 extract.

Still further, according to certain specific embodiments the method of the invention may use a combination of both the M1 and OS extracts for treating and preventing said hepatic immune-related and metabolic disorders. The different soybean extracts of the invention, for example the OS and the M1 extracts may be combined at any quantitative ratio of between about 1:1 to 1000:1. It should be appreciated that any quantitative ratio of the combined compounds may be used. As a non-limiting example, a quantitative ratio used between any of the compounds may be: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1500, 1:750, 1:1000. It should be further noted that where the combination of the invention comprises more than two compounds, the quantitative ratio used may be for example, 1:1:1, 1:2:3, 1:10:100, 1:10:100:1000 etc.

It should be appreciated that a therapeutically effective amount of the soybean extract of the invention, specifically, any enzymatic, hexane, ethanol or aqueous soybean extract depends on the mode of delivery, and the condition to be treated.

The inventors demonstrate throughout the provided Examples a variety of clinical and biochemical indices improved by the administration of the soybean extracts of the invention, specifically, the Femarelle or the hexane or aqueous extracts, and composition thereof as well as by treatment according to the methods of the invention. Thus, according to certain embodiments, the methods of the invention lead to at least one of decrease in the plasma level of alanine aminotransferase (ALT), decrease in the plasma level of aspartate aminotransferase (AST), decrease in the plasma level of IFN-γ, decrease in the plasma level of TNF-α, decrease in the plasma level of total cholesterol, decrease in the plasma level of triglycerides, decrease in the fasting plasma level of glucose, decrease in insulin resistance, decrease in hepatic apoptosis, decrease in hepatic necrosis, decrease in hepatic lipid accumulation and modulation of the distribution of at least one of Tregs and NK T cells in a subject in need thereof.

Generally, when used, the terms increase, elevate or augment relate to the induction of an increase, elevation or augmentation in a value, a process, a phenomenon or a phenotype referred to, such as for example, serum levels of certain compounds. Said increase, elevation or augmentation may also be by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.

Converesly, the terms “inhibit”, “inhibition”, “reduce” and “reduction” as used herein, means the restriction, retardation, reduction, decrease or diminishing of a value, a process, a phenomenon or a phenotype by at least about 1%-100%. Said restriction, retardation, reduction, decrease or diminishing of a process, a phenomenon or a phenotype may also be by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.

More specifically, in certain embodiment, the decrease in the plasma level of alanine aminotransferase (ALT) caused by the use of the soybean extracts of the invention, may be of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.

The decrease in the plasma level of aspartate aminotransferase (AST) caused by the use of the soybean extracts of the invention, may be of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.

The decrease in the plasma level of IFN-γ caused by treatment with the soybean extracts of the invention may be of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.

In yet another embodiment, the decrease in the plasma level of TNF-αcaused by the use of the soybean extracts of the invention, may be of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.

Still further, in certain embodiments, the decrease in the plasma level of total cholesterol caused by the use of the soybean extracts of the invention, may be of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.

The decrease in the plasma level of triglycerides caused by the use of the soybean extracts of the invention, may be of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.

The decrease in the fasting plasma level of glucose caused by the use of the soybean extracts of the invention, may be of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.

The decrease in the insulin resistance caused by the use of the soybean extracts of the invention, may be of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.

Thus, the increase in the sensitivity to insulin caused by the use of the soybean extracts of the invention, may be of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.

The decrease in the hepatic apoptosis caused by the use of the soybean extracts of the invention, may be of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%, as judged by % apoptotic cells in any given liver section, for example.

The decrease in the hepatic necrosis caused by the use of the soybean extracts of the invention, may be of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%, as judged by % necrotic cells in any given liver section, for example.

The decrease in the hepatic lipid accumulation caused by the use of the soybean extracts of the invention, may be of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%, as judged by % area taken by adipose cells in any given liver section, for example.

The modulation of the distribution of at least one of Tregs and NK T cells caused by the use of the soybean extracts of the invention, may be of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% increase or decrease for any specific organ or tissue, as judged by % Tregs and/or NK T cells counted in a FACS assay, for example.

As shown by Examples, the soybean extracts of the invention as well as combined compositions thereof that will be described herein after, exhibit a clear immuno-modulatory effect on immune-related cell. An immune-related cell may be Treg cell, an APC (such as DC) or any other cell associated directly or indirectly with the immune system including but not limited to platelets, macrophages, any type of B cell, T cell (including double negative cells), and any type of non-professional antigen presenting cell, adipocytes, endothelial cell, any type of cell that is part of an organ, specifically, an organ connected to the treated immune-related disorder and any type of cell having regulatory enhancing or suppressing properties. More particularly, the soybean extracts of the invention demonstrate immuno-modulation, specifically, anti-inflammatory effect on immune-related cells such as specific T regulatory cells for example, CD4⁺LAP⁺, adipocytes and Antigen Presenting Cells (APC), such as DC. Therefore, according to one embodiment, the methods, as well as composition of the invention may be used for inducing at least one of T regulatory (Treg) cells, or any cell having regulatory properties, either suppressive or inductive, adipocyte and Antigen Presenting Cells (APC) in a subject suffering from an immune-related disorder. More specifically, immune-related cells induced by the methods and composition of the invention may be any T regulatory cell, for example any one of CD4⁺LAP⁺ T-reg cells, CD4⁺CD25 T-reg cells, CD8⁺CD25 T-reg cells, FoxP3⁺CD4 T-reg cells, CD25 High T-reg cells, CD127 MFI T-reg cells, CD28 MFI T-reg cells, CTLA4-T-reg cells and HLA-DR T-reg cells.

It is understood that one of skill in the art will recognize that other antigen presenting cells, either professional or non-professional may be useful in the invention, such as B cells, whole spleen cells, peripheral blood macrophages, fibroblasts, platelets, adipocytes, endothelial cell or non-fractionated peripheral blood mononuclear cells (PBMC). Therefore, the invention is not limited to the exemplary cell types which are specifically mentioned and exemplified herein.

According to some embodiments, the soybean extracts of the invention or any compositions thereof exhibit an immunomodulatory effect modulating the Th1/Th2, Th3 cell balance or any type of modulation of the immune system in a subject suffering from an immune-related disorder. Thereby, such extracts or compositions thereof may activate or inhibit an immune response specifically directed toward said disorder in the treated subject.

According to another specific embodiment the soybean extracts used by the method of the invention, specifically, Femarelle or M1, OS or combinations thereof, modulate the Th1/Th2, Th3 cell balance toward an anti-inflammatory Th2, Tr1/Th3 immune response in a subject suffering from an immune-related disorder.

Modulation of the Th1/Th2, Th3 balance towards an anti-inflammatory Th2, Tr1/Th3 response may be particularly applicable in immune related disorders having an undesired unbalanced pro-inflammatory Th1 reaction. For example, such immune-related disorders may be Metabolic Syndrome or any of the conditions comprising the same, an autoimmune disease, graft rejection pathology, inflammatory disease, non alcoholic fatty liver disease, hyperlipidemia and atherosclerosis.

More specifically, the inventors demonstrate the clear advantages of using the extracts of the invention for protecting the liver from immune, metabolic and drug related damage. Accordingly, in some embodiments, the method of the invention is particularly suitable for treating or protecting patients from hepatic disorders selected from any one of immune-mediated hepatitis, non alcoholic fatty liver disease and drug induced hepatic injury (DILI).

According to one specific embodiment, the invention provides methods of treating Metabolic Syndrome using any of the soybean extracts of the invention, specifically, any enzymatic, hexane, ethanol or aqueous soybean extract. When referring to the Metabolic Syndrome or any of the conditions comprising the same, treatable according to the methods of the invention, it should be understood that this group of disorders includes at least one of dyslipoproteinemia (hypertriglyceridemia, hypercholesterolemia, low HDL-cholesterol), obesity, NIDDM (non-insulin dependent diabetes mellitus), IGT (impaired glucose tolerance), blood coagulability, blood fibrinolysis defects and hypertension.

More specifically, Metabolic Syndrome is characterized by a group of metabolic risk factors in one person including:

Abdominal obesity (excessive fat tissue in and around the abdomen); Atherogenic dyslipidemia (blood fat disorders—high triglycerides, low HDL cholesterol and high LDL cholesterol—that foster plaque buildups in artery walls); elevated blood pressure; insulin resistance or glucose intolerance; prothrombotic state (e.g., high fibrinogen or plasminogen activator inhibitor-1 in the blood); and pro-inflammatory state (e.g., elevated C-reactive protein in the blood). People with the metabolic syndrome are at increased risk of coronary heart disease and other diseases related to plaque buildups in artery walls (e.g., stroke and peripheral vascular disease) and type 2 diabetes.

As indicated above, metabolic syndrome is a combination of medical disorders that, when occurring together, increase the risk of developing cardiovascular disease and diabetes. Some studies have shown the prevalence in the USA to be an estimated 25% of the population. As indicate herein before, there are many different medical criteria for the syndrome, but in general, it may include one or more of the following abnormal medical parameters: increased central obesity, dyslipidemia (as manifested, for example in high triglyceride levels and/or low HDL-C levels), hypertension, high fasting plasma glucose, microalbuminuria, and high hs-CRP levels.

The exact mechanisms of the complex pathways of metabolic syndrome are not yet completely known. The pathophysiology is extremely complex and has been only partially elucidated. Most patients are older, obese, sedentary, and have a degree of insulin resistance. Stress can also be a contributing factor. The most important factors are weight, genetics, endocrine disorders such as polycystic ovary syndrome in women of reproductive age, aging and sedentary lifestyle, i.e., low physical activity and excess caloric intake. There is debate regarding whether obesity or insulin resistance is the cause of the metabolic syndrome or if they are consequences of a more far-reaching metabolic derangement. A number of markers of systemic inflammation, including C-reactive protein, are often increased, as are fibrinogen, interleukin 6 (IL-6), Tumor necrosis factor-alpha (TNF-α), and others. Some have pointed to a variety of causes including increased uric acid levels caused by dietary fructose. It is common for there to be a development of visceral fat, after which the adipocytes (fat cells) of the visceral fat increase plasma levels of TNF-α and alter levels of a number of other substances (e.g., adiponectin, resistin, PAI-1). TNF-α has been shown not only to cause the production of inflammatory cytokines but possibly to trigger cell signaling by interaction with a TNF-α receptor that may lead to insulin resistance. Chronic inflammation contributes to an increased risk of hypertension, artherosclerosis and diabetes.

It should be therefore appreciated that the method of the invention may be used for the treatment of diabetes. The World Health Organization recognizes three main forms of diabetes mellitus: Type 1, Type 2, and gestational diabetes (occurring during pregnancy), which have different causes and population distributions. While, ultimately, all forms are due to the beta cells of the pancreas being unable to produce sufficient insulin to prevent hyperglycemia, the causes are different. Type 1 diabetes is usually due to autoimmune destruction of the pancreatic beta cells. Type 2 diabetes is characterized by insulin resistance in target tissues, this causes a need for abnormally high amounts of insulin and diabetes develops when the beta cells cannot meet this demand. Gestational diabetes is similar to type 2 diabetes in that it involves insulin resistance, hormones in pregnancy may cause insulin resistance in women genetically predisposed to developing this condition.

Acute complication of diabetes (hypoglycemia, ketoacidosis or nonketotic hyperosmolar coma) may occur if the disease is not adequately controlled. Serious long-term complications include cardiovascular disease (doubled risk), chronic renal failure, retinal damage (which can lead to blindness), nerve damage (of several kinds), and microvascular damage, which may cause impotence and poor healing. Poor healing of wounds, particularly of the feet, can lead to gangrene, which may require amputation.

More specifically, according to one embodiment, the immunomodulatory soybean extracts of the invention may be used for the treatment of Type 1 diabetes. Type 1 diabetes mellitus is characterized by loss of the insulin-producing beta cells of the islets of Langerhans in the pancreas, leading to a deficiency of insulin. The main cause of this beta cell loss is a T-cell mediated autoimmune attack. According to another embodiment, the soybean extracts of the invention is intended for treating type 2 diabetes.

According to one specific embodiment, as also demonstrated by Example 1, the invention provides a method for the treatment, prevention and prophylaxis of immune-mediated hepatitis. Accordingly, in some specific embodiments, the method of the invention is particularly suitable for treating or protecting patients from immune-mediated hepatitis Immune-mediated hepatitis, or autoimmune hepatitis, is a chronic disease, characterized by continuing hepatocellular inflammation and necrosis. Anomalous presentation of human leukocyte antigen (HLA) class II on the surface of hepatocytes, possibly due to genetic predisposition or acute liver infection, causes a cell-mediated immune response against the body's own liver, resulting in autoimmune hepatitis. This abnormal immune response results in inflammation of the liver, which can lead to further complications, including cirrhosis Immune serum markers frequently are present, autoantibodies against liver-specific and non-liver-specific antigens and increased immunoglobulin G (IgG) levels. The disease often is associated with other autoimmune diseases. Autoimmune hepatitis cannot be explained on the basis of chronic viral infection, alcohol consumption, or exposure to hepatotoxic medications or chemicals.

In yet another specific embodiment, the method of the invention is particularly suitable for treating or protecting patients from non alcoholic fatty liver disease. Steatohepatitis, a type of liver disease, characterized by inflammation of the liver with concurrent fat accumulation in liver, is frequently found in people with diabetes and obesity. When not associated with excessive alcohol intake, it is referred to as non-alcoholic steatohepatitis, or NASH and is the progressive form of the relatively benign Non-alcoholic fatty liver disease. NASH may progress to cirrhosis, and is believed to be a frequent cause of unexplained cirrhosis. NASH is also associated with Lysosomal Acid Lipase Deficiency. Steatohepatitis is characterized microscopically by hepatic fat accumulation (steatosis), mixed lobular inflammation, ballooning degeneration of hepatocytes (sometimes with identifiable Mallory bodies), glycogenated hepatocyte nuclei, and pericellular fibrosis. The “chicken wire” pattern of the pericellular fibrosis, which affects portal areas only secondarily in later stages, is very characteristic and is identified on trichrome stains.

Still further, as will be discussed in detail herein after, in certain embodiments, the method of the invention is particularly suitable for treating or protecting patients from drug induced hepatic injury (DILI).

In more specific embodiments, methods using the soybean extracts of the invention as well as combined compositions thereof described herein can also be used to treat or prevent graft rejection in a transplant recipient. For example, the soybean extracts can be used in a wide variety of tissue and organ transplant procedures, e.g., the compositions can be used to induce central tolerance in a recipient of a graft of cells, e.g., stem cells such as bone marrow and/or of a tissue or organ such as pancreatic islets, liver, kidney, heart, lung, skin, muscle, neuronal tissue, stomach, and intestines. Thus, the new methods can be applied in treatments of diseases or conditions that entail cell, tissue or organ transplantation (e.g., liver transplantation to treat hypercholesterolemia, transplantation of muscle cells to treat muscular dystrophy, or transplantation of neuronal tissue to treat Huntington's disease or Parkinson's disease).

The invention is further related to the treatment of diseases that are associated with alteration of the immune balance in any type or form such as, for example, without being limited, chronic liver diseases and Alzheimer disease, hepatic encephalopathy, ADHD, metabolic syndrome, diabetes both type 1 and type 2, atherosclerosis or chronic fatigue syndrome, NASH, obesity, hepatic encephalopathy and potentially several immune mediated disorders among them Alopecia Areata, Lupus, Anlcylosing Spondylitis, Meniere's Disease, Antiphospholipid Syndrome, Mixed Connective Tissue Disease, Autoimmune Addison's Disease, Multiple Sclerosis, Autoimmune Hemolytic Anemia, Myasthenia Gravis, Autoimmune Hepatitis, Pemphigus Vulgaris, Behcet's Disease, Pernicious Anemia, Bullous Pemphigoid, Polyarthritis Nodosa, Cardiomyopathy, Polychondritis, Celiac Sprue-Dermatitis, Polyglandular Syndromes, Chronic Fatigue Syndrome (CFIDS), Polymyalgia Rheumatica, Chronic Inflammatory Demyelinating, Polymyositis and Dermatomyositis, Chronic Inflammatory Polyneuropathy, Primary Agammaglobulinemia, Churg-Strauss Syndrome, Primary Biliary Cirrhosis, Cicatricial Pemphigoid, Psoriasis, CREST Syndrome, Raynaud's Phenomenon, Cold Agglutinin Disease, Reiter's Syndrome, Crohn's Disease, Rheumatic Fever, Discoid Lupus, Rheumatoid Arthritis, Essential Mixed, Cryoglobulinemia Sarcoidosis, Fibromyalgia, Scleroderma, Grave's Disease, Sjogren's Syndrome, Guillain-Barre, Stiff-Man Syndrome, Hashimoto's Thyroiditis, Takayasu Arteritis, Idiopathic Pulmonary Fibrosis, Temporal Arteritis/Giant Cell Arteritis, Idiopathic Thrombocytopenia Purpura (ITP), Ulcerative Colitis, IgA Nephropathy, Uveitis, Insulin Dependent Diabetes (Type I), Vasculitis, Lichen Planus, and Vitiligo. The compositions described herein can be administered to a subject to treat or prevent disorders associated with an abnormal or unwanted immune response associated with cell, tissue or organ transplantation, e.g., renal, hepatic, and cardiac transplantation, e.g., graft versus host disease (GVHD), or to prevent allograft rejection, by the oral, enteral, nasal, topical or mucosal administration of soy derived extracts.

In another alternative and specific embodiment, the soybean extracts of the invention or any composition thereof may modulate the Th1/Th2, Th3 cell balance toward a pro-inflammatory Th1 immune response in a subject suffering from an immune-related disorder.

Modulation of an immune response towards a pro-inflammatory reaction may be applicable in treating conditions where enhancement of an immune response is desired. More specifically, such immune-related disorder may be a malignant and non-malignant proliferative disorder, infectious disease, genetic disease and neurodegenerative disorders.

Thus, according to certain embodiments, the soybean extracts of the invention, specifically, any enzymatic, hexane, ethanol or aqueous soybean extract, more specifically, Femarelle, M1, OS or any combinations thereof, as immunomodulatory agents may be applicable in methods for the treatment of a malignancy. In cancerous situations, modulation of the Th1/Th2, Th3 cell balance may be in the direction of inducing a pro-inflammatory response or in augmenting the anti-tumor associated antigens immunity. As used herein to describe the present invention, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods and soybean extracts of the present invention may be used in the treatment of non-solid and solid tumors.

Malignancy, as contemplated in the present invention may be selected from the group consisting of carcinomas, melanomas, lymphomas, myeloma, leukemia and sarcomas. Malignancies that may find utility in the present invention can comprise but are not limited to hematological malignancies (including leukemia, lymphoma and myeloproliferative disorders), hypoplastic and aplastic anemia (both virally induced and idiopathic), myelodysplastic syndromes, all types of paraneoplastic syndromes (both immune mediated and idiopathic) and solid tumors (including lung, liver, breast, colon, prostate GI tract, pancreas and Karposi). More particularly, the malignant disorder may be hepaotcellular carcinoma, colon cancer, melanoma, myeloma, acute or chronic leukemia.

It should be noted that by inducing a pro-inflammatory response, the immune-modulatory soybean extracts of the invention may be applicable for treating infectious diseases caused by bacterial infections, viral infections, fungal infections, or parasitic infections. More specifically, the viral infection may be caused by any one of HBV, HCV or HIV.

According to another embodiment, soybean extracts of the invention, specifically, any enzymatic, hexane, ethanol or aqueous soybean extract used by the methods of the invention, or the composition or mixture comprising the same, may be suitable for oral, nasal, topical or mucosal administration.

More specifically, it is understood that the methods of the invention involve administering soybean extracts, any combination or mixture thereof or any compositions comprising the same. There are numerous administration routes that may be used. In some embodiments, the administration is at least one of oral, mucosal, nasal, transdermal, pulmonary, buccal or sublingual administration, or any combinations thereof. Other administration modes are also applicable, for example, subcutaneous, rectal, or parenteral (including intramuscular, intraperitoneal (IP), intravenous (IV) and intradermal) administration.

An amount adequate to accomplish this is defined as a “therapeutically effective dose.” Amounts effective for this use will depend upon the severity of the condition and the general state of the patient's own immune system, but generally range from about 0.001 to about 1000 mg/Kg of soybean extract of the invention. Specifically, a soybean extract used may be Femarelle (DT56a) or any ethanol extract thereof, with dosages of from 0.0001 to 5000 mg and 0.01 to 2.5, specifically, 0.001, 0.002, 0.003, 0.004, 0.005, 0.01, 0.02, 0.03, 0.04, 0.05, 0.06, 0.07, 0.08, 0.09, 0.1, 0.2, 0.3 0.4, 0.5, 0.6, 0.7, 0.8, 0.9, 1, 1.5, 2, 2.5, 3, 3.5, 4, 4.5 and 5 mg. More specifically, about 0.005 to 2.5 and most specifically, a low dose of 0.05 mg or a high dose of 2.5 mg Femarelle per Kg of body weight being more commonly used. Single or multiple administrations on a daily, weekly or monthly schedule can be carried out with dose levels and pattern being selected by the treating physician.

In other specific embodiments a therapeutic effective amount of hexane or aqueous soybean extracts used by the method of the invention, specifically, the M1, the OS and any combinations thereof, may range from about 0.0001 to about 5000 mg/Kg, specifically, specifically, about 0.001 to about 1000 mg/Kg, 0.001, 0.002, 0.003, 0.004, 0.005, 0.01, 0.02, 0.03, 0.04, 0.05, 0.06, 0.07, 0.08, 0.09, 0.1, 0.2, 0.3 0.4, 0.5, 0.6, 0.7, 0.8, 0.9, 1, 1.5, 2, 2.5, 3, 3.5, 4, 4.5 and 5 mg. More specifically, about 0.015 to 0.15 and most specifically, a low dose of 0.015 mg or a high dose of 0.15 mg M1, OS or any combinations thereof per Kg of body weight being more commonly used.

It should be appreciated that the effective amount indicated herein may be applicable for any of the methods, compositions, kits and uses described by the invention.

In prophylactic applications, compositions containing the soybean extracts of the invention, or any combination, mixture or cocktail thereof are administered to a patient who is at risk of developing the disease state to enhance the patient's resistance. Such an amount is defined to be a “prophylactically effective dose”. In this use, the precise amounts again depend upon the patient's state of health and general level of immunity, but generally range from 0.0001 to 5000 mg per dose, 0.005 to 100, 0.01 to 100, 0.05 to 10, 5, 1, 0.9, 0.8, 0.7, 0.6, 0.5, 0.4, 0.3, 0.25, 0.2, 0.15, 0.1, especially a low dose of 0.05 mg or a high dose of 2.5 mg Femarelle per Kg of body weight. In other embodiments, a low dose of 0.015 mg or a high dose of 0.15 mg M1, OS or any combinations thereof per Kg of body weight being more commonly used.

Single or multiple administrations of the compositions are administered depending on the dosage and frequency as required and tolerated by the patient. In any event, the composition should provide a sufficient quantity of the soybean extracts of the invention to effectively treat the patient. Preferably, the dosage is administered once but may be applied periodically until either a therapeutic result is achieved or until side effects warrant discontinuation of therapy. Generally, the dose is sufficient to treat or ameliorate symptoms or signs of disease without producing unacceptable toxicity to the patient.

As discussed above, the invention provides different methods of treating, ameliorating preventing or delaying the onset of hepatic disorders in a subject in need. As used herein in the specification and in the claims section below, the term “treat” or “treating” and their derivatives includes substantially inhibiting, slowing or reversing the progression of a condition, substantially ameliorating symptoms of a condition or substantially preventing the appearance of symptoms of a condition, said condition is any one of an immune-related disorder and a hepatic disorder in a subject in need thereof.

The term “prevent” and all variations of this term is intended to mean the countering in advance of pathologic symptoms or a pathologic process progress. In this case it is understood that the composition is applied prior to the observation of clinical symptoms.

The terms “ameliorate” and “amelioration” relate to the improvement in the treated subject condition brought about by the compositions and methods according to the invention, wherein said improvement may be manifested in the forms of inhibition of pathologic processes associated with any one of an immune-related disorder and a hepatic disorder, a significant reduction in their magnitude, or an improvement in a diseased subject physiological state.

It should be further indicated that in certain embodiments where the treated subject is a human or livestock, the term “treat” or “treating” and their derivatives includes substantially inhibiting, slowing or reversing the progression of a condition, substantially ameliorating symptoms of a condition or substantially preventing the appearance of symptoms of a condition according to the invention.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described below.

The terms “delay”, “delaying the onset”, “retard” and all variations thereof are intended to encompass the slowing of the progress and/or exacerbation of an immune-related disorder or a hepatic disorder and their symptoms slowing their progress, further exacerbation or development, so as to appear later than in the absence of the treatment according to the invention.

By “subject in need” or “patient” it is meant any mammal who may be affected by the above-mentioned conditions, and to whom the treatment and diagnosis methods herein described is desired, including human, bovine, equine, canine, murine and feline subjects. Preferably, the patient is a human. Administering of the composition according to the method of the invention to the patient includes both self-administration and administration to the patient by another person.

The invention further encompasses the use of the soybean extracts of the invention for treating any condition related to the conditions descried above. It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology described herein.

N-(4-hydroxyphenyl)ethanamide Paracetamol or acetaminophen is a widely used over-the-counter analgesic (pain reliever) and antipyretic (fever reducer). It is commonly used non-steroidal analgesic agent for the relief of fever, headaches, and other minor aches and pains, and is a major ingredient in numerous cold and flu remedies.

While acetaminophen has fewer gastro-intestinal side effects than aspirin, another commonly used non-steroidal analgesic agent, acute and chronic acetaminophen toxicity can result in gastro-intestinal symptoms, severe liver damage, and even death. The precise intermediates in the acetaminophen toxic metabolite pathway are not yet known. As indicated herein before, it had been thought that when acetaminophen was ingested, the cytochrome P-450 dependent enzyme system of the liver produced a potentially toxic metabolite of acetaminophen which was the cause of acetaminophen toxicity.

It was further believed that when safe amounts of acetaminophen had been ingested, this toxic metabolite was cleared by hepatic glutathione stores. However in the case of acute or chronic overdose, excessive levels of the toxic metabolite were thought to delete the glutathione stores in the liver, resulting in hepatic necrosis. Later studies have proposed that acetaminophen induced hepatic necrosis may be due to cellular oxidative stress, resulting both in lipid peroxidation, protein and non-protein thiol oxidation, and changes in the intracellular calcium homeostasis. Symptoms of acute acetaminophen toxicity are typically mild or non-existent until at least 48 hours post-ingestion.

As shown by Example 2, administration of Femarelle before and after acetaminophen resulted in clear alleviation of drug induced damage. Thus, the invention demonstrate that the use of soybean extracts, specifically, Femarelle (DT56a) serve as a tool for protecting liver from acetaminophen insult due to oxidative stress and dysfunction of innate immune system.

One object of the invention is therefore to provide solutions to the unmet need of DILI by administration of the soybean extracts of the invention, specifically, any enzymatic, hexane, ethanol or aqueous soybean extract, more specifically, Femarelle (DT56a), with the drug, as a preventive composition or alternatively, as a therapeutic composition after DILI is already developed.

It should be noted that the invention further encompasses the use of hexane, ethanol or aqueous soybean extract, more specifically, the M1, OS and combinations thereof, with the drug, as a preventive composition or alternatively, as a therapeutic composition after DILI is already developed.

Thus, the second aspect of the current invention relates to a method of treating, preventing, ameliorating, reducing or delaying the onset of acute or chronic toxic effect of an analgesic or an antipyretic drug or any type of liver insult in a subject in need thereof. Such insult may be any one of infectious metabolic, toxic, immune, or perfusion or blood flow related hepatic injury. The method comprises the step of administering a therapeutically effective amount of soybean extract, or any composition or mixture comprising the same, before, simultaneously with, after or any combination thereof, administration of the drug to the subject.

With respect to the at least one soybean extract used in the method for treating or preventing acute or chronic toxic effect of an analgesic or an antipyretic drug or any type of liver insult, it is appreciated that, according to some embodiments of the method of the invention, it may be any one of an enzymatic soybean extract, a hexane extract and an aqueous extract.

In more specific embodiments of the method for treating or preventing acute or chronic toxic effect of an analgesic or an antipyretic drug or any type of liver insult, when the soybean extract is Femarelle (DT56a) or any extract thereof, specifically, ethanol extract thereof.

In other embodiments, the method of the invention uses at least one hexane extract of soybean, for example, an extract indicated herein as the OS extract. In yet another specific embodiment, the method of the invention uses at least one aqueous extract of soybean, for example, an extract indicated herein as the M1 extract. Still further, according to certain specific embodiments the method of the invention may use a combination of both the M1 and OS extracts.

In a more specific embodiment, said analgesic or antipyretic drug is an inducer or inhibitor of Cytochrom P-450 selected from the group consisting of: Acetaminophen, Phenobarbital, Phenytoin, Carbamazepine, Primidone, Ethanol, Glucocorticoids, Rifampin, Griseofulvin, Quinine, Omeprazole, Amiodarone, Cimetidine, Erythromycin, Grape fruit, Isoniazid, Ketoconazole, Metronidazole, Sulfonamides, Chlorpromazine, phenylbutazone, halogenated anesthetic agents, sulindac, Dapsone, INH, halothane, amoxicillin-clavulanic acid, phenobarbital, Para-amino salicylate, Clofibrate, Procainamide, Gold salts, propylthiouracil, chloramphenicol, nitrofurantoin, methoxyflurane, penicillamine, paraquat, Tetracycline, Contraceptive and anabolic steroids, rifampin, Aspirin and Sodium valproate.

In a particular specific embodiment, said analgesic drug is acetaminophen (paracetamol).

In another embodiment, the method of the invention is specifically applicable in treating and preventing acute or chronic toxic effect such as any one of drug induced liver injury (DILI), drug-induced acute steatosis, cytotoxic hepatocellular injury, acute liver failure (ALF), reperfusion injury, ischemic liver disease and acute cholestatic injury.

In yet another embodiment, any of the soybean extracts of the invention, more specifically, Femarelle (DT56a), or any extracts or derivatives thereof, or the composition or mixture comprising the same, is administered before the administration of acetaminophen to said subject.

According to another embodiment, the soybean extracts of the invention, more specifically, Femarelle (DT56a), or any extracts or derivatives thereof, or the composition or mixture comprising the same, may be administered after the administration of acetaminophen to said subject.

In yet another embodiment, the soybean extracts of the invention, and specifically, Femarelle (DT56a), or any extracts or derivatives thereof, or the composition or mixture comprising the same, may be administered simultaneously with the administration of acetaminophen to said subject.

In a specific embodiment, said simultaneous administration is performed by administering a combined composition comprising the soybean extracts of the invention, specifically, Femarelle (DT56a), or any extracts or derivatives thereof (such as Extract-2), and acetaminophen. The present invention thus provides a novel combination of Femarelle. The invention further provides uses of this novel composition for treating and preventing hepatic disorders, specifically, disorders induced by drugs.

The invention further provides different combinations of soybean extracts, for example, hexane extracts and aqueous extracts, specifically, M1 and OS.

Thus, in the third aspect, the invention provides a composition comprising a combination of at least two of: (a) at least one soybean extract; (b) at least one enzymatic soybean extract; (c) at least one hexane soybean extract; (d) at least one aqueous soybean extract; and (e) at least one additional therapeutic agent. The composition optionally further comprising at least one pharmaceutically acceptable carrier, diluent, excipient and/or additive.

In various embodiments, the composition of the invention comprises a combination of at least one hexane soybean extract and at least one aqueous soybean extract. This composition optionally further comprises an additional therapeutic agent.

As shown by the following Examples, combination of two specific extracts, the M1 and OS, demonstrated the most powerful effect. Thus, in a particular embodiment, the combination of extracts comprises an aqueous soybean extract M1 and a hexane soybean extract OS. It is understood that the different soybean extracts of the invention, for example the OS and the M1 extracts may be combined at any quantitative ratio of between about 1:1 to 1000:1. It should be appreciated that any quantitative ratio of the combined compounds may be used. As a non-limiting example, a quantitative ratio used between any of the compounds may be: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1500, 1:750, 1:1000. It should be further noted that where the combination of the invention comprises more than two compounds, the quantitative ratio used may be for example, 1:1:1, 1:2:3, 1:10:100, 1:10:100:1000 etc.

In some embodiments, the composition of the invention may be a pharmaceutical composition, nutraceutical composition, functional food, functional nutrition product, medical food, medical nutrition product or dietary supplement.

In more specific embodiments, the composition of the invention may be a pharmaceutical composition for treating, ameliorating preventing or delaying the onset of any one of hepatic disorder, drug induced hepatic injury, the Metabolic Syndrome or an immune-related disorder in a subject in need thereof.

As indicated above, the present invention firstly demonstrates reduction of liver injury due to acetaminophen ingestion. The invention further provides the generation of “Safe drug” based on combining of Femarelle with the drug, specifically, acetaminophen. Thus, in another aspect, the invention relates to a pharmaceutical composition for treating, preventing, ameliorating, reducing or delaying the onset of acute or chronic toxic effect of an analgesic or an antipyretic drug or any type of liver insult selected from infectious metabolic, toxic, immune, or perfusion or blood flow related hepatic injury in a subject in need thereof, comprising as an active ingredient a therapeutically effective amount of a combination of soybean extract specifically, Femarelle (DT56a) or any derivatives thereof, and optionally at least one additional therapeutic agent, with a pharmaceutically acceptable carrier.

In another embodiment, the combination of extracts comprises a combination of at least one enzymatic soybean extract and at least one additional therapeutic agent, and the therapeutic agent is an analgesic or antipyretic drug.

In a specific embodiment, said analgesic or antipyretic drug is an inducer or inhibitor of Cytochrom P-450 selected from the group consisting of: Acetaminophen, Phenobarbital, Phenytoin, Carbamazepine, Primidone, Ethanol, Glucocorticoids, Rifampin, Griseofulvin, Quinine, Omeprazole, Amiodarone, Cimetidine, Erythromycin, Grape fruit, Isoniazid, Ketoconazole, Metronidazole, Sulfonamides, Chlorpromazine, phenylbutazone, halogenated anesthetic agents, sulindac, Dapsone, INH, halothane, amoxicillin-clavulanic acid, phenobarbital, Para-amino salicylate, Clofibrate, Procainamide, Gold salts, propylthiouracil, chloramphenicol, nitrofurantoin, methoxyflurane, penicillamine, paraquat, Tetracycline, Contraceptive and anabolic steroids, rifampin, Aspirin and Sodium valproate.

In more specific embodiments, the soybean extract comprised in the composition of the invention is Femarelle (DT56a) or any extract thereof, specifically, ethanol extract thereof, and the analgesic or antipyretic drug is acetaminophen (paracetamol). In certain embodiments, Femarelle may be considered as an enzymetic soybean extract according to the invention.

According to another embodiment, said acute or chronic toxic effect treated by the combined composition of the invention may be any one of drug induced liver injury (DILI), drug-induced acute steatosis, cytotoxic hepatocellular injury, acute liver failure (ALF), reperfusion injury, ischemic liver disease and acute cholestatic injury.

It should be appreciated that the different Cytochrome P-450 inducing or inhibiting drugs may lead to different hepatic injuries, and therefore, may be prevented or treated by the combined compositions of the invention. For example, chlorpromazine, phenylbutazone, halogenated anesthetic agents and sulindac may cause fever, rash and eosinophilia. Dapsone may lead to sulfone syndrome (i.e., fever, rash, anemia, jaundice), Chlorpromazine, erythromycin, amoxicillin- and clavulanic acid may lead to obstructive jaundice. Phenytoin, carbamazepine, Phenobarbital and primidone may cause anticonvulsant hypersensitivity syndrome (i.e., triad of fever, rash, and liver injury), Para-amino salicylate, phenytoin, sulfonamides, may lead to serum sickness syndrome, Clofibrate may lead to Muscular syndrome (i.e., myalgia, stiffness, weakness, elevated creatine kinase level), Procainamide may cause Antinuclear antibodies (ANAs), Gold salts, propylthiouracil, chlorpromazine and chloramphenicol may cause marrow injury. Drugs such as Amiodarone and nitrofurantoin may be lead to associated pulmonary injury and Gold salts, methoxyflurane, penicillamine, paraquat may also lead to Associated renal injury. Tetracycline may cause Fatty liver of pregnancy, Contraceptive and anabolic steroids and rifampin may cause bland jaundice, Aspirin may cause Reye syndrome, Sodium valproate leads to Reyelike syndrome.

Still further, other acute hepatocellular injuries caused by drugs may be treated or prevented by the combined compositions of the invention. For example, acute viral hepatitis-like picture may be caused by INH, halothane, diclofenac and troglitazone, Mononucleosis like picture may be a result of using phenytoin, sulfonamides or dapsone. Chronic hepatocellular injury may be a result of Pemoline or methyldopa. Massive necrosis may be a result of using acetaminophen, halothane or diclofenac. Thus, combining the soybean extracts of the invention with said drugs may be used for preventing and treating such disorders.

Steatosis may also be a result of using different drugs, for example, Macrovesicular steatosis may be caused by alcohol, methotrexate, corticosteroids, minocycline, nifedipine and TPN. Microvesicular steatosis may be caused by alcohol, valproic acid, tetracycline and piroxicam. Steatohepatitis may be a result of Amiodarone, nifedipine, synthetic estrogens and didanosine. Pseudoalcoholic injury may be caused by Amiodarone, Acute cholestasis may be a result of using Amoxicillin-clavulanic acid, erythromycin and sulindac. Chronic cholestasis may be caused by Chlorpromazine, sulfamethoxazole-trimethoprim, tetracycline or ibuprofen. Granulomatous hepatitis may be a result of using Carbamazepine, allopurinol and hydralazine. Vascular injury may be caused by steroids, Neoplasia may be a result of using Contraceptives or anabolic steroids. Adenoma may be caused by steroids, Angiosarcoma may be a result of Vinyl chloride. Hepatocellular carcinoma may be caused by Anabolic steroids, aflatoxin, arsenic or vinyl chloride.

More particularly, a drug such as Amoxicillin may cause a moderate rise in SGOT (serum glutamic oxaloacetic, also known as aspartate transaminase) levels, SGPT (serum glutamic pyruvic transaminase, also known as alanine transaminase) levels, or both, but the significance of this finding is unknown. Hepatic dysfunction caused by this drug including jaundice, hepatic cholestasis, and acute cytolytic hepatitis, have been also reported.

In certain embodiments, the combined compositions of the invention may be applicable for preventing hepatic damage caused by a drug such as amiodarone. This drug may lead to abnormal liver function, as indicated by test results in 15-50% of patients. The spectrum of liver injury is wide, ranging from isolated asymptomatic transaminase elevations to a fulminant disorder. Hepatotoxicity usually develops more than one year after starting therapy, but it can occur in one month. It is usually predictable, dose dependent, and has a direct hepatotoxic effect. Some patients with elevated aminotransferase levels have detectable hepatomegaly, and clinically important liver disease develops in less than 5% of patients. In rare cases, amiodarone toxicity manifests as alcoholic liver disease. Hepatic granulomas are rare Importantly, amiodarone has a very long half-life and therefore may be present in the liver for several months after withdrawal of therapy. Since amiodarone is iodinated, it results in increased density on CT scans, which does not correlate with hepatic injury.

Still further, the combined composition of the invention may also be applicable in cases of using drug such as Chlorpromazine. This drug may lead to liver injury that resembles that of infectious hepatitis with laboratory features of obstructive jaundice rather than those of parenchymal damage. The overall incidence of jaundice is low regardless of dose or indication of the drug. Most cases occur two to four weeks after therapy. Any surgical intervention should be withheld until extrahepatic obstruction is confirmed. It is usually promptly reversible upon withdrawal of the medication; however chronic jaundice has been reported. Chlorpromazine should be administered with caution to persons with liver disease.

A further embodiment of the invention provides the use of the compositions of the invention for preventing or treating liver damage caused by ciprofloxacin. Cholestatic jaundice has been reported with repeated use of quinolones. Approximately 1.9% of patients taking ciprofloxacin show elevated SGPT levels, 1.7% showed elevated SGOT levels, 0.8% have increased alkaline phosphatase levels, and 0.3% showed elevated bilirubin levels. Jaundice is transient, and enzyme levels return to the reference range.

Also a drug such as Diclofenac exhibits variety of potential liver damage that may be treated or prevented by the combined composition of the invention. Elderly females are more susceptible to diclofenac-induced liver injury. Elevations of one or more liver test results may occur. These laboratory abnormalities may progress, may remain unchanged, or may be transient with continued therapy. Borderline or greater elevations of transaminase levels occur in approximately 15% of patients treated with diclofenac. Of the hepatic enzymes, ALT is recommended for monitoring liver injury. Meaningful (>3 times the upper limit of the reference range) elevations of ALT or AST occur in approximately 2% of patients during the first 2 months of treatment. In patients receiving long-term therapy, transaminase levels should be measured periodically within 4-8 weeks of initiating treatment. In addition to the elevation of ALT and AST levels, cases of liver necrosis, jaundice, and fulminant hepatitis with and without jaundice have occurred.

It should be further appreciated that the combined composition of the invention may be used also for preventing or treating liver damage caused by using Erythromycin. This drug may cause hepatic dysfunction, including increased liver enzyme levels and hepatocellular and/or cholestatic hepatitis with or without jaundice. A cholestatic reaction is the most common adverse effect and usually begins within 2-3 weeks of therapy. The liver principally excretes erythromycin; exercise caution when this drug is administered to patients with impaired liver function. The use of erythromycin in patients concurrently taking drugs metabolized by the P-450 system may be associated with elevations in the serum levels of other drugs.

Fluconazole is another example for a drug causing liver damage that may be prevented or treated by the combined use with the soybean extracts of the invention, more specifically, Femarelle (DT56a) used by the invention or any composition comprising the same. The spectrum of hepatic reactions ranges from mild transient elevations in transaminase levels to hepatitis, cholestasis, and fulminant hepatic failure. In fluconazole-associated hepatotoxicity, hepatotoxicity is not obviously related to the total daily dose, duration of therapy, or sex or age of the patient. Fatal reactions occur in patients with serious underlying medical illness.

Fluconazole-associated hepatotoxicity is usually, but not always, reversible upon discontinuation of therapy.

Severe and fatal hepatitis has been reported with Isoniazid (INH) therapy. The risk of developing hepatitis is age related, with an incidence of 8 cases per 1000 persons older than 65 years. In addition, the risk of hepatitis is increased with daily consumption of alcohol. Mild hepatic dysfunction evidenced by a transient elevation of serum transaminase levels occurs in 10-20% of patients taking INH. This abnormality usually appears in the first three months of treatment, but it may occur anytime during therapy. In most instances, enzyme levels return to the reference range, with no need to discontinue the medication. Occasionally, progressive liver damage can occur.

Methyldopa is a further example for a drug causing liver damage that may be prevented by the combined use with Femarelle as described by the invention. Methyldopa is an antihypertensive that is contraindicated in patients with active liver disease. Periodic determination of hepatic function should be performed during the first 6-12 weeks of therapy. Occasionally, fever may occur within 3 weeks of methyldopa therapy, which may be associated with abnormalities in liver function test results or eosinophilia, necessitating discontinuation. In some patients, findings are consistent with those of cholestasis and hepatocellular injury. Rarely, fatal hepatic necrosis has been reported after use of methyldopa, which may represent a hypersensitivity reaction.

Oral contraceptives can lead to intrahepatic cholestasis with pruritus and jaundice in a small number of patients, and therefore may be treated by the combining with Femarelle in a combined preventive composition of the invention. More specifically, patients with recurrent idiopathic jaundice of pregnancy, severe pruritus of pregnancy, or a family history of these disorders are more susceptible to hepatic injury. Oral contraceptives are contraindicated in patients with a history of recurrent jaundice of pregnancy. Benign neoplasms, rarely malignant neoplasm of the liver and hepatic vein occlusion have also been associated with oral contraceptive therapy.

The use of statins/HMG-CoA reductase inhibitors is associated with biochemical abnormalities of liver function, and thus may be also prevented or treated by combined use with the soybean extracts, specifically, Femarelle according to the invention. Moderate elevations of serum transaminase levels (<3 times the upper limit of the reference range) have been reported following initiation of therapy and are often transient. Elevations are not accompanied by any symptoms and do not require interruption of treatment. Persistent increases in serum transaminase levels (>3 times the upper limit of the reference range) occur in approximately 1% of patients, and these patients should be monitored until liver function returns to normal after drug withdrawal. Active liver disease or unexplained transaminase elevations are contraindications to use of these drugs. Exercise caution in patients with a recent history of liver disease or in persons who drink alcohol regularly and in large quantities. Statins are among the most widely prescribed medications in the western world. Currently, 6 statins are available for use in the United States. Due to the information contained in package inserts, physicians tend to be concerned while administering statins to patients with deranged liver function tests. Although no concrete evidence shows that statins cause more harm in patients with elevated liver enzymes (recent data), prescribing them in consultation with a specialist may be prudent.

In certain embodiments, soybean extracts specifically, Femarelle (DT56a) may be also applicable for preventing and treating liver injury caused by Rifampin. The invention thus further provides a combined safe composition of Femarelle and Rifampin that is usually administered with INH. On its own, rifampin may cause mild hepatitis, but this is usually in the context of a general hypersensitivity reaction. Fatalities associated with jaundice have occurred in patients with liver disease and in patients taking rifampin with other hepatotoxic agents.

In yet a further embodiment, the use of soybean extracts of the invention, specifically, Femarelle (DT56a) or combined use thereof may be applicable for preventing or treating liver damage caused by Valproic acid and divalproex sodium. More specifically, Microvesicular steatosis is observed with alcohol, aspirin, valproic acid, amiodarone, piroxicam, stavudine, didanosine, nevirapine, and high doses of tetracycline. Prolonged therapy with methotrexate, INH, ticrynafen, perhexiline, enalapril, and valproic acid may lead to cirrhosis. Valproic acid typically causes microsteatosis. This drug should not be administered to patients with hepatic disease and may be used with caution in patients with a prior history of hepatic disease. Those at particular risk include children younger than 2 years, those with congenital metabolic disorders or organic brain disease, and those with seizure disorders treated with multiple anticonvulsants.

Hepatic failures resulting in fatalities have occurred in patients receiving valproic acid. These incidents usually occur during the first 6 months of treatment and are preceded by nonspecific symptoms such as malaise, weakness, lethargy, facial edema, anorexia, vomiting, and even loss of seizure control. Liver function tests should be performed prior to therapy and at frequent intervals, especially in the first 6 months. Physicians should not rely totally on laboratory results; they should also consider findings from the medical history and physical examination.

It should be further appreciated that the soybean extract or any enzymatically processed product thereof, specifically, Femarelle (DT56a) used by the invention or a combined use thereof may be applicable in preventing or treating liver damage caused by using herbs. In certain embodiments, Femarell may be considered as an enzymatic soybean extract. More specifically as an extract comprising isoflavones and any metabolites thereof. The increasing use of alternative medicines has led to many reports of toxicity. The spectrum of liver disease is wide with these medicines, for example: Senecio/crotalaria (Bush teas) can cause venoocclusive disease germander in teas is used for its anticholinergic and antiseptic properties. Jaundice with high transaminase levels may occur after 2 months of use, but it disappears after stopping the drug. Chaparral is used for a variety of conditions, including weight loss, cancer, and skin conditions. It may cause jaundice and fulminant hepatic failure. Chinese herbs have also been associated with hepatotoxicity.

According to certain embodiments, the use of soybean extracts, specifically, Femarelle (DT56a) or a combined use thereof may be also applicable in treating liver damage caused by recreational drugs. More specifically, Ecstasy is an amphetamine used as a stimulant and may cause hepatitis and cirrhosis. Cocaine abuse has been associated with acute elevation of hepatic enzymes. Liver histology shows necrosis and microvascular changes.

In the third aspect, the invention relate to the use of a therapeutically effective amount of at least one soybean extract, or any composition or mixture comprising the same, in the preparation of a pharmaceutical composition. The composition thus prepared is effective for treating, ameliorating preventing or delaying the onset of any one of hepatic disorder, drug induced hepatic injury, the Metabolic Syndrome or an immune-related disorder in a subject in need thereof.

The inventors also contemplate the use of the invention wherein the at least one soybean extract is any one of an enzymatic soybean extract, a hexane extract and an aqueous extract.

More specifically, the soybean extract may be Femarelle (DT56a) or any ethanol extract thereof. In certain embodiments Femarelle may be considered as enzymatic soybean extract comprising soybean isoflavones.

In alternative embodiments of the use of the invention, the at least one soybean extract is a hexane extract.

In further embodiments of the use of the invention, the at least one soybean extract is an aqueous extract.

In yet further embodiments of the use of the invention, the at least one soybean extract is selected from the group consisting of M1, OS, M-01, M-02 and T1, or any derivative, or any mixture or combination thereof.

According to various embodiments, the composition prepared according to the use of the invention may be suitable for the treatment or prophylaxis of hepatic disorders selected from immune-mediated hepatitis, non alcoholic fatty liver disease and drug induced hepatic injury (DILI).

In certain embodiments, Metabolic Syndrome or any of the conditions comprising the same may be at least one of dyslipoproteinemia (hypertriglyceridemia, hypercholesterolemia, low HDL-cholesterol), obesity, NIDDM (non-insulin dependent diabetes mellitus), IGT (impaired glucose tolerance), blood coagulability, blood fibrinolysis defects and hypertension.

In yet another embodiment, the composition prepared by the use of soybean extracts according to the invention leads to at least one of decrease in the plasma level of alanine aminotransferase (ALT), decrease in the plasma level of aspartate aminotransferase (AST), decrease in the plasma level of IFN-γ, decrease in the plasma level of TNF-α, decrease in the plasma level of total cholesterol, decrease in the plasma level of triglycerides, decrease in the fasting plasma level of glucose, decrease in insulin resistance, decrease in hepatic apoptosis, decrease in hepatic necrosis, decrease in hepatic lipid accumulation and modulation of the distribution of at least one of Tregs and NK T cells in a subject in need thereof.

In another aspect, the invention is directed to at least one soybean extract, any enzymatically processed product or derivatives thereof, or any composition or mixture comprising the same, for use in treating, ameliorating preventing or delaying the onset of any one of hepatic disorder, drug induced hepatic injury, the Metabolic Syndrome or an immune-related disorder in a subject in need thereof.

In one embodiment, the soybean extract of the invention may be any one of an enzymatic soybean extract, a hexane extract and an aqueous extract.

In another specific embodiment, the soybean extract is Femarelle (DT56a) or any ethanol extract thereof.

In yet another specific embodiment, the soybean extract of the invention may be a hexane extract.

In another embodiment, the soybean extract of the invention may be an aqueous extract.

Still further, in certain embodiments, the at least one soybean extract of the invention may be selected from the group consisting of M1, OS, M-01, M-02 and T1, or any derivative, or any mixture or combination thereof.

It should be appreciated that the soybean extracts of the invention are for use in treating hepatic disorder such as immune-mediated hepatitis, non alcoholic fatty liver disease and drug induced hepatic injury (DILI).

In certain embodiments, Metabolic Syndrome or any of the conditions comprising the same may be at least one of dyslipoproteinemia (hypertriglyceridemia, hypercholesterolemia, low HDL-cholesterol), obesity, NIDDM (non-insulin dependent diabetes mellitus), IGT (impaired glucose tolerance), blood coagulability, blood fibrinolysis defects and hypertension.

In yet another embodiment, the soybean extracts according to the invention may lead to at least one of decrease in the plasma level of alanine aminotransferase (ALT), decrease in the plasma level of aspartate aminotransferase (AST), decrease in the plasma level of IFN-γ, decrease in the plasma level of TNF-α, decrease in the plasma level of total cholesterol, decrease in the plasma level of triglycerides, decrease in the fasting plasma level of glucose, decrease in insulin resistance, decrease in hepatic apoptosis, decrease in hepatic necrosis, decrease in hepatic lipid accumulation and modulation of the distribution of at least one of Tregs and NK T cells in a subject in need thereof.

In a further aspect, the invention relates to a pharmaceutical unit dosage form comprising the soybean extracts of the invention or any derivatives thereof, or any composition or mixture comprising the same, and optionally, at least one additional therapeutic agent and a pharmaceutically acceptable carrier, excipient, or diluent.

Excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gum tragacanth or gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidinones, methyl cellulose, pro-gelatinized starch, hydroxypropyl methyl cellulose, microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions and dosage forms of the invention is typically present in from about 50 to about 99 weight percent of the pharmaceutical composition or dosage form.

Disintegrants can be used in the pharmaceutical compositions and oral or mucosal dosage forms of the invention to provide tablets that disintegrate when exposed to an aqueous environment. Tablets containing too much disintegrant might disintegrate in storage, while those containing too little might not disintegrate at a desired rate or under desired conditions.

Thus, a sufficient amount of disintegrant that is neither too much nor too little to detrimentally alter the release of the active ingredients should be used to form the pharmaceutical compositions and solid oral dosage forms described herein. The amount of disintegrant used varies based upon the type of formulation, and is readily discernible to those of ordinary skill in the art.

Disintegrants that can be used in pharmaceutical compositions and oral or mucosal dosage forms of the invention include, but are not limited to, agar-agar, alginic acid, calcium carbonate, Primogel, microcrystalline cellulose, croscarmellose sodium, crospovidone, polacrilin potassium, sodium starch glycolate, corn, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate or Sterotes, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Plano, Tex.), CAB-O-SIL03 (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated. A glidant such as colloidal silicon dioxide can also be used.

The pharmaceutical compositions and oral or mucosal dosage forms provided by the invention can further comprise one or more compounds that reduce the rate by which an active ingredient will decompose. Thus the oral dosage forms described herein can be processed into an immediate release or a sustained release dosage form Immediate release dosage forms may release of the soybean extracts of the invention, for example, Femarelle, M1 OS and any combinations thereof, within a few minutes to within a few hours. Sustained release dosage forms may release of the soybean extracts over a period of several hours, for example, up to 24 hours or longer, if desired. In either case, the delivery can be controlled to be substantially at a certain predetermined rate over the period of delivery. In some embodiments, the solid oral dosage forms can be coated with a polymeric or other known coating material(s) to achieve, for example, greater stability on the shelf or in the gastrointestinal tract, or to achieve control over drug release. Such coating techniques and materials used therein are well-known in the art. Such compounds, which are referred to herein as “stabilizers”, include, but are not limited to, antioxidants such as ascorbic acid and salt buffers. For example, cellulose acetate phthalate, polyvinyl acetate phthalate, hydroxypropylmethyl cellulose phthalate, methacrylic acid-methacrylic acid ester copolymers, cellulose acetate trimellitate, carboxymethylethyl cellulose, and so hydroxypropylmethyl cellulose acetate succinate, among others, can be used to achieve enteric coating. Mixtures of waxes, shellac, rein, ethyl cellulose, acrylic resins, cellulose acetate, silicone elastomers can be used to achieve sustained release coating.

Liquids for oral or mucosal administration represent another convenient dosage form, in which case a solvent can be employed. In some embodiments, the solvent is a buffered liquid such as phosphate buffered saline (PBS). Liquid oral dosage forms can be prepared by combining the active ingredient in a suitable solvent to form a solution, suspension, syrup, or elixir of the active ingredient in the liquid. The solutions, suspensions, syrups, and elixirs may optionally comprise other additives including, but not limited to, glycerin, sorbitol, propylene glycol, sugars or other sweeteners, flavoring agents, and stabilizers. Flavoring agents can include, but are not limited to peppermint, methyl salicylate, or orange flavoring. Sweeteners can include sugars, aspartame, saccharin, sodium cyclamate and xylitol.

In order to reduce the degree of inactivation of orally administered of the soybean extracts of the invention, specifically, any enzymatic, hexane, ethanol or aqueous soybean extract, more specifically, Femarelle (DT56a) or M1, OS and combinations thereof, in the stomach of the treated subject, an antacid can be administered simultaneously with the immunoglobulin, which neutralizes the otherwise acidic character of the gut.

For administration by inhalation of soybean extracts specifically, any enzymatic, hexane, ethanol or aqueous soybean extract, can be delivered in the form of an aerosol spray from pressured container or dispenser which contains a suitable propellant, e.g., a gas such as carbon dioxide, or a nebulizer.

More generally, according to some embodiments, the methods of treatment and prophylaxis of the invention are implemented by administrating the soybean extract nasally using a device selected from the group consisting of: a pump, sprayer, metered device, olfactory delivery device, atomizer or any device adequate to nasal delivery.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

The soybean extract or compositions thereof according to the invention are suitable for nasal administration. The compositions can be administered via an administration device suitable for nasal administration of pharmaceutical compositions. As used herein, an administration device is any pharmaceutically acceptable device adapted to deliver a composition of the invention to a subject's nose. A nasal administration device can be a metered administration device (metered volume, metered dose, or metered-weight) or a continuous (or substantially continuous) aerosol-producing device. Suitable nasal administration devices also include devices that can be adapted or modified for nasal administration. In some embodiments, the nasally administered dose can be absorbed into the bloodstream of a subject.

A metered nasal administration device delivers a fixed (metered) volume or amount (dose) of a nasal composition upon each actuation. Exemplary metered dose devices for nasal administration include, by way of example and without limitation, an atomizer, sprayer, dropper, squeeze tube, squeeze-type spray bottle, pipette, ampule, nasal cannula, metered dose device, nasal spray inhaler, breath actuated bi-directional delivery device, pump spray, pre-compression metered dose spray pump, monospray pump, bispray pump, and pressurized metered dose device. The administration device can be a single-dose disposable device, single-dose reusable device, multi-dose disposable device or multi-dose reusable device.

A continuous aerosol-producing device delivers a mist or aerosol comprising droplet of a nasal composition dispersed in a continuous gas phase (such as air). A nebulizer, pulsating aerosol nebulizer, and a nasal continuous positive air pressure device are exemplary of such a device. Suitable nebulizers include, by way of example and without limitation, an air driven jet nebulizer, ultrasonic nebulizer, capillary nebulizer, electromagnetic nebulizer, pulsating membrane nebulizer, pulsating plate (disc) nebulizer, pulsating/vibrating mesh nebulizer, vibrating plate nebulizer, a nebulizer comprising a vibration generator and an aqueous chamber, a nebulizer comprising a nozzle array, and nebulizers that extrude a liquid formulation through a self-contained nozzle array.

The parameters used to effect nebulization via an electronic nebulizer, such as flow rate, mesh membrane size, aerosol inhalation chamber size, mask size and materials, inlet and outlet valves, outflow tube, internal channel plurality of air outputs communicating with the internal chamber, vibration generator and power source may be varied in accordance with the principles of the present invention to maximize their use with different types of aqueous soybean extract compositions. In some embodiments, substantially all of a dose (weight or volume) is delivered in less than 1.5 minutes or continuously delivered over 1.5 to 60 minutes.

The output rate (the rate at which the dose of the therapeutically effective agent(s) in the soybean extracts solution is administered or delivered) will vary according to the performance parameters of the device used to administer the dose. The higher the output rate of a given device, the lower the amount of time required to deliver or administer the soybean extracts solution, as defined herein.

Nebulizers that nebulize liquid formulations containing no propellant are suitable for use with the compositions provided herein.

The volume or amount of composition administered can vary according to the intended delivery target and administration device used. The amount of active agent in a dose or unit dose can vary according to the intended delivery target and administration device used.

According to particular embodiments, the carrier for nasal administration comprises inactive ingredients selected from the group consisting of glycerol, glycols, preservatives, antioxidants, short chain alcohol, surfactants, lipids, oils, thickeners, pH adjusting agents, chitosan, chitin, osmotic agents, and buffers.

When the active component contained in the soybean extracts of the present invention needs to be stabilized, or when increasing the total volume is required because the amount of the active component is too small to handle correctly, gelatin, gelatin succinate, degradated gelatin, proteins such as human serum albumin, amino acids such as aspartic acid, or sugars such as mannitol may be added to the soybean extracts of the present invention. The methods for adding such agents are not specifically limited, nor are the mixing ratio thereof specifically limited.

To increase both adherence to the nasal mucosa and the stability of the administered drug composition, the present invention may include a water-soluble polymer powder, such as: polyacrylic acid or polymethacrylic acids or metal salts, such as sodium salt or potassium salts, thereof, with a mean particle size of 0.5 to 200 μm, preferably 20 to 100 μm; a water-soluble acrylate polymer such as polyacrylamide, having a molecular weight of 30,000 or greater, preferably 50,000 to 10,000,000; carboxyvinyl polymers, methylcelluloses, ethylcelluloses, hydroxymethylcelluloses, hydroxypropylmethylcelluloses, carboxymethylcelluloses, carboxymethylchitin, polyvinylpyrrolidone, polyvinylalcohols, ester gums, polybutene, synthetic hydroxypropyl-starch, synthetic carboxymethyl-starch, synthetic polyvinylethers, and polyethylene oxide, having an average molecular weight of 20,000 to 9,000,000, and preferably 100,000 to 7,000,000; natural polymers such as hyaluronic acid, sodium alginate, gelatin, gluten, carboxymethyl-starch, hydroxypropyl-starch, gum arabic, mannan, dextran, tragacanth, amylopectin, xanthan gum, locust bean gum, casein, polyvinylethers, and pectin; and mixtures thereof.

Systemic administration can also be by transmucosal means. For transmucosal administration, penetrants appropriate to the barrier to be permeated are used in the formulation. Such penetrants are generally known in the art, and include, for example, for transmucosal administration, detergents, bile salts, and fusidic acid derivatives.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Pharmaceutical formulations adapted for transdermal administration may be presented as discrete patches intended to remain in intimate contact with the epidermis of the recipient for a prolonged period of time.

Pharmaceutical formulations adapted for topical administration may be formulated as ointments, creams, suspensions, lotions, powders, solutions, pastes, gels, sprays, aerosols or oils.

For applications to the eye or other external tissues, for example the mouth and skin, the formulations are preferably applied as a topical ointment or cream. When formulated in an ointment, the active ingredient may be employed with either paraffin or a water-miscible ointment base. Alternatively, the active ingredient may be formulated in a cream with an oil-in-water cream base or a water-in-oil base.

Pharmaceutical formulations adapted for topical administration to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Transmucosal administration can be accomplished through the use of nasal drops or sprays, or rectal or vaginal suppositories.

Soybean extracts according to the invention or any enzymatic, hexane, ethanol or aqueous soybean extract, more specifically, Femarelle (DT56a), M1 or OS can also be prepared in the form of suppositories (e.g., with conventional suppository bases such as cocoa butter and other glycerides) or retention enemas for rectal delivery.

In one embodiment, the oral or mucosal compositions of soybean extracts of the invention are prepared with carriers that will protect the extracts against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or may be obtained commercially.

Dosage, toxicity and therapeutic efficacy of soybean extracts of the invention, or any enzymatically processed product thereof, specifically, isoflavones and any metabolites thereof, more specifically, Femarelle (DT56a) compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between so toxic and therapeutic effects is the therapeutic index and it can be expressed as the ratio LD50/ED50. Compositions which exhibit high therapeutic indices are preferred.

Data obtained from the cell and animal studies can be used in formulating a range of dosage for use in humans. The dosage may vary within this range depending upon the dosage form employed and the route of administration utilized. For any oral or mucosal of soybean extracts of the invention compositions described herein, the therapeutically effective dose can be estimated initially from assays of cell cultures or animal models. A dose may be formulated in animal models to achieve a desired circulating plasma concentration of IL-10, IL-4 or IL-2 and IFN-γ, or of regulatory cells, in the range that includes the IC₅₀ (i.e., the concentration of the test compound which achieves a half-maximal inhibition of symptoms) as determined in cell culture. Such information can be used to more accurately determine useful doses in humans.

Soybean extract or any enzymatically processed product thereof, or any hexane, ethanol or aqueous soybean extract can be administered from one or more times per day to one or more times per week, including once every other day. The oral or mucosal compositions can be administered, e.g., for about 10 to 14 days or longer. The skilled artisan will appreciate that certain factors may influence the dosage and timing required to effectively treat a subject, including but not limited to the severity of the disease or disorder, previous treatments, the general health and/or age of the subject, and other diseases present.

Moreover, treatment of a subject with a therapeutically effective amount of the combined compounds can include a single treatment or, can include a series of treatments.

As indicated herein, the oral or mucosal of soybean extracts of the invention can also include one or more therapeutic agents useful for treating an immune-related disorder. Such therapeutic agents can include, e.g., NSAIDs (including COX-2 inhibitors); other antibodies, e.g., anti-cytokine antibodies, gold-containing compounds; immunosuppressive drugs (such as corticosteroids, e.g., prednisolone and methyl prednisolone; cyclophosphamide; azathioprine; mycophenolate mofetil (MMF); cyclosporin and tacrolimus; methotrexate; or cotrimoxazole) and heat shock proteins.

The pharmaceutical compositions can be included in a container, pack, or dispenser together with instructions for administration.

One of ordinary skill in the art would readily appreciate that the pharmaceutical compositions described herein can be prepared by applying known pharmaceutical manufacturing procedures. Such formulations can be administered to the subject with methods well-known in the pharmaceutical arts. Thus, the practice of the present methods will employ, unless otherwise indicated, conventional techniques of pharmaceutical sciences including pharmaceutical dosage form design, drug development, and pharmacology, as well as of organic chemistry, including polymer chemistry. Accordingly, these techniques are within the capabilities of one of ordinary skill in the art and are explained fully in the literature.

More particularly, the immunomodulatory methods of treatment or prevention described by the invention typically include administering to a subject an oral or mucosal of soybean extracts, specifically, Femarelle (DT56a) sufficient to stimulate the mucosal immune system.

The usefulness of an oral formulation requires that the active agent or combinations of the invention be bio-available.

Bioavailability of orally administered drugs can be affected by a number of factors, such as drug absorption throughout the gastrointestinal tract, stability of the drug in the gastrointestinal tract, and the first pass effect. Thus, effective oral delivery of an active agent or combination requires that the active agent have sufficient stability in the stomach and intestinal lumen to pass through the intestinal wall. Many drugs, however, tend to degrade quickly in the intestinal tract or have poor absorption in the intestinal tract so that oral administration is not an effective method for administering the drug.

More specifically, the composition of the invention may be suitable for mucosal administration, for example, pulmonary, buccal, nasal, intranasal, sublingual, rectal, vaginal administration and any combination thereof.

Pharmaceutical compositions suitable for oral administration are typically solid dosage forms (e.g., tablets) or liquid preparations (e.g., solutions, suspensions, or elixirs).

Solid dosage forms are desirable for ease of determining and administering dosage of active ingredient, and ease of administration, particularly administration by the subject at home.

Liquid dosage forms also allow subjects to easily take the required dose of active ingredient. Liquid preparations can be prepared as a drink, or to be administered, for example, by a nasal-gastric tube (NG tube). Liquid oral pharmaceutical compositions generally require a suitable solvent or carrier system in which to dissolve or disperse the active agent, thus enabling the composition to be administered to a subject. A suitable solvent system is compatible with the active agent and non-toxic to the subject. Typically, liquid oral formulations use a water-based solvent.

The oral compositions of the invention can also optionally be formulated to reduce or avoid the degradation, decomposition, or deactivation of the active agents by the gastrointestinal system, e.g., by gastric fluid in the stomach. For example, the compositions can optionally be formulated to pass through the stomach unaltered and to dissolve in the intestines, i.e., enteric coated compositions.

According to one embodiment, the composition of the invention may be a pharmaceutical composition, nutraceutical composition, functional food, functional nutrition product, medical food, medical nutrition product or dietary supplement.

The terms “nutraceutical” combines the words “nutrition” and “pharmaceutical”. It is a food or food product that provides health and medical benefits, including the prevention and treatment of disease. A nutraceutical is a product isolated or purified from foods that is generally sold in medicinal forms not usually associated with food. A nutraceutical is demonstrated to have a physiological benefit or provide protection against chronic disease. Such products may range from isolated nutrients, dietary supplements and specific diets to genetically engineered foods, herbal products, and processed foods such as cereals, soups, and beverages. Nutraceutical foods are not subject to the same testing and regulations as pharmaceutical drugs.

The term “nutraceutical” as used herein denotes usefulness in both nutritional and pharmaceutical fields of application. Thus, novel nutraceutical compositions can be used as supplements to food and beverages and as pharmaceutical formulations for enteral or parenteral application which may be solid formulations, such as capsules or tablets, or liquid formulations, such as solutions or suspensions.

The nutraceutical compositions according to the present invention may further contain protective hydrocolloids (such as gums, proteins, modified starches), binders, film-forming agents, encapsulating agents/materials, wall/shell materials, matrix compounds, coatings, emulsifiers, surface active agents, solubilising agents (oils, fats, waxes, lecithins etc.), adsorbents, carriers, fillers, co-compounds, dispersing agents, wetting agents, processing aids (solvents), flowing agents, taste-masking agents, weighting agents, jellyfying agents, gel-forming agents, antioxidants and antimicrobials.

Moreover, a multi-vitamin and mineral supplement may be added to nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

If the nutraceutical composition is a pharmaceutical formulation the composition further contains pharmaceutically acceptable excipients, diluents or adjuvants. Standard techniques may be used for their formulation, as e.g. disclosed in Remington's Pharmaceutical Sciences, 20th edition Williams & Wilkins, PA, USA. For oral administration, tablets and capsules are preferably used which contain a suitable binding agent, e.g. gelatine or polyvinyl pyrrolidone, a suitable filler, e.g. lactose or starch, a suitable lubricant, e.g. magnesium stearate, and optionally further additives.

The soybean extracts of the invention and any combinations thereof can be administered from one or more times per day to one or more times per week, including once every other day. The oral or mucosal soybean extracts compositions can be administered, e.g., for about 1 to 30, 5 to 14 days or longer. The skilled artisan will appreciate that certain factors may influence the dosage and timing required to effectively treat a subject, including but not limited to the severity of the disease or disorder, previous treatments, the general health and/or age of the subject, and other diseases present.

Moreover, treatment of a subject with a therapeutically effective amount of the soybean extracts can include a single treatment or, can include a series of treatments.

More particularly, since the present invention relates to the treatment of diseases and conditions with a combination of active ingredients which may be administered separately, the invention also relates as a further aspect, to combining separate pharmaceutical compositions in kit form. The kit includes at least two separate pharmaceutical compositions selected from: (i) at least one soybean extract, optionally in a pharmaceutical dosage form; (ii) at least one enzymatic soybean extract, optionally in a pharmaceutical dosage form; (iii) at least one hexane soybean extract, optionally in a pharmaceutical dosage form; (iv) at least one aqueous soybean extract, optionally in a pharmaceutical dosage form; and (v) at least one additional therapeutic agent, optionally in a pharmaceutical dosage form.

According to certain embodiments, the kit may be suitable for preventing acute or chronic toxic effect of an analgesic or an antipyretic drug or any type of liver insult. The insult is selected from infectious metabolic, toxic, immune, or perfusion or blood flow related hepatic injury. The kit according to this embodiment comprises: (a) at least one soybean extract, optionally in a first pharmaceutical dosage form; (b) at least one additional therapeutic agent, wherein the agent is an analgesic or an antipyretic drug, optionally in a second pharmaceutical dosage form; and (c) optionally, container means for containing the first and second dosage forms.

In another specific embodiment, said analgesic or antipyretic drug may be an inducer or inhibitor of Cytochrom P-450 selected from the group consisting of: Acetaminophen, Phenobarbital, Phenytoin, Carbamazepine, Primidone, Ethanol, Glucocorticoids, Rifampin, Griseofulvin, Quinine, Omeprazole, Amiodarone, Cimetidine, Erythromycin, Grape fruit, Isoniazid, Ketoconazole, Metronidazole, Sulfonamides, Chlorpromazine, phenylbutazone, halogenated anesthetic agents, sulindac, Dapsone, INH, halothane, amoxicillin-clavulanic acid, phenobarbital, Para-amino salicylate, Clofibrate, Procainamide, Gold salts, propylthiouracil, chloramphenicol, nitrofurantoin, methoxyflurane, penicillamine, paraquat, Tetracycline, Contraceptive and anabolic steroids, rifampin, Aspirin and Sodium valproate.

In a more specific embodiment, said analgesic drug is acetaminophen (paracetamol).

Thus, according to one embodiment, the enzymatic soybean extract comprised in the kit of the invention is Femarelle (DT56a) or any ethanol extract thereof, and the analgesic or antipyretic drug is acetaminophen (paracetamol).

According to other embodiments, the kit of the invention is for treating, ameliorating preventing or delaying the onset of any one of hepatic disorder, drug induced hepatic injury, the Metabolic Syndrome or an immune-related disorder in a subject in need thereof. In such embodiments, the kit comprises: (a) at least one aqueous soybean extract M1, optionally in a first pharmaceutical dosage form; (b) at least one hexane soybean extract OS, optionally in a second pharmaceutical dosage form; and (c) optionally, container means for containing the first and second dosage forms.

More specifically, the kit includes container means for containing at least both separate compositions; such as a divided bottle or a divided foil packet however, the separate compositions may also be contained within a single, undivided container. Typically the kit includes directions for the administration of the separate components. The kit form is particularly advantageous when the separate components are preferably administered in different dosage forms (e.g., oral and nasal, dermal or parenteral), are administered at different dosage intervals, or when titration of the individual components of the combination is desired by the prescribing physician.

Achieving a therapeutic effect is meant for example, where the kit is intended for the treatment of a specific disorder, the therapeutic effect may be for example slowing the progression of the treated condition.

The invention further provides a method of treating, ameliorating, preventing or delaying the onset of an immune-related disorder in a subject in need thereof comprising the step of administering to said subject a therapeutically effective amount of a first and a second unit dosage forms comprised in a kit according to the invention.

It should be appreciated that the components of the kit, the different soybean extracts of the invention and optionally, the additional therapeutic agent, may be administered simultaneously.

Alternatively, said first compound or dosage form and said second compound or dosage form are administered sequentially in either order.

More specifically, the kits described herein can include combined soybean extracts or an oral compositions thereof in separate dosage unit forms, as an already prepared liquid oral dosage form ready for administration or, alternatively, can include the combined soybean extracts of the invention as a solid pharmaceutical composition that can be reconstituted with a solvent to provide a liquid oral dosage form. When the kit includes the combined soybean extracts of the invention as a solid pharmaceutical composition that can be reconstituted with a solvent to provide a liquid dosage form (e.g., for oral or nasal administration), the kit may optionally include a reconstituting solvent. In this case, the constituting or reconstituting solvent is combined with the active ingredient to provide liquid oral dosage forms of each of the active ingredients or of a combination thereof. Typically, the active ingredients are soluble in so the solvent and forms a solution. The solvent can be, e.g., water, a non-aqueous liquid, or a combination of a non-aqueous component and an aqueous component. Suitable non-aqueous components include, but are not limited to oils, alcohols, such as ethanol, glycerin, and glycols, such as polyethylene glycol and propylene glycol. In some embodiments, the solvent is phosphate buffered saline (PBS).

In yet another aspect, the invention is directed to a method for increasing the maximum amount of acetaminophen administered to a subject without exhibiting acetaminophen toxicity. This method comprises administering of an acetaminophen toxicity inhibiting amount of a soybean extract, any enzymatically processed product or derivatives thereof, or any composition or mixture comprising the same, before, simultaneously with, after or any combination thereof, administration of the acetaminophen to the subject.

It should be appreciated that such simultaneous administration may be performed by administering the soybean extracts of the invention, specifically, Femarelle or M1, OS and combinations thereof, and acetaminophen.

According to one embodiment, the soybean extracts of the invention, specifically, any enzymatic, hexane, ethanol or aqueous soybean extract, more specifically, Femarelle (DT56a) or M1, OS, M0-1, M0-2, and T1, extracts used by the invention may be administered within forty-eight to ninety-six hours of the administration of acetaminophen to said subject, or at any time point before or after administration of the toxic drug, or at any time point before or after any type of liver insults due to infectious, metabolic, toxic, immune, perfusion or blood flow reasons occurred.

The soybean extract the soybean extract of the invention, specifically, any enzymatic, hexane, ethanol or aqueous soybean extract, more specifically, such soybean extract may be Femarelle (DT56a) or M1, OS, M0-1, M0-2, and T1, compositions can be administered from one or more times per day to one or more times per week, including once every other day. It can be administered, e.g., for about 1 to 30, 5 to 14 days or longer. The skilled artisan will appreciate that certain factors may influence the dosage and timing required to effectively treat a subject, including but not limited to the severity of the disease or disorder, previous treatments, the general health and/or age of the subject, and other diseases present.

Moreover, treatment of a subject with a therapeutically effective amount of the combined compounds can include a single treatment or, can include a series of treatments.

It should be appreciated that the present invention provides a method of inducing at least one of T regulatory cells in a subject in need thereof, specifically, a subject suffering from acute or chronic effect of acetaminophen. The method of the invention comprises the step of administering to said subject a therapeutically effective amount of at least one of:

(a) soybean extract of the invention, specifically, any enzymatic, hexane, ethanol or aqueous soybean extract, more specifically, Femarelle (DT56a) or M1, OS, M0-1, M0-2, and T1;
(b) an immune-cell treated with (a) or with any composition comprising the same;
(c) an immune-cell obtained from a subject treated with any one of (a), (b) or any combinations or mixtures thereof or any composition comprising the same; and
(d) a composition comprising ay one of (a), (b), (c) or any combinations or mixtures thereof, said composition optionally further comprises at least one pharmaceutically acceptable carrier, diluent, excipient and/or additive.

By “patient” or “subject in need” it is meant any mammal who may be affected by the above-mentioned conditions, and to whom the treatment and diagnosis methods herein described is desired, including human, bovine, equine, canine, murine and feline subjects. Preferably said patient is a human. Administering of the drug combination to the patient includes both self-administration and administration to the patient by another person.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician.

Unless otherwise defined, all technical and scientific terms used herein have the same meaning as commonly understood by one of ordinary skill in the art to which this invention belongs. Although methods and materials similar or equivalent to those described herein cannot be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventor in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Materials

DT56a (Tofupill/Femarelle, Se-cure Pharmaceuticals, Dalton, Israel)

Cremophor EL (Sigma, Rehovot, Israel)

Concanavalin A (ConA; MP Biomedicals, Ohio, USA)

High fat diet (TD88137, Harlan)

CD4/CD3-Pacific Blue, CD25/NK1.1-PE, Foxp3-PE-Cy7 and CD8-FITC antibodies (eBioscience, San Diego, Calif., USA)

Fixation buffer (eBioscience, San Diego, Calif., USA)

Foxp3 staining buffer (eBioscience, San Diego, Calif., USA)

Equipment

Reflovet Plus clinical chemistry analyzer (Roche Diagnostics, GmbH, Mannheim, Germany)

TUNEL assay kit (Roche diagnostics, Germany).

Axioplan fluorescent microscope (Zeiss, Oberkochen, Germany)

Charge-coupled device camera (Quantix Corp., USA)

GPO-Trinder kit (Sigma, Rehovot, Israel)

IFN-γ, TNF-α, and IL-6 ELISA (Quantikine, R&D Systems, Minneapolis, Minn., USA)

Lymphoprep (Ficoll, Axis-Shield PoC AS, Oslo, Norway)

FACS LSR II (Becton Dickinson, San Jose, Calif.)

FCS express V.3 software (DeNovo software, CA, USA)

### EXPERIMENTAL PROCEDURES

Femarelle

Dosages of DT56a were administered orally to all animals. DT56a was emulsified in a mixture of 15% Cremophor EL and 15% ethanol (C:E) in water. Dosages of oral DT56a tested in all models were 1 μg (low dose) and either 53 μg or 56 μg (high dose), as indicted. A mixture of 15% Cremophor EL and 15% ethanol (C:E) in water (vehicle) was orally administered to control groups.

Preparation of secondary Femarelle extracts is described in Example 4.

Soybean Extracts

M1, M0-1, M0-2, OS and T1 are extracts produced by Solbar. OS comprises all soybean lipids that are dissolved in hexane. The OS vehicle is Cremophor-CEEthanol (C:E) in a 1:1 ratio (v/v) in 90% PBS. Cremophor-EL is an emulsifying agent for the pharmaceuticals, cosmetics and foodstuffs industries; used in aqueous preparations of hydrophobic substances, e.g. fat-soluble vitamins and essential oils. It is also known as Polyoxyethylenglyceroltriricinoleat 35 (DAC) and Polyoxyl 35 Castor Oil (USP/NF). M1 comprises 50% of dry matter, 60% of which is sucrose and the rest is raffinose and stachyose. 8% are proteins. There are also isoflavins (1%), saponins (2%), minerals, lipids and other components. M-01, M-02 and T1 are derived from M1, and have unknown compositions. M1, M-01 and M-02 vehicle is DDW, and the T1 vehicle is PBS. GC is a natural β-glycolipid. GC was prepared as an emulsion in Cremophor-Cl:Ethanol (C:E) in a 1:1 ratio (v/v) in 70% PBS. Femarelle, also known as DT56a and Tofupill, and sometimes referred to herein as F-1, is a natural compound that is an enzymatic isolate of soybeans. All extracts were stored in −20° C.

Concanavalin A

Concanavalin A (ConA) (0.5 mg, 20 mg/kg) was dissolved in 200 μl of 50 mM Tris pH 7, 150 mM NaCl, 4 mM CaCl₂, and injected intravenously into mice.

Acetaminophen

Acetaminophen was dissolved in a mixture of 30% Cremophor EL and ethanol (1:1) in DDW.

Animals

Adult (aged 11-12 weeks) male wild-type C57BL-6 (B6) mice and Female C57Bl/6 mice (16 weeks old) were purchased from Harlan Laboratories (Jerusalem, Israel) and were used for the ConA and the acetaminophen challenges. Male B6 mice (aged 6-8 weeks) were used for the high-fat diet (HFD) model. In this diet, 42% of calories were from fat (Harlan, TD88137), and the mice were fed this diet for 12 weeks. Male (aged 6-8 weeks) leptin-deficient mice on a C57Bl/6 background were used for the NASH model. These mice were also purchased from Harlan Laboratories [Beyan H. et al., Diabetes/metabolism research and reviews. 19(2):89-100 (2003)]. All mice were maintained in the Animal Core of the Hadassah-Hebrew University Medical School. The mice were given standard laboratory chow (except for the HFD mice) and water ad libitum and kept in a 12-hour light/dark cycle. All animal experiments were carried out in accordance with the guidelines of the Hebrew University-Hadassah Institutional Committee for Care and Use of Laboratory Animals and with the committee's approval.

ConA Challenge

Either two (Example 1 (A)) or three (Example 1 (B)) groups of mice (10 mice per group) were studied. ConA was dissolved in 50 mM Tris (pH 7), 150 mM NaCl, and 4 mM CaCl₂. To induce autoimmune hepatitis, mice were injected i.v. with 20 mg/kg ConA. Control mice were injected with PBS. A specified dosage of Femarelle or vehicle was administered to the mice orally 30 mM before (Example 1 (A)) or 30 min after (Example 1 (B)) they had received an injection of ConA. The mice were sacrificed 16 h (Example 1 (A)) or 17 h (Example 1 (B)) later. For Example 4 (ConA challenge in different Femarelle extracts), six groups of 4-5 11-12 weeks old male C57BL/6 mice were treated per os for three days with extracts as show in Table 4. The mice were injected i.v. (tail vein) 500 μg of ConA, (20 mg/kg body weight) and sacrificed 14 h later. After sacrifice measurements of serum IFN-γ was carried out using ELISA. For Example 6 (ConA challenge in different soybean extracts), 11-12 weeks old male C57BL/6 mice (4-6 per group) were administered the indicated amount of soybean extract selected from OS, M1, M-01, M-02, T1 as well as F-1 and GC per os each day for three days prior to ConA injection. In all experiments 5 mg (20 mg/kg body weight) ConA were injected i.v. (tail vein) to all mice. Mice were sacrificed 14 hours after administration of ConA, and blood was cardially withdrawn and separated to serum and plasma. Serum ALT and AST activities were determined and serum levels of IFN-γ and TNF-α were measured.

Acetaminophen Challenge

For the acetaminophen intoxication model, either two (Example 1 (A)) or three (Example 1 (B)) groups of mice (6 mice per group) were studied Animals were fasted overnight (8 h) before treatment with acetaminophen (400 mg/kg in C:E in water) or vehicle via a stomach tube. Femarelle doses of 1 μg (low dose), 53 μg or 56 μg (high dose), or vehicle, as indicted, were orally administered to mice 120 minutes before (Example 2 (A)) or 60 min after (Example 2 (B)) acetaminophen administration Animals were sacrificed 20 h (Example 2 (A)) or 24 h (Example 2 (B)) following acetaminophen administration.

HFD Challenge

Five mice per experimental group were fed HFD diet, comprising 42% calories from fat. The mice were administered the indicated extracts per os 3 times a week for three months, and then sacrificed. The mice weight, fasting glucose levels, ALT, serum triglycerides (TGs) and cholesterol were determined every 2 weeks. Glucose Tolerance Test was performed after 4 and 12 weeks. On sacrifice day, serum and a liver biopsy were collected for determination of serum insulin levels, liver TG and for H&E histological analysis.

After sacrifice, measurements of serum ALT and AST activities were carried out, and serum levels of IFN-γ, TNF-α and insulin were determined.

Animal Models of NAFLD

Three groups of leptin-deficient (ob/ob) mice (5 mice per group) were given vehicle or Femarelle daily for six weeks. Three groups of mice receiving HFD (6 mice per group) were given vehicle or Femarelle three times a week for eleven weeks.

ob/ob mice were monitored weekly for weight and levels of fasting blood glucose, ALT, and AST. A glucose tolerance test (GTT) was performed during the 5th week. HFD mice were monitored every two weeks for fasting glucose levels, serum triglycerides (TGs) and total cholesterol. Liver enzymes were monitored every 4 weeks. A GTT was performed during weeks 4 and 8.

Alanine Aminotransferase and Aspartate Aminotransferase Assays

Measurements of serum alanine aminotransferase (ALT) and aspartate aminotransferase (AST) activities were carried out using a Reflovet Plus clinical chemistry analyzer.

Histology

For histopathology, livers from individual mice were fixed in 10% formaldehyde solution and kept at room temperature until use. The tissue blocks were then embedded in paraffin, sectioned and stained with hematoxylin and eosin (H&E) for morphological examination. Specimens were examined under a light microscope.

Apoptosis Assay

Hepatocellular apoptosis in the ConA model was determined by an in situ terminal deoxynucleotidyl transferase-mediated dUTP nick end-labeling (TUNEL) assay using a commercial kit. Briefly, paraffin-embedded liver tissues were cut, deparaffinized and hydrated according to standard procedures. Fluorescent cells in hepatic tissues were digitally photographed using an Axioplan fluorescent microscope under a high-power magnification (×200). Images were collected with a cooled charge-coupled device camera using Image Pro software.

Lipid Accumulation in the Liver

TGs were extracted from aliquots of snap-frozen livers using a modification of the Folch method. Hepatic TG content was assayed spectrophotometrically using a GPO-Trinder kit and was normalized to the protein content in the homogenate.

Measurement of Plasma Lipids

Plasma TG and total cholesterol were measured by a clinical chemistry analyzer, the Reflovet Plus machine.

Fasting Glucose Levels

Fasting glucose levels were measured after overnight (12 h) fasting. Serum glucose measurements were performed on tail-vein blood by a standard glucometer.

Glucose Tolerance Test

Mice underwent a GTT after overnight fasting. Glucose was administered orally (1.25 g/kg body weight). Serum glucose measurements were performed on tail-vein blood every 15 min for 3 h. Glucose levels were measured by a standard glucometer.

Insulin Determination

Serum insulin levels were determined using a commercially available ELISA kit (Mercodia), according to the manufacturer's instructions. Serum was collected from euthanized mice on the day of sacrifice (after 12 h fasting) and kept in −80° C. until analysis.

Cytokine Determination

Serum levels of IFN-γ, TNF-α, and IL-6 were determined by “sandwich” ELISA using commercial kits according to the manufacturer's instructions.

Isolation of Splenocytes and Intrahepatic Lymphocytes

Livers and spleens were stored in RPMI-1640 supplemented with FCS. Spleens were crushed through a 70-μm nylon cell strainer [Falcone M. et al., J. Immunol. 172(10):5908-5916 (2004)] and centrifuged (1250 rpm for 7 min) Red blood cells were lysed in 1 ml of cold 155 mM ammonium chloride lysis buffer. Splenocytes were washed and resuspended in 1 ml of RPMI supplemented with FCS. The viability of cells as assessed by trypan blue exclusion exceeded 90%. For intrahepatic lymphocytes, livers were crushed through a stainless mesh (size 60, Sigma). Ten milliliters of Lymphoprep was loaded with a similar volume of the cell suspension in 50-ml tubes. The tubes were centrifuged at 1800 rpm for 18 min Cells present in the interface were collected and centrifuged again at 1800 rpm for 10 min to obtain a pellet of cells depleted of hepatocytes. Approximately 1×106 cells/mouse liver were recovered.

Flow Cytometry for Lymphocyte Subsets

Flow cytometry was performed following splenocyte and hepatic lymphocyte isolation using 1×10⁶ lymphocytes in 100 μl PBS with 0.1% BSA. For surface staining, cells were incubated with fluorochrome-conjugated antibodies to the indicated cell surface markers at the recommended dilutions or with isotype control antibodies for 30 minutes at 4° C. The following cell surface anti-mouse antibodies were used: CD4/CD3-Pacific Blue, CD25/NK1.1-PE and CD8-FITC. Cells were then washed in PBS containing 1% BSA and fixed with fixation buffer for another 50 minutes. For intracellular staining of Foxp3, fixed cells were permeabilized with Foxp3 staining buffer. Cells were then stained with PE-Cy7-conjugated antibodies to Foxp3, washed twice and resuspended in 250 μl of PBS containing 1% BSA and kept at 4° C. One million stained cells in 250 μl of PBS containing 1% BSA were subsequently analyzed using a FACS LSR II instrument with FCS express V.3 software. Only live cells were counted, and background fluorescence from non-antibody-treated lymphocytes was subtracted.

Statistical Analysis

The comparison of two independent groups was performed using the Student's t-test. All applied tests were two-tailed, and a p value of 0.05 or less was considered statistically significant.

### Example 1

Femarelle in the Treatment of Immune-Related Disorders

A. Pre-Treatment with Femarelle Prevents Immune Mediated Damage in ConA Hepatitis Model

The Concanvalin A (ConA) model is a widely utilized mouse model which mimics many aspects of human autoimmune hepatitis. Concanavalin A is a bean lectin, which when injected intravenously to mice, induces activation of Natural Killer T (NKT) cells in the liver. Together with Kupffer cells, NKT cells secrete large amounts of various hepatotoxic cytokines, most notably IFN-γ/and TNF-α. ConA induces massive liver necrosis in mice with high level of apoptotic hepatocytes and elevated serum liver enzymes (ALT and AST), hallmarks of acute inflammation. Thus, the inhibition of ConA-induced ALT and AST increase, as well as other induced markers of liver damage, indicates an effective treatment.

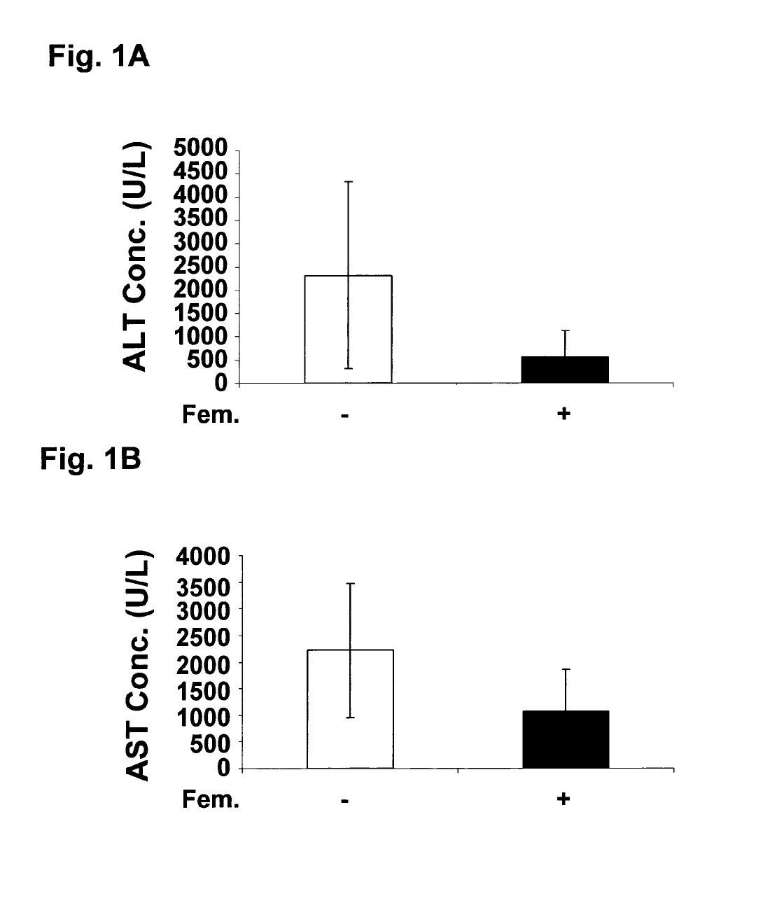

In order to ascertain the beneficial effects of Femarelle in prevention of hepatic inflammatory damage, female C57Bl/6 mice were administered Femarelle orally 0.5 hours prior to an intravenous ConA injection. Alanine transaminase (ALT) and Aspartate aminotransferase (AST) were determined 16 hours following ConA administration. As shown in FIG. 1, Femarelle ameliorated ConA-induced liver damage as demonstrated by lower ALT (1A) and AST (1B) levels in Femarelle pre-treated mice.

B. Oral Administration of Femarelle Alleviated Immune-Mediated Hepatitis

To evaluate the potential of Femarelle for treatment of immune-related hepatic disorders, rather than prophylaxis thereof, three groups of mice (10 mice per group) were administered either 1 μg (low dose) or 53 μg (high dose) Femarelle or vehicle 30 min after they had received an injection of ConA. The mice were sacrificed 17 h later.

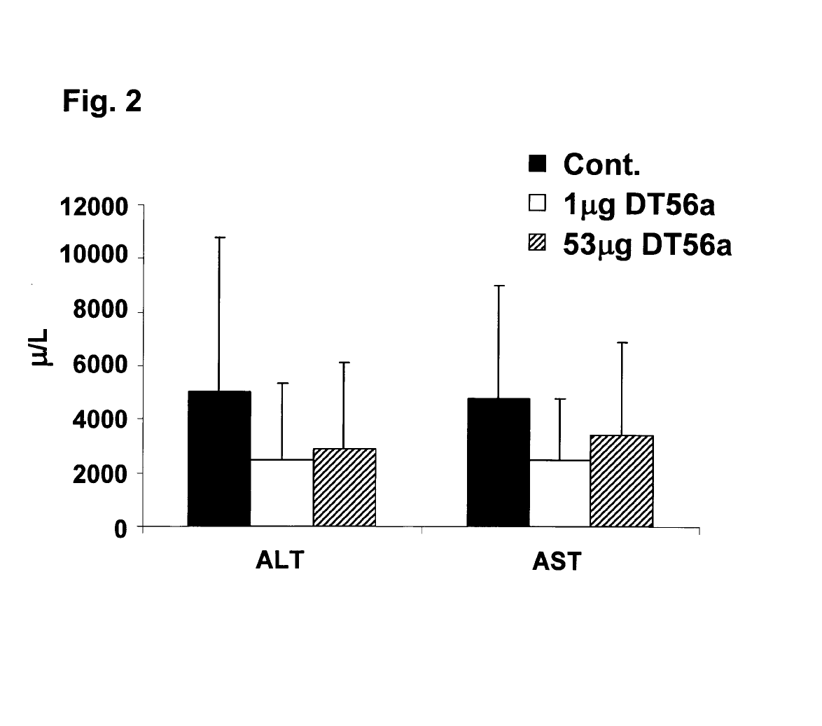

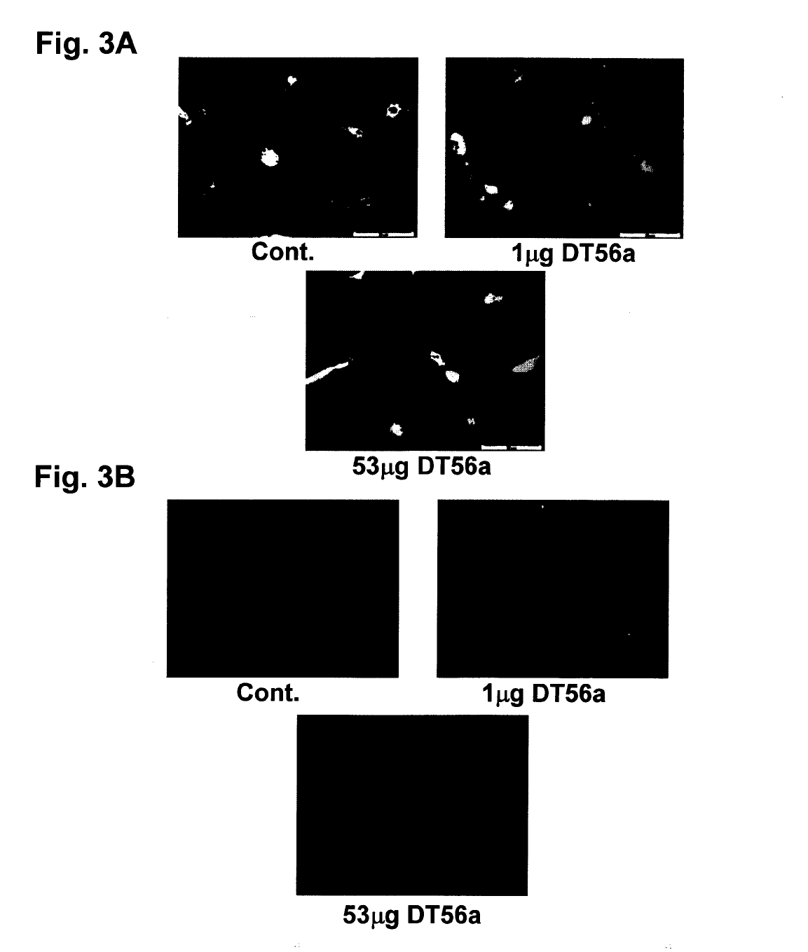

FIG. 2 shows the effect of oral administration of Femarelle on immune-mediated liver damage induced by ConA. The data shows that oral administration of Femarelle decreased levels of ALT and AST liver enzymes (p=NS), and FIG. 3A shows representative H&E-stained liver sections. Histological examination demonstrated diffuse and massive infiltration and severe necrosis in control mice. These symptoms were reduced in Femarelle-treated animals. FIG. 3B shows a decrease in hepatic apoptosis in treated animals as depicted by TUNEL staining.

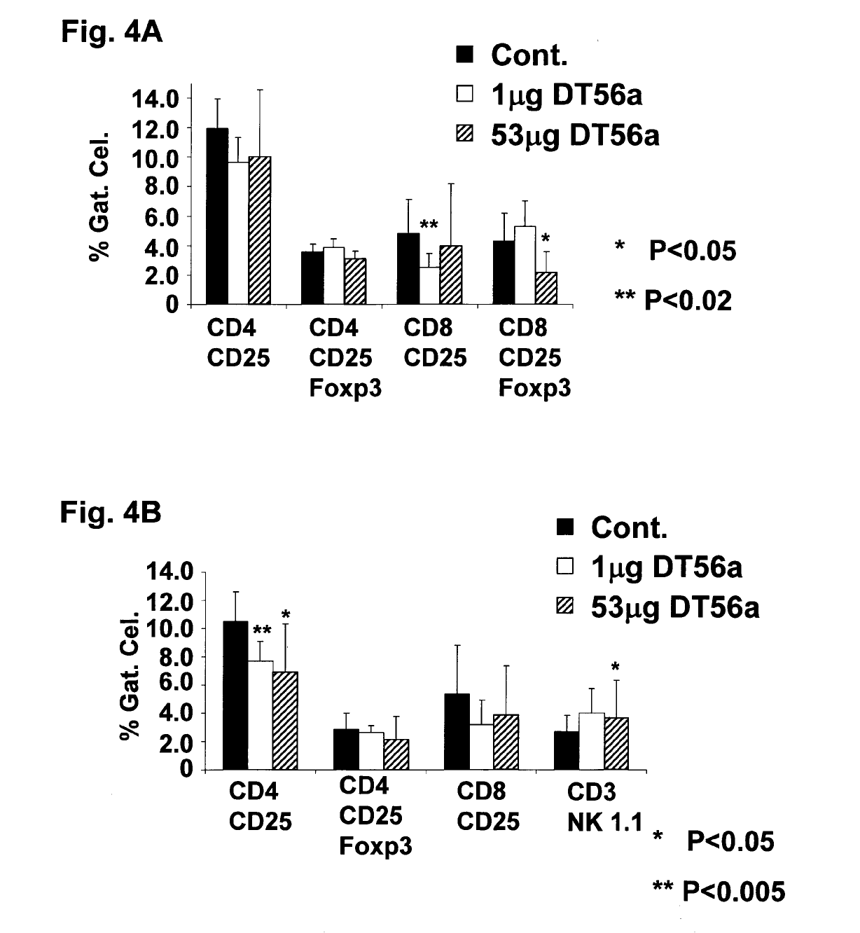

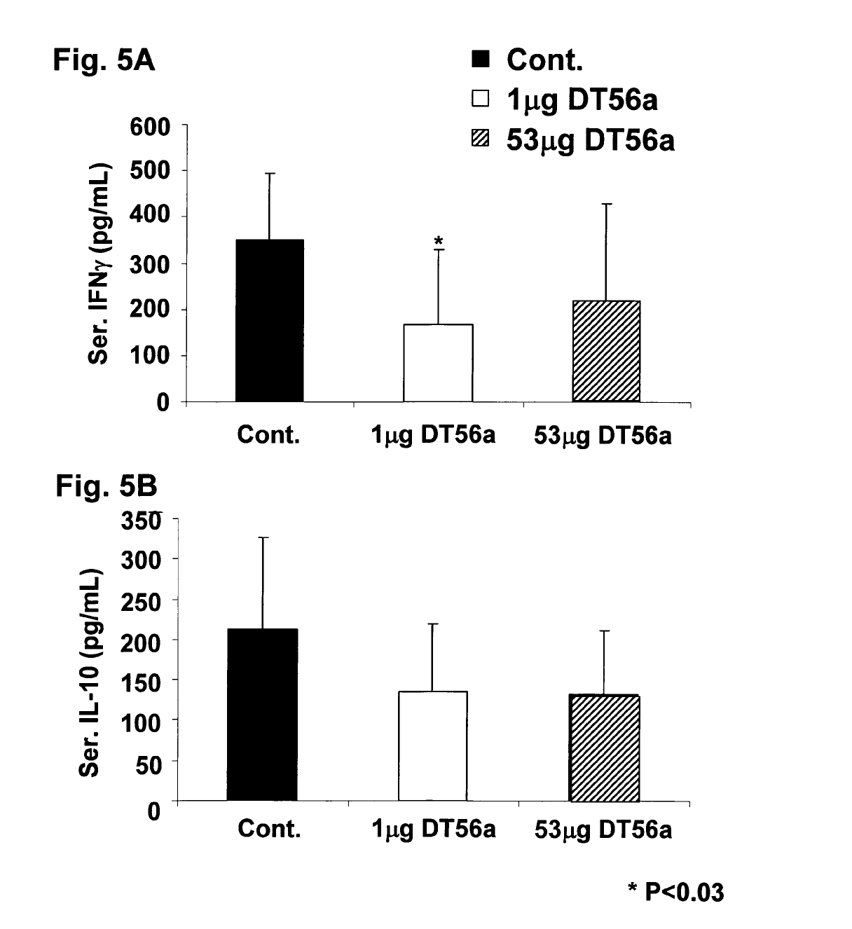

The hepatoprotective effect of Femarelle was associated with a redistribution of Tregs in the liver and spleen. FIG. 4A shows that oral administration of 1 μg of Femarelle significantly decreased CD4⁺CD25⁺ and CD8⁺CD25⁺ cells in the spleen (p<0.02). Administration of a high dose of Femarelle (53 fig) significantly decreased CD8⁺CD25⁺FOXP3⁺ cells (p<0.05). FIG. 4B shows that in the liver, CD4⁺CD25⁺ cells were significantly decreased by both dosages of Femarelle (p<0.005 for the low dose; p<0.05 for the 53 μg dose). CD3⁺NK1.1⁺ cells were significantly increased after oral administration of a low dose of Femarelle (p<0.05). Cytokines play an important role in mediating the liver damage induced by ConA. FIG. 5A shows that serum IFN-γ levels were significantly decreased by 52% after administration of a low dose of Femarelle (p<0.03), and FIG. 5B shows a decrease in serum IL-10 in mice treated with either high (53 fig) or low (1 mg) dose Femarelle. Taken together, the data suggest that Femarelle exerts a hepatoprotective effect in mice with immune-mediated liver damage.

### Example 2

Femarelle in the Treatment and Prevention of Drug Induced Liver Damage

A. Femarelle Prevents Acetaminophen-Mediated Liver Damage

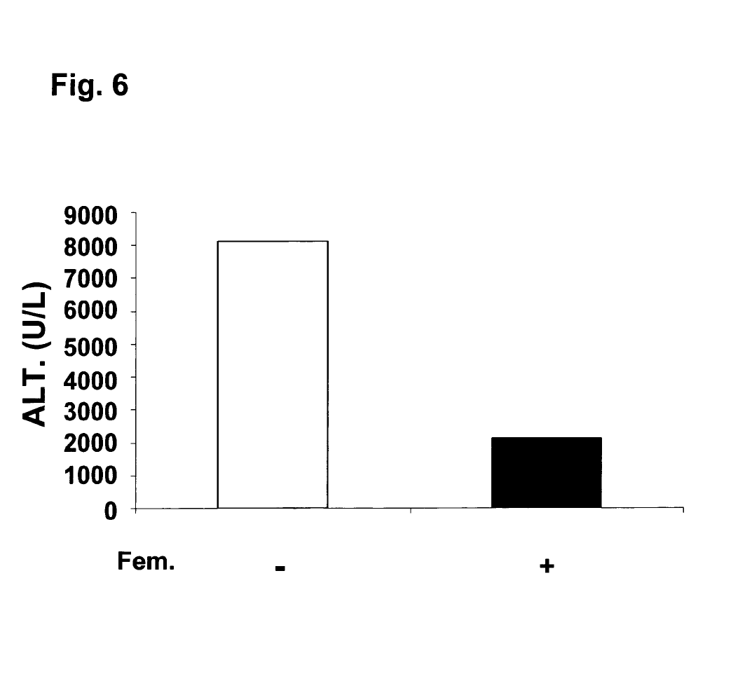

The inventor next evaluated the efficacy of Femarelle in prevention of acetaminophen-mediated liver damage. Female C57Bl/6 mice were administered. Femarelle was administered orally 2 hours prior to an intravenous acetaminophen injection. Alanine transaminase (ALT) was determined 20 hours following acetaminophen administration. As can be seen in FIG. 6, Femarelle ameliorated acetaminophen-induced liver damage as demonstrated by lower ALT levels in Femarelle pre-treated mice.

B. Oral Administration of Femarelle Alleviated Drug-Induced Hepatitis

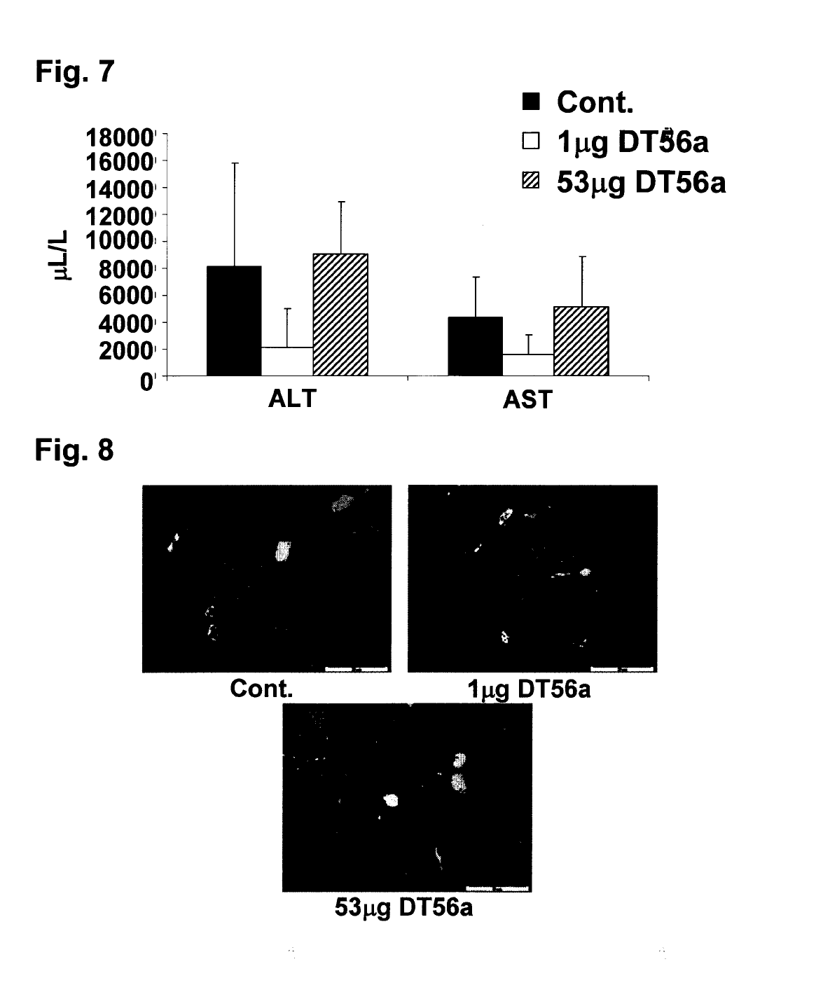

Showing the preventive effect of Femarelle, the inventor next evaluated the potential of Femarelle for treatment of drug-induced hepatic injuries, rather than prophylaxis thereof. FIG. 7 shows the effect of either 1 μg (low dose) or 53 μg (high dose) Femarelle on pre-existing acetaminophen-induced liver injury, i.e., administration of Femarelle 60 minutes after challenge with acetaminophen. The data shows that the elevation of ALT and AST serum levels in response to acetaminophen was reduced in mice treated with a low dose of Femarelle compared with control mice. FIG. 8 shows the hepatoprotective effect of Femarelle on liver histology. Representative liver sections of H&E staining demonstrate a decrease in the degree of injury in mice treated with a low dose of Femarelle. The lack of an effect in the mice treated with a high dose of Femarelle indicates a dose-dependent effect for this compound.

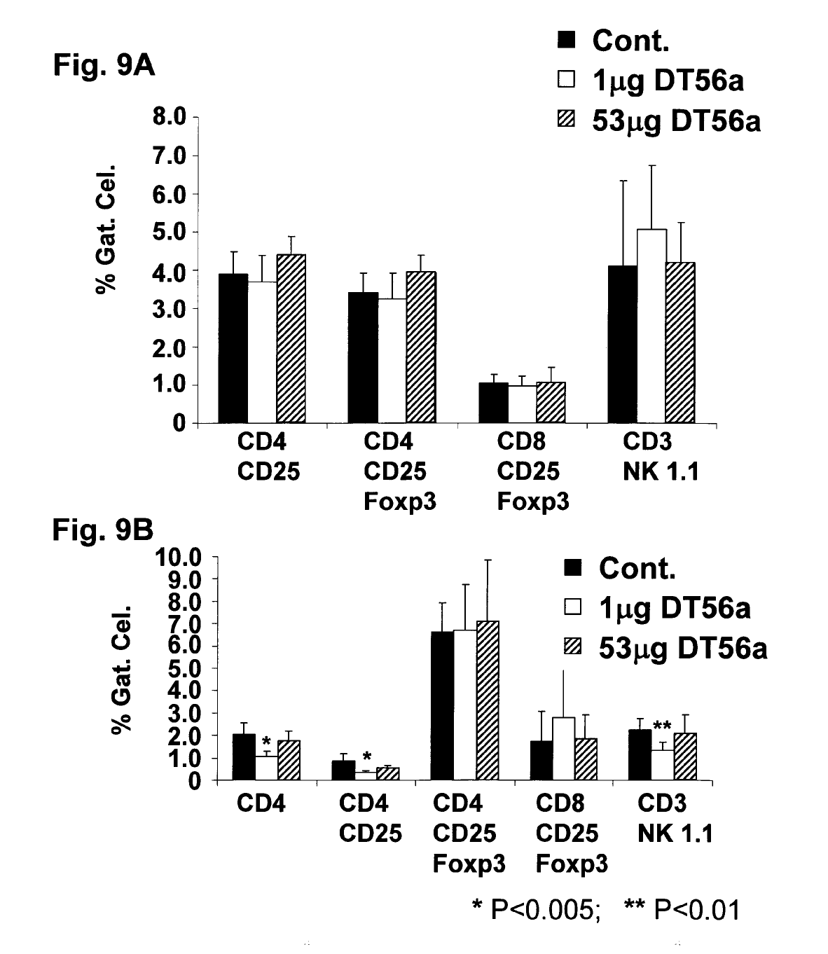

The immune system plays a role in mediating the liver damage that results from acetaminophen intoxication. FIG. 9A shows that a low dose of Femarelle induced an increase in CD3⁺NK1.1⁺ cells in the spleen. FIG. 9B shows that oral administration of 1 μg of Femarelle caused a significant decrease in CD25⁺ and CD4⁺CD25⁺ cells in the livers of acetaminophen-challenged mice (p<0.005). No significant changes were noted in the serum levels of TNF-α or IL10 (data not shown).

### Example 3

Femarelle in the Treatment of Metabolic Syndrome

A. Oral Administration of Femarelle Alleviated Metabolic Syndrome in ob/ob Mice

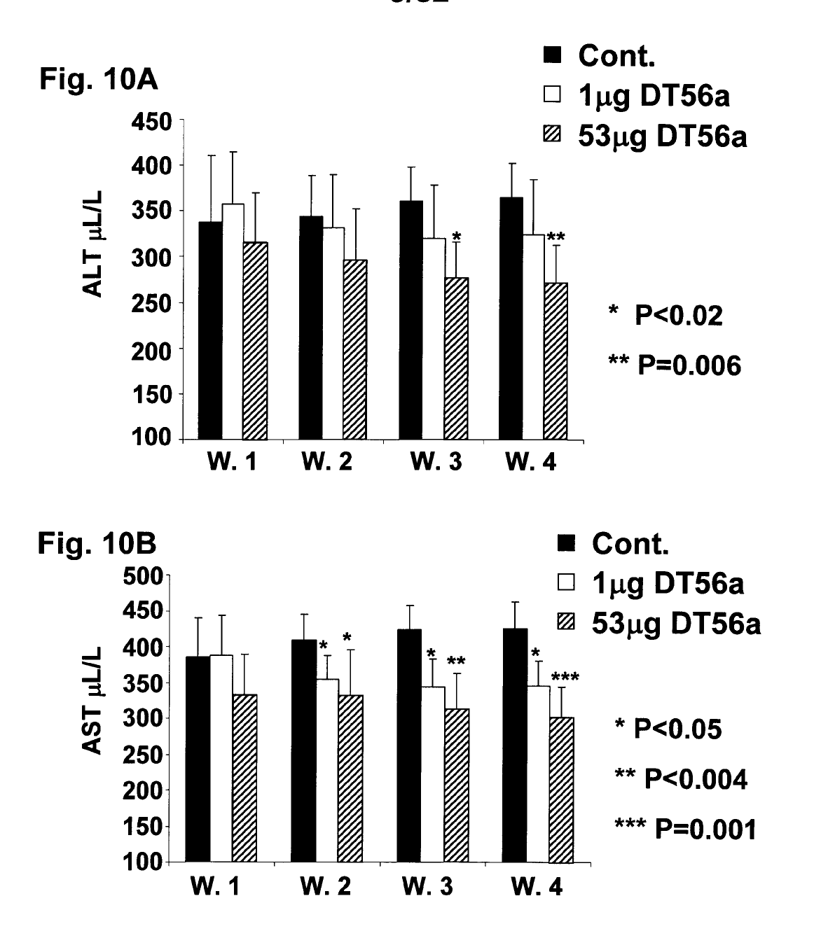

Two animal models were used for assessment of the effect of Femarelle on the liver damage associated with insulin resistance and metabolic syndrome. FIGS. 10-13 show the effect of oral administration of either 1 μg (low dose) or 53 μg (high dose) Femarelle for 6 weeks on the immune and metabolic parameters of ob/ob mice. FIGS. 10A and 10B show the effects on ALT and AST serum levels, respectively. Treatment with a high dose of Femarelle led to a significant decrease in ALT levels in weeks 3 and 4 (p<0.02 and p=0.006, respectively). AST levels were also significantly decreased by both dosages of Femarelle in week 2 (FIG. 10B).

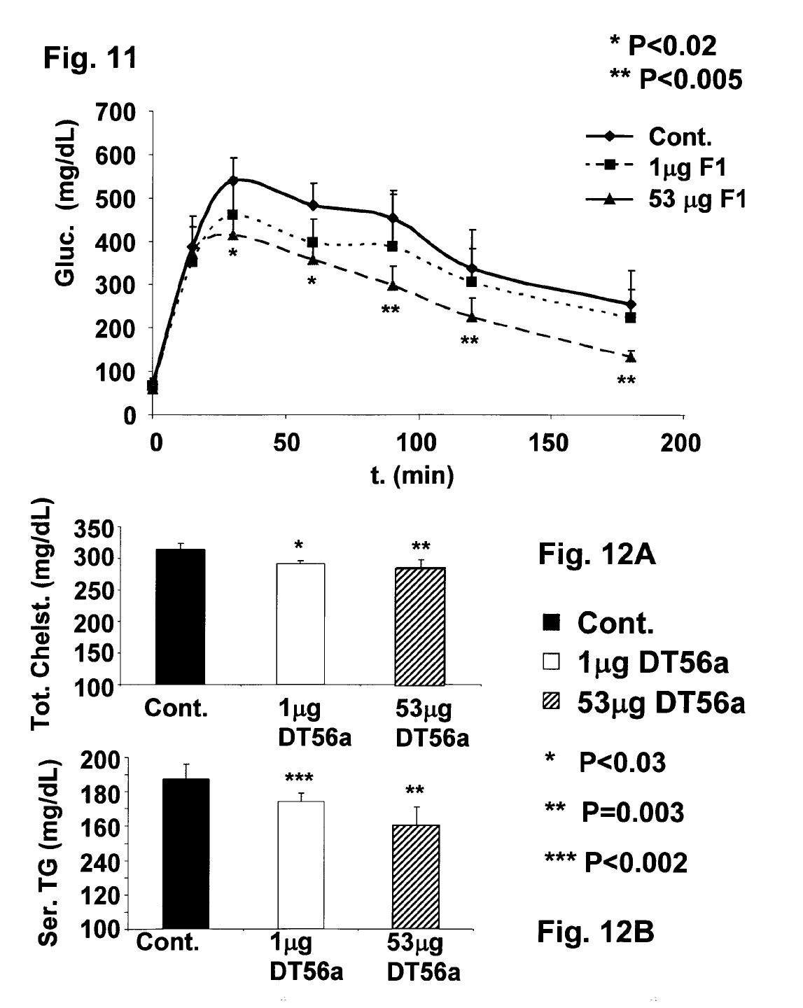

Oral administration of Femarelle improved insulin resistance, as indicated by the reductions in the elevated fasting blood glucose levels in weeks 3-6 (data not shown). FIG. 11 shows the results of GTTs performed in week 4 of treatment. Significant reductions in blood glucose levels were observed in mice treated with high dose of Femarelle (for time points 30 and 60 min, p<0.02; for time points 90, 120 and 180 min, P<0.005). FIG. 12 shows that the improved insulin resistance was associated with reductions in serum cholesterol (FIG. 12A) and triglycerides (FIG. 12B) levels. Serum cholesterol levels were significantly reduced by treatment with both dosages of Femarelle (p<0.003). Serum triglycerides levels were significantly decreased after 4 weeks of treatment with high (P=0.003) and low (P<0.03) dosages of Femarelle. The beneficial effects of Femarelle in obese mice were independent of changes in body weight (data not shown).

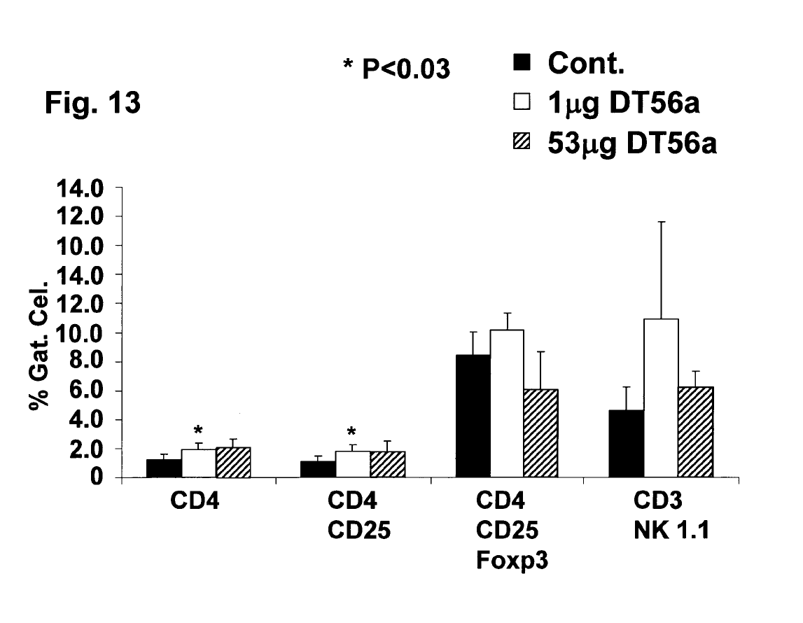

FIG. 13 shows that, following administration of 1 μg (low dose) of Femarelle for 6 weeks, CD25⁺ and CD4⁺CD25⁺ cells were significantly increased in the spleens of ob/ob mice (p<0.03). CD4⁺CD25⁺FOXP3⁺ and NK1.1 cells were also increased following administration of a low dose of Femarelle (p=NS). Serum TNF-αlevels were slightly elevated in mice treated with low and high doses of Femarelle (data not shown). The data suggest that, in the ob/ob model, oral administration of Femarelle promotes the recruitment of regulatory cells to damage sites and alleviates insulin resistance and the associated liver damage.

B. Oral Administration of Femarelle Alleviated Metabolic Syndrome in Mice Fed a High-Fat Diet

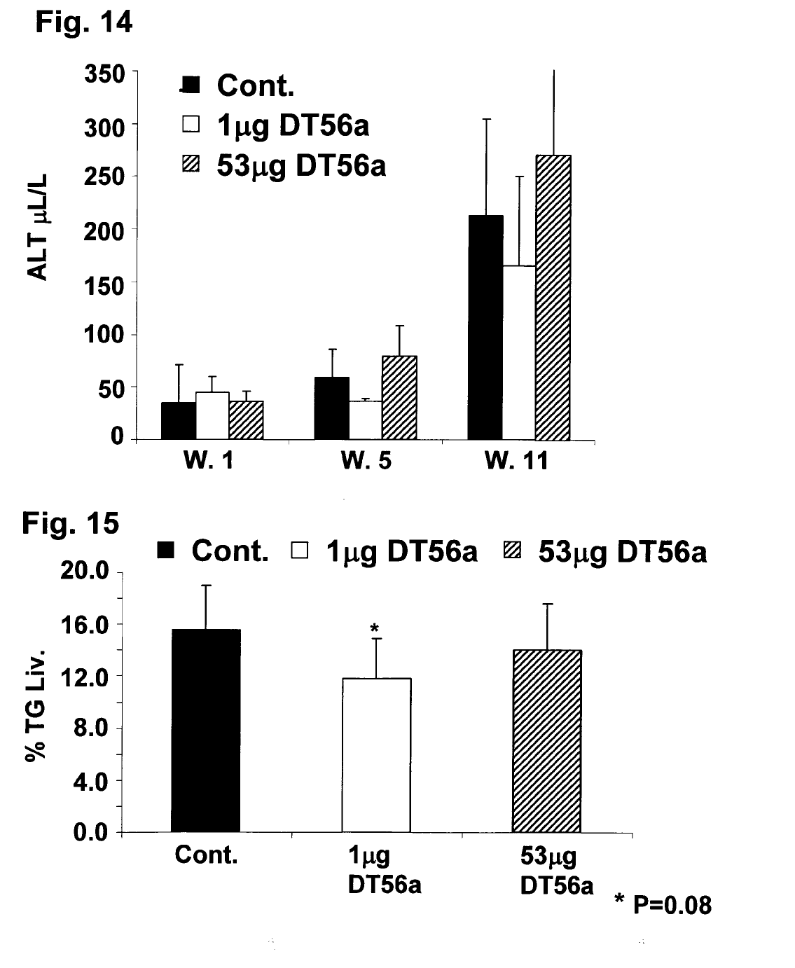

FIG. 14-19 show the effects of oral administration of either 1 μg (low dose) or 53 μg (high dose) Femarelle on the immune and metabolic parameters of mice that were fed a HFD for 11 weeks. FIG. 14 shows that a low dose of Femarelle was associated with a decrease in serum ALT levels. Similarly, FIG. 15 shows that the low-dose treatment was associated with a reduction of hepatic triglycerides levels (p=0.08).

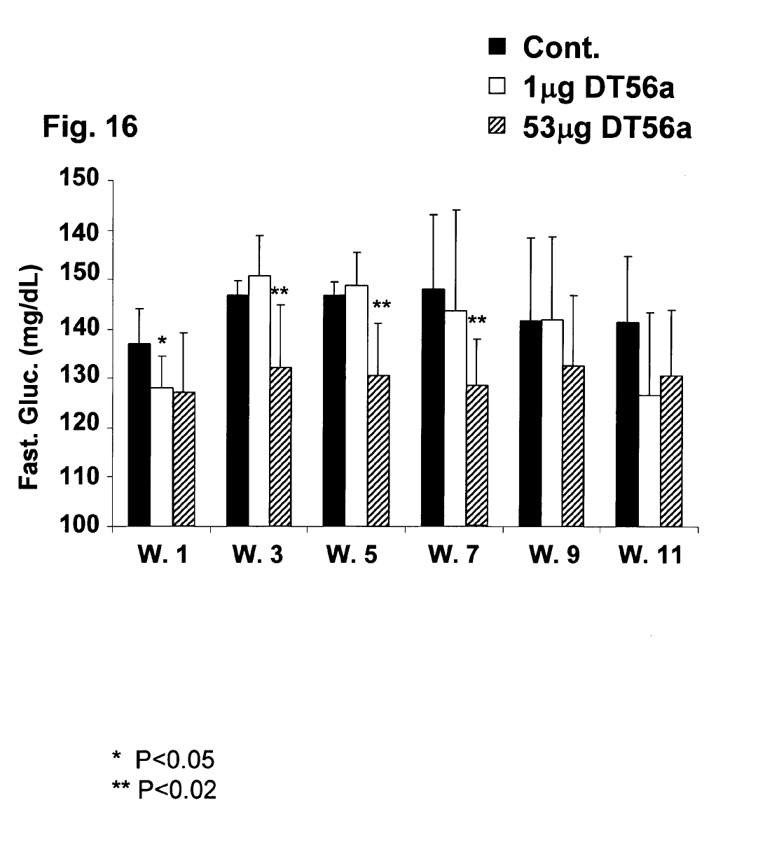

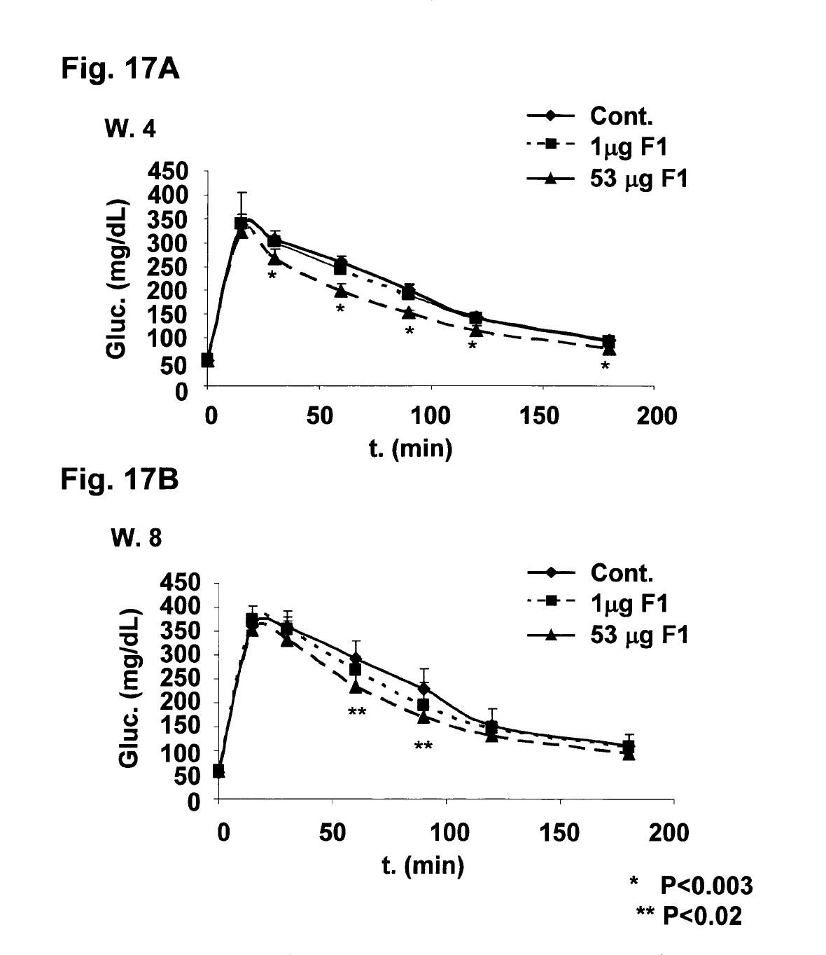

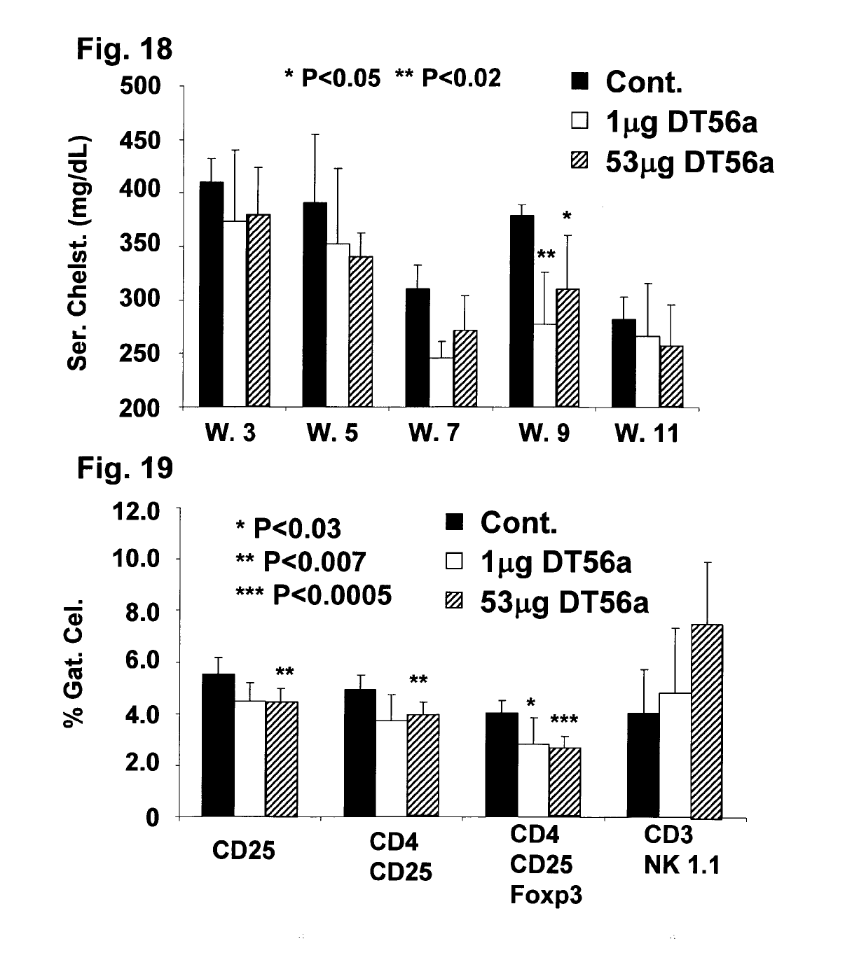

Similar to its effects in ob/ob mice, Femarelle improved insulin resistance in the HFD model. FIG. 16 shows a significant decrease in fasting blood glucose levels in HFD mice treated with high dose of Femarelle. This effect was first observed during the third week of the study (p<0.02). FIG. 17 shows the results of GTTs following 4 (FIG. 17A) and 8 (FIG. 17B) weeks of treatment. The GTT results demonstrate a significant effect of a high dose of Femarelle on lowering glucose levels in as little as 30 min, and this effect continued throughout the test. Similarly, a beneficial effect on serum cholesterol levels was noted in treated mice (FIG. 18). A significant decrease was noted in week 9 using both dosages. Serum triglyceride levels remained unchanged after treatment with Femarelle (data not shown). The beneficial effects of Femarelle in the HFD model were independent of changes in body weight (data not shown).

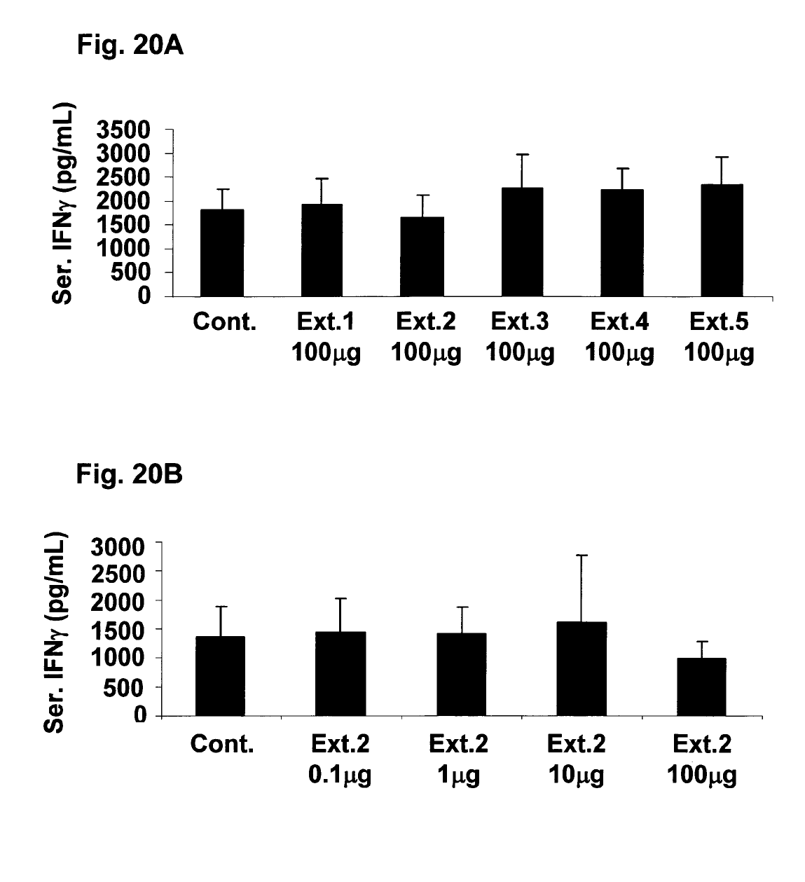

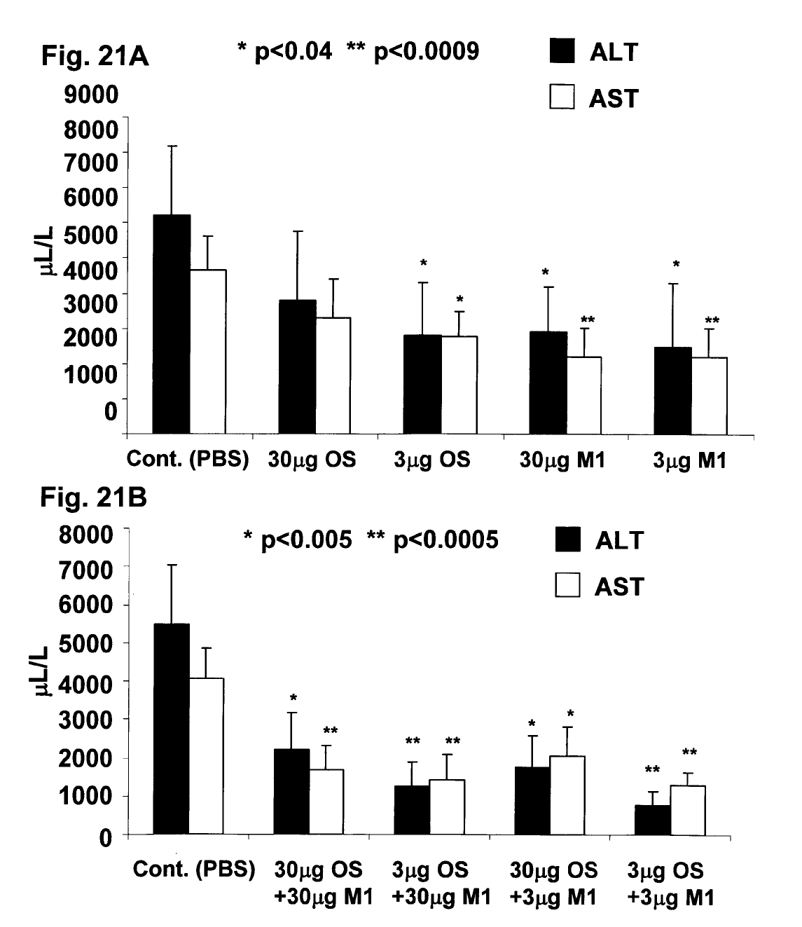

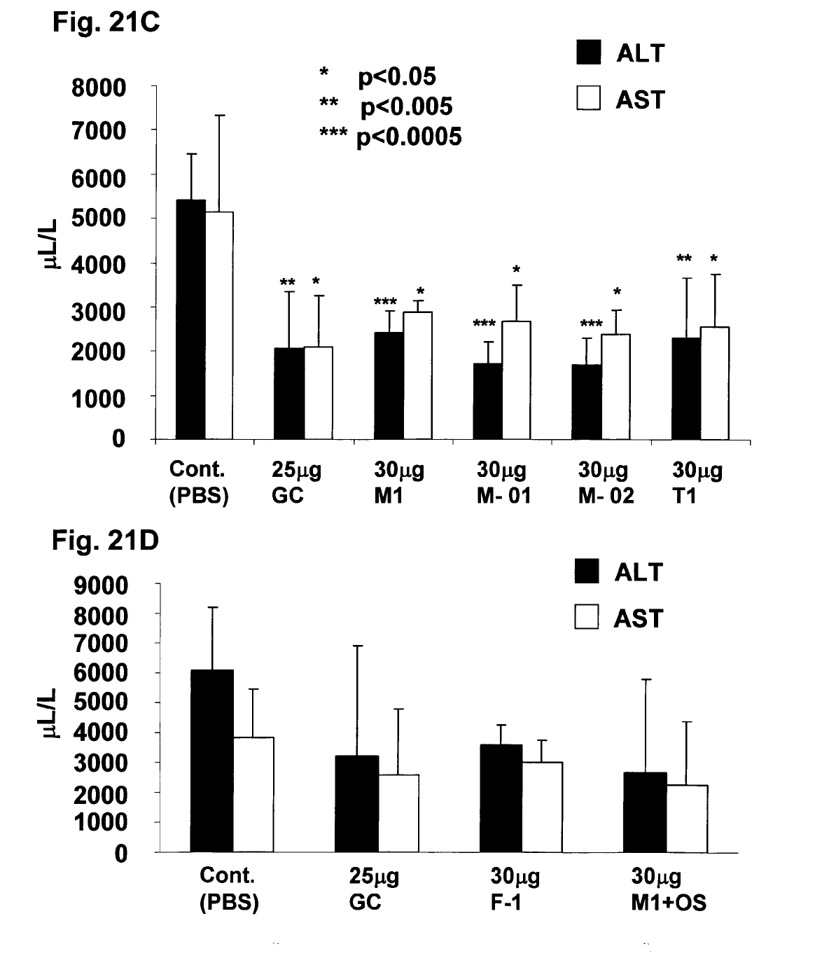

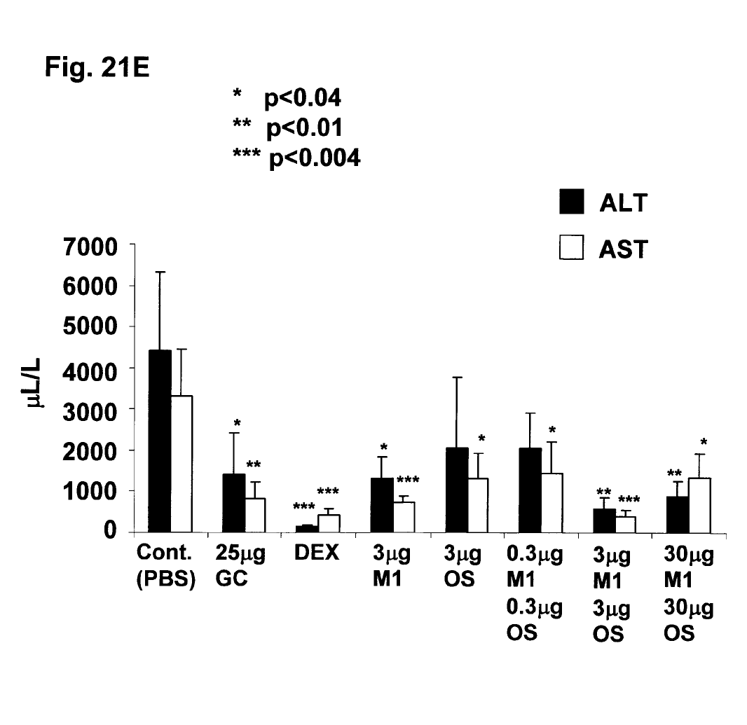

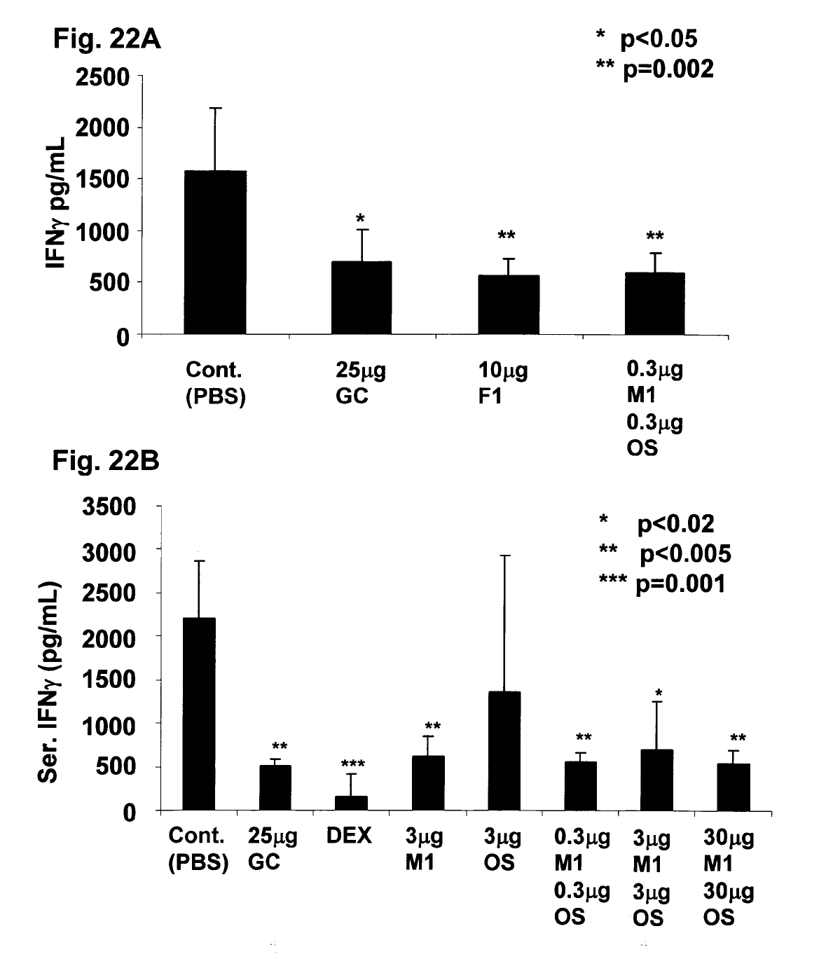

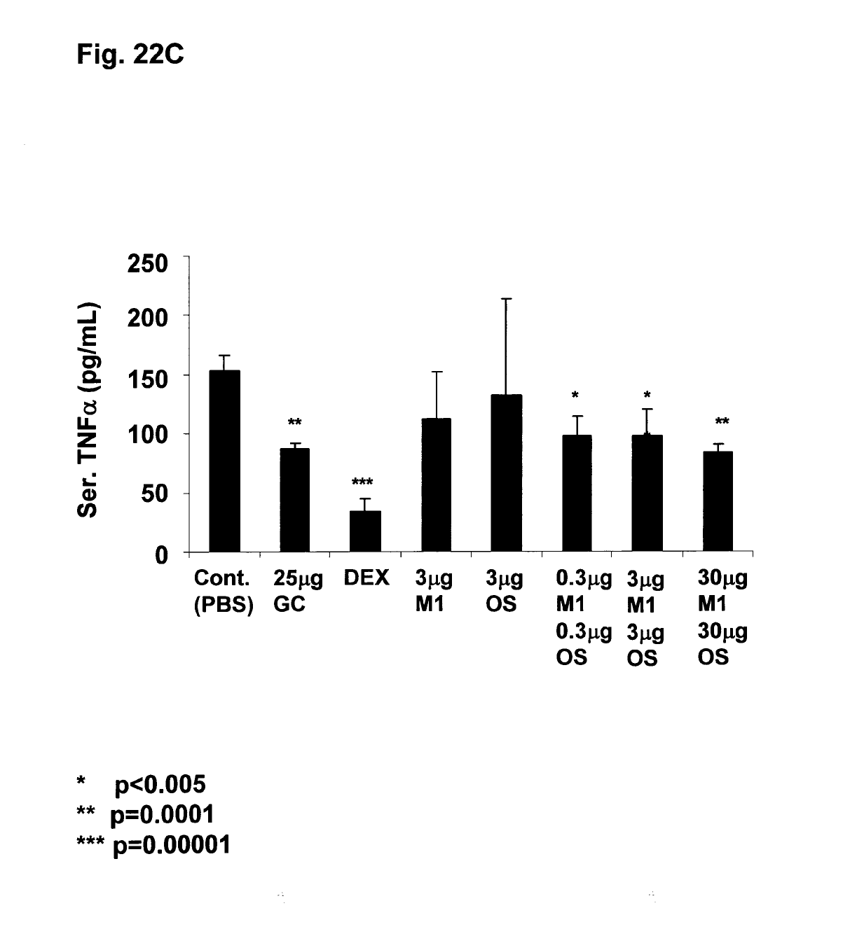

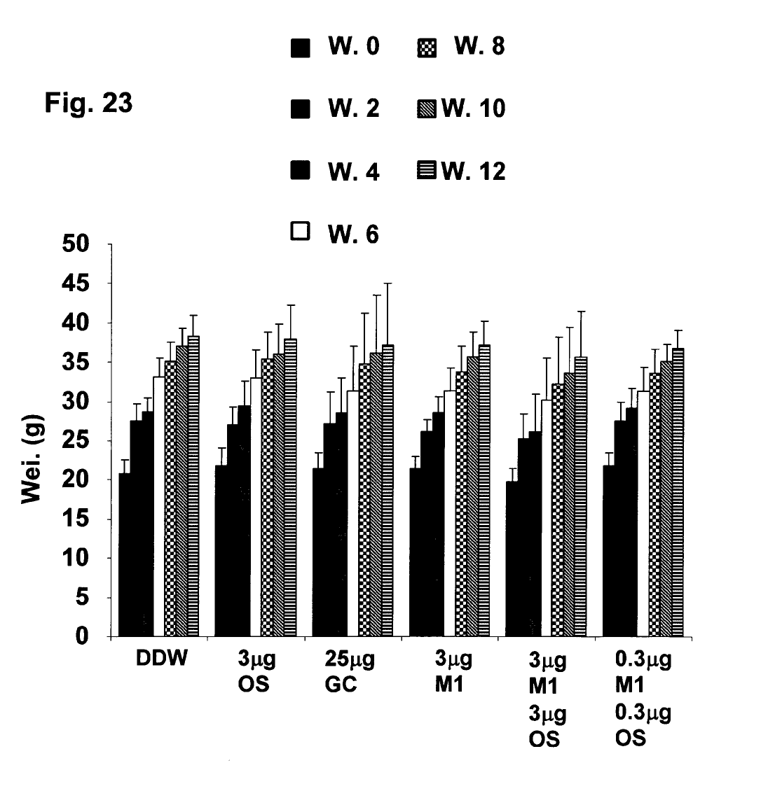

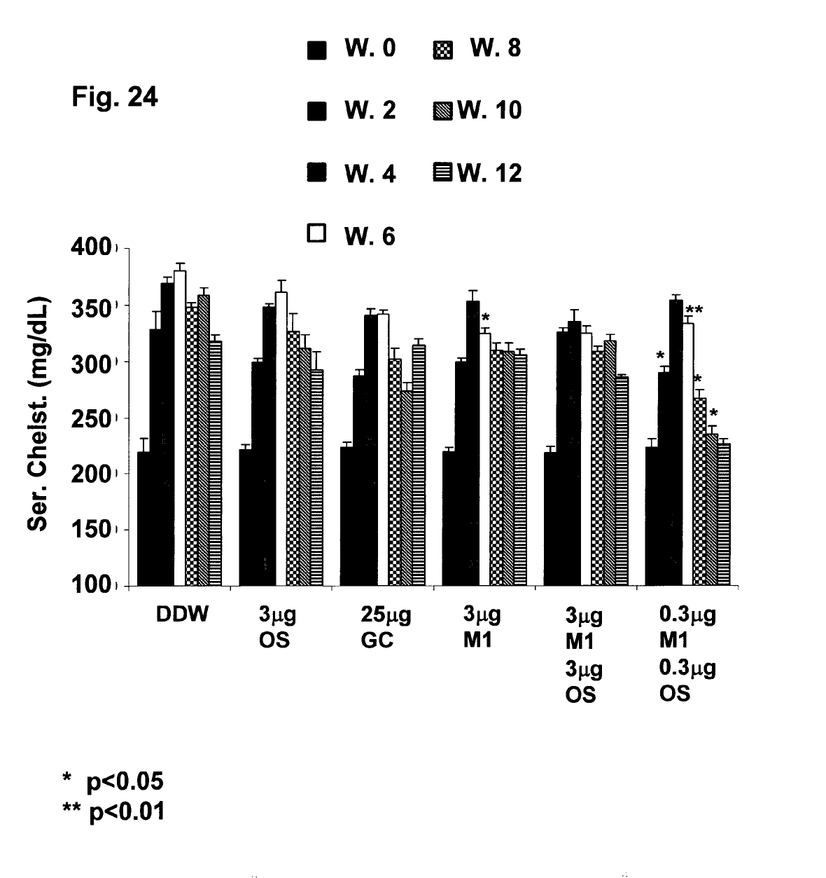

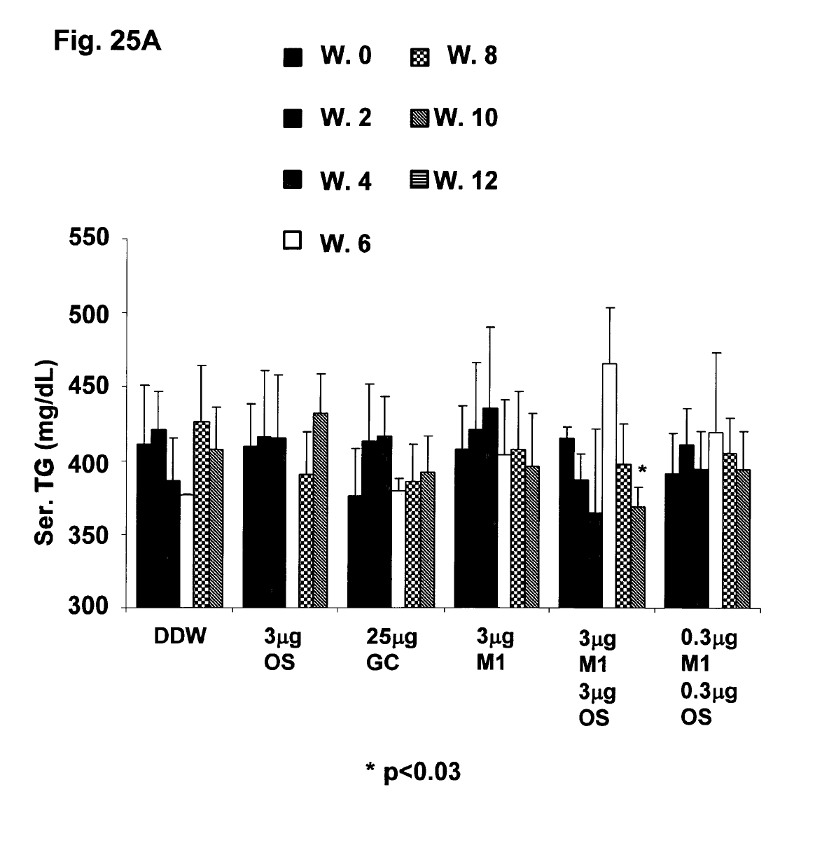

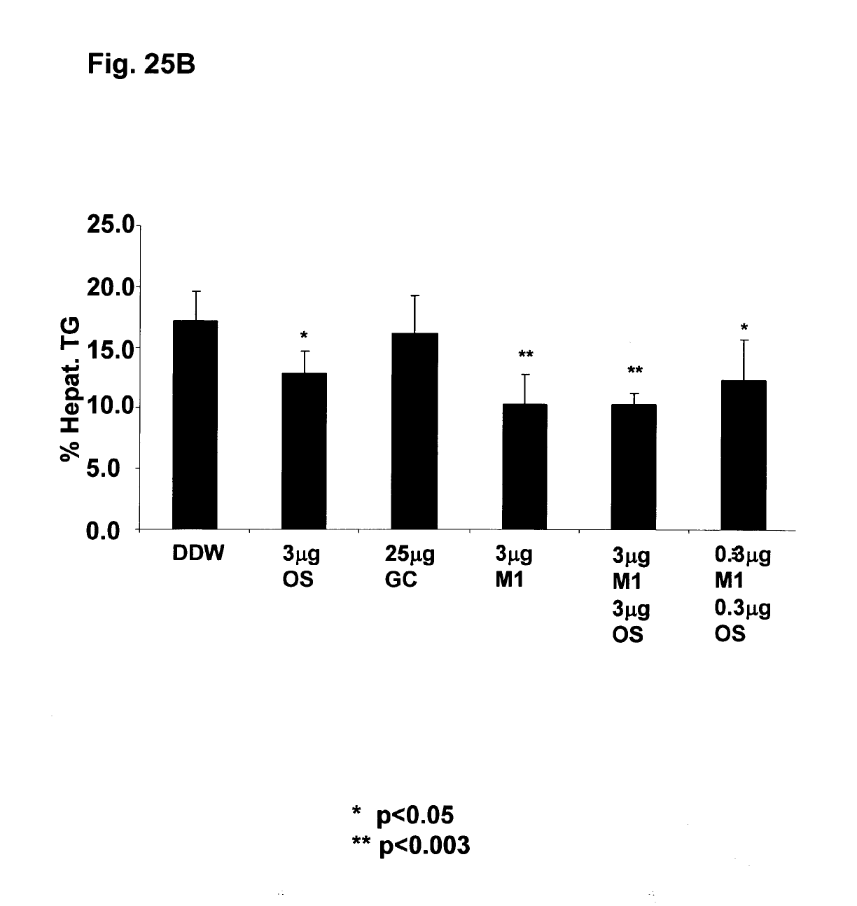

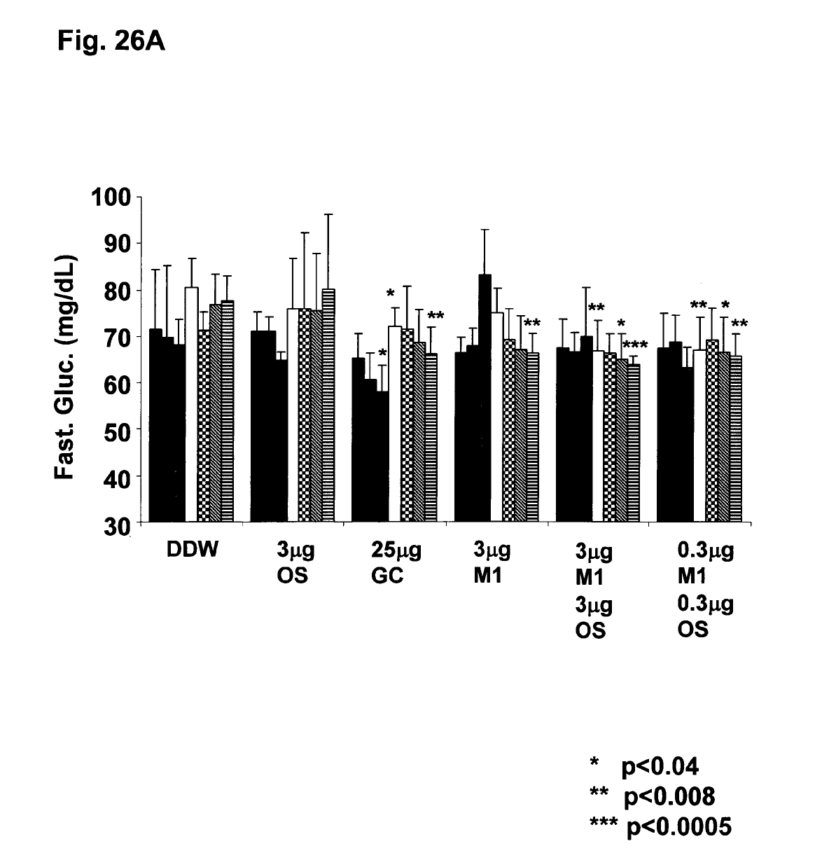

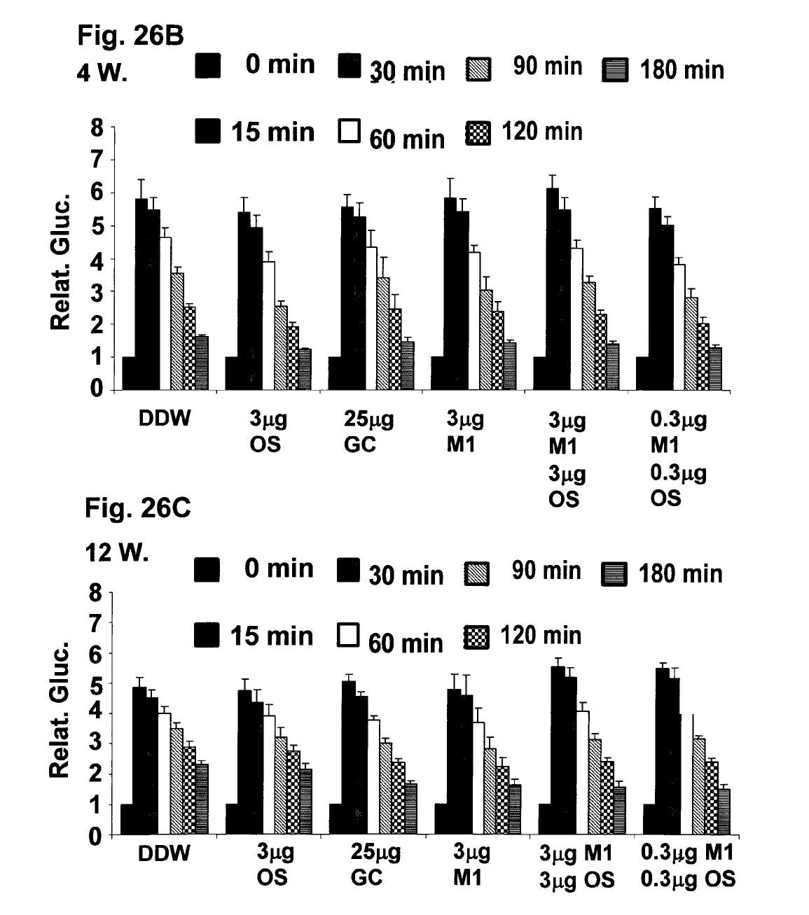

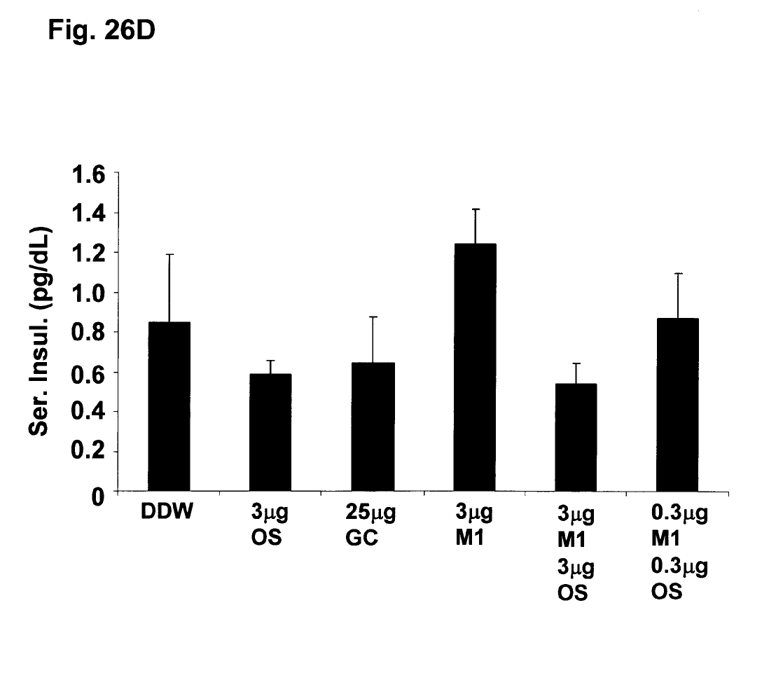

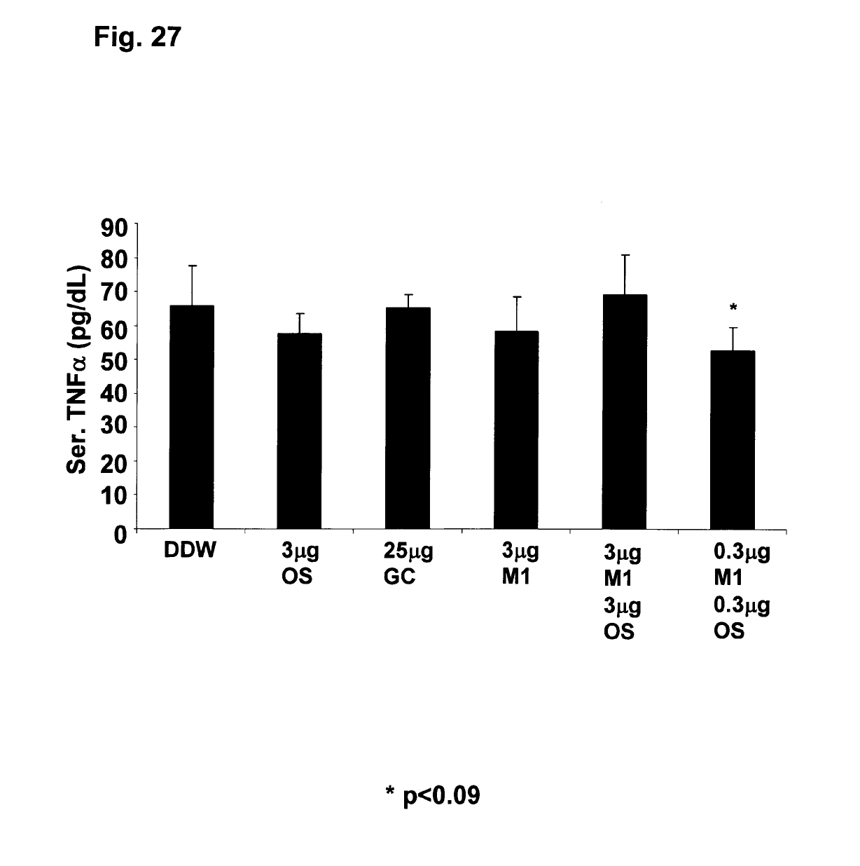

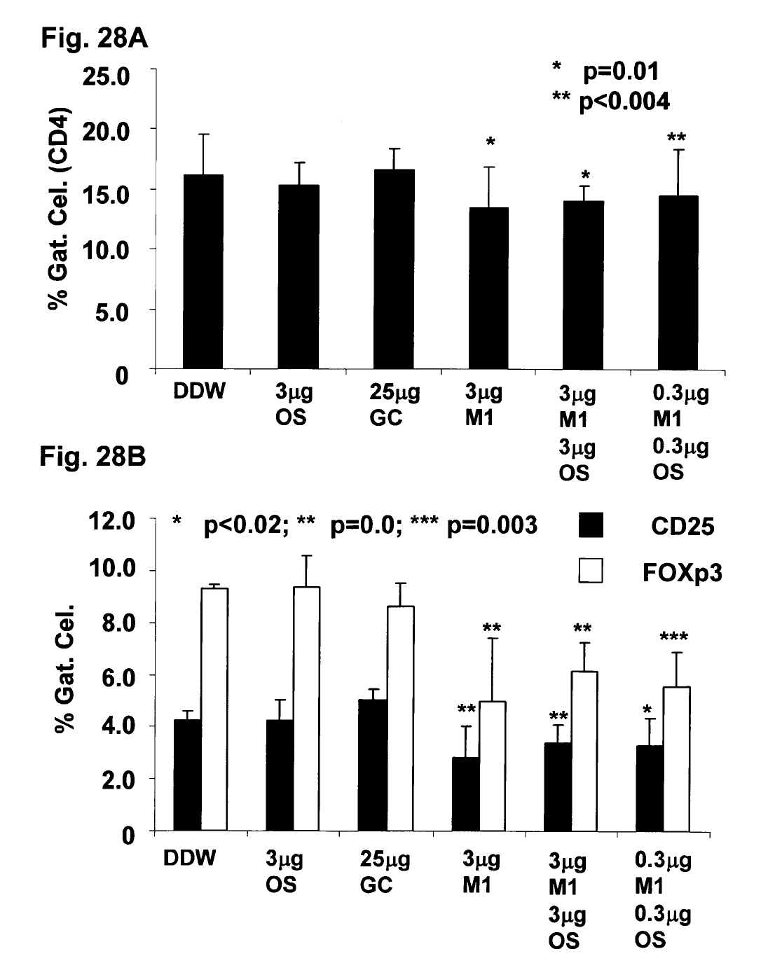

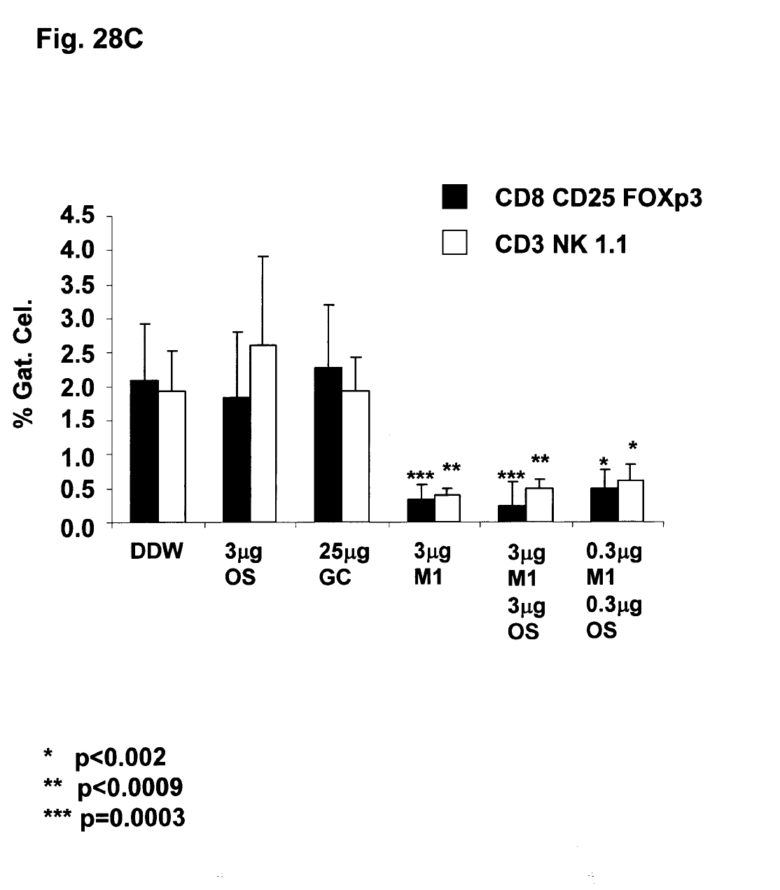

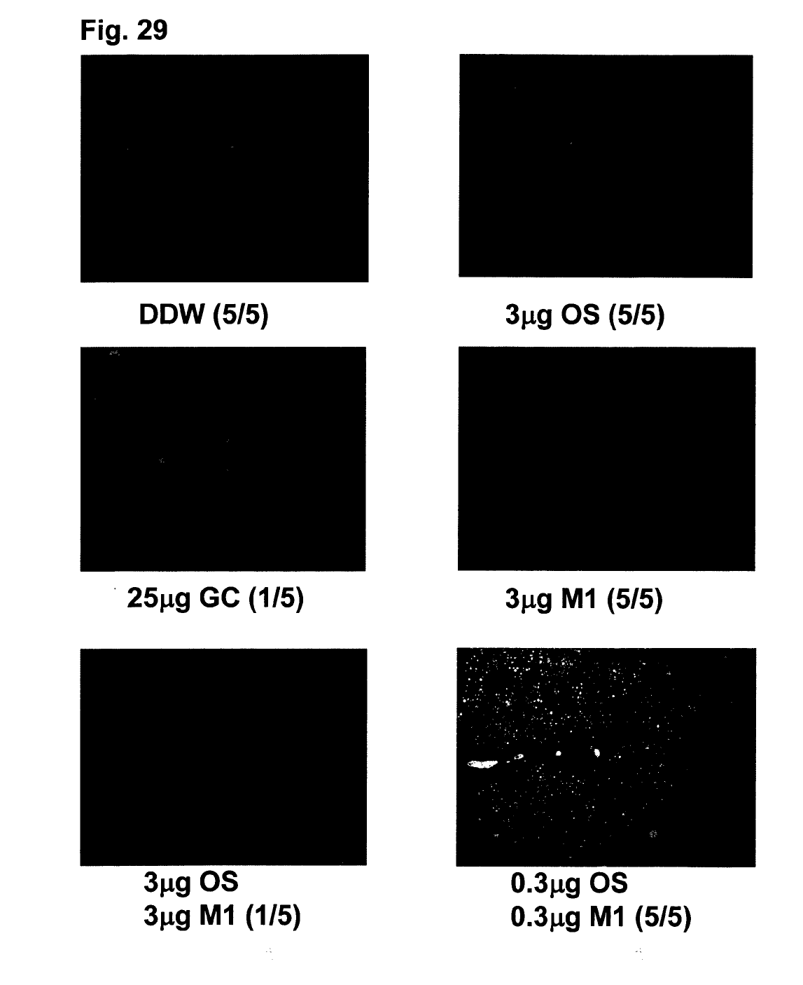

FIG. 19 shows a significant decrease in CD4⁺CD25⁺FOXP3 cells in the spleens of HFD mice treated with low dose of Femarelle (P<0.03). A more significant decrease in these cells was observed in animals treated with a high dose of Femarelle (P<0.0005). Furthermore, the mice treated with a high dose of Femarelle exhibited a significant decrease in the CD25⁺ and CD4⁺CD25⁺ subsets of lymphocytes. CD3⁺NK1.1⁺ cell populations were also elevated in the spleens of Femarelle-treated mice (P<0.03 for the high dose).

### Example 4

Preparation of an Optimized Femarelle Extract

Femarelle is marketed for use in the treatment of menopausal syndrome and bone loss via its effect as an estrogen receptor binding, and its immune modulatory effects, its hepato-protective effect and effect on the metabolic syndrome were previously described by the inventors. The inventors believe that the marketed Femarelle extraction/formulation may be optimized by further extraction steps. To achieve verify this goalhypothesis, different Femarelle extracts were analyzed.

Five extractions of Femarelle were prepared, each by a different solvent. More specifically, approximately 1.2 gr of Femarelle were extracted with 10 ml of solvent as described in Table 1, ultrasonicated for 30 minutes and incubated overnight. Extracts were filtered with filter paper No. 41 (Whatman) and evaporated using rotary evaporator (rotavapore). The weights and solvents of the different samples are presented in Table 1 below:

TABLE 1
Femarelle extracts - solvents and lyophilized weights

| | Dry weight in g | | Weight in g |
|---|---|---|---|
| # | (before extraction) | Solvent | (after extraction) |
| 1 | 1.2172 | H₂0 (water) | 0.0482 |
| 2 | 1.3264 | EtOH | 0.0703 |
| 3 | 1.2206 | Isopropanol | 0.1368 |
| 4 | 1.2319 | Acetone | 0.0407 |
| 5 | 1.2466 | 50% EtOH | 0.0251 |

Each one of extracts #1-#5 was then reconstituted as shown in Table 2:

TABLE 2
Reconstitution of Femarelle extracts

| | | Extract weight | Stock dissolved | | Stock |
|---|---|---|---|---|---|
| # | Solvent | after drying | in: | | Concentration |
| 1 | water | 48.2 mg | 482 | μl | 100 mg/ml |
| 2 | 30% C:E in 70% | 70.3 mg | 703 | μl | 100 mg/ml |
| | water | | | | |
| 3 | 30% C:E in 70% | 136.8 mg | 1.368 | ml | 100 mg/ml |
| | water | | | | |
| 4 | 30% C:E in 70% | 40.7 mg | 407 | μl | 100 mg/ml |
| | water | | | | |
| 5 | 30% C:E in 70% | 25.1 mg | 251 | μl | 100 mg/ml |
| | water | | | | |

C:E—Cremophor:Ethanol (C:E) in 1:1 ratio (v/v).

The dissolved extracts (stocks) were then diluted with DDW or 30% C:E in 70% water, to obtain a final concentration of 3.3 mg/ml and a final volume of 30 μl (yielding a total of 100 μg), as shown in Table 3:

TABLE 3
Dilution of Femarelle extract stocks

| | | Dilution | Volume | | Concen- | |
|---|---|---|---|---|---|---|
| # | Stock | ratio | (stock) | Solvent | tration | Total |
| 1 | 100 mg/ml | 1:33 | 30 μl | Water, 970 μl | 3.3 mg/ml | 100 μg |
| 2 | 100 mg/ml | 1:33 | 30 μl | C:E in water, | 3.3 mg/ml | 100 μg |
| | | | | 970 μl | | |
| 3 | 100 mg/ml | 1:33 | 30 μl | C:E in water, | 3.3 mg/ml | 100 μg |
| | | | | 970 μl | | |
| 4 | 100 mg/ml | 1:33 | 30 μl | C:E in water, | 3.3 mg/ml | 100 μg |
| | | | | 970 μl | | |
| 5 | 100 mg/ml | 1:33 | 30 μl | C:E in water, | 3.3 mg/ml | 100 μg |
| | | | | 970 μl | | |

Six groups of 4-5 11-12 weeks old male C57BL/6 mice were treated per os for three days with extracts as show in Table 4:

TABLE 4
Mice groups treatments

| Group | N | Extract # | Femarelle | Volume |
|---|---|---|---|---|
| A | 5 | — | — | 30 μl C:E in 70% water |
| | | | | (control) |
| B | 5 | 1 | yes | 30 μl |
| C | 5 | 2 | yes | 30 μl |
| D | 5 | 3 | yes | 30 μl |
| E | 5 | 4 | yes | 30 μl |
| F | 5 | 5 | yes | 30 μl |

The mice were injected i.v. (tail vein) 500 μg of ConA, (20 mg/kg body weight) and sacrificed 14 h later. After sacrifice measurements of serum IFN-γ was carried out using ELISA

FIG. 20A shows that extract #2 (EtOH extract reconstituted in 30 μl C:E in 70% water) effectively inhibited the ConA-induced serum IFN-γ increase. FIG. 10B shows that the 100 μg dose of extract #2 was the most efficient.

### Example 5

Soybean Derived Extracts

Encouraged by the surprising hepato-protective effect of Femarelle, the inventors next examined other soybean extracts. Five different soybean extracts were assessed for their capacity to induce beneficial anti-diabetic and anti-inflammatory effects. Extract OS comprises all lipids that are dissolved in hexane. M1 comprises 50% of dry matter, of which 60% is sucrose and the rest is raffinose and stachyose, 8% are proteins, 1% are isoflavins, 2% are saponins (2%). Minerals, lipids and other components are also present. M-01, M-02 and T1 are derived from M1 and have an unknown composition. The OS vehicle is Cremophor-Cl:Ethanol (C:E) in a 1:1 ratio (v/v) in 90% PBS. M1, M-01 and M-02 vehicle is DDW, and the T1 vehicle is PBS. GC is a natural β-glycolipid. It was prepared as an emulsion in Cremophor-Cl:Ethanol (C:E) in a 1:1 ratio (v/v) in 70% PBS.

### Example 6

Effects of Different Soybean-Derived Extracts on ConA-Induced Hepatotoxicity

To assess different soybean extracts effects on hepatotoxicity, 11-12 weeks old male C57BL/6 mice (4-6 per group) were administered the indicated amount of soybean extract selected from OS, M1, M-01, M-02, T1 as well as F-1 and GC per os each day for three days prior to ConA injection. As a positive control, 0.35 mg dexamethazone (Dex) were administered. In all experiments 5 mg (20 mg/kg body weight) ConA were injected i.v. (tail vein) to all mice.

Fourteen hours after administration of ConA, the mice were sacrificed and blood was cardially withdrawn and separated to serum and plasma. Serum ALT and AST activities were determined and serum levels of IFN-γ and TNF-α were measured.

As can be seen in FIG. 21A, both the M1 and OS extracts are hepatoprotective, reducing AST and ALT activity release, and a combination of the two extracts (especially 3 μg OS and 3 μM1) appears to be even more effective, as shown in FIG. 21B. FIG. 21C shows that extract M-01 and M-02 are similarly potent and effectively prevent the release of ALT activity. Interestingly, at the same time, these extracts are less effective in preventing the elevation of AST activity than GC, which serves as a positive (hepatoprotective) control. T-1 is also hepatoprotective. FIG. 21D compares the hepatoprotective action of the soy extracts combination 30 μM1 and 30 μg OS to the known effective extracts GC and F-1 (Femarelle). Surprisingly, the M1/OS mixture hepatoprotection provided superior results compared to the positive controls GC and F-1.

FIG. 21E shows that the 3 μg M1/3 μg OS mixture is as potent as the positive control treatment dexamethasone (DEX). FIG. 21E also demonstrates that the hepatoprotective activity of the extracts is very much dependent on the specific dosage of the combination of M1 and OS.

With respect to serum IFN-γ, a low-dose M1/OS mixture (0.3 μg each) is as effective as F-1 and more effective than GC, as illustrated in FIG. 22A.

FIG. 22B depicts the hepatoprotective effect exerted by different M1/OS mixtures as compared to M1 and OS separately, GC and dexamethasone, as reflected by serum IFN-γ. Although dexamethasone displays the most robust protection, the different mixtures, but not OS alone, provide good hepatoprotection.

Moreover, a clear reduction in serum TNF-α was demonstrated when different doses and combinations of M1 and OS were used, as illustrated in FIG. 22C.

In summary, the M1 and OS extracts yield the most efficient hepatic protection observed by decreasing liver enzymes and serum IFN-γ and TNF-α. OS administered alone was less effective than its combination with M1. The combinations (1:1) of these two extracts, especially the 0.3 and 3 μg per mouse, were the most effective soybean extracts. Positive controls (GC and DEX) were effective and resulted in a significant decrease of the two assayed parameters.

These experiments suggest the possible use of the tested soybean extracts (especially M1, OS, and more specifically, their mixtures) for use as hepatoprotective agents, providing significant protection against inflammation in the liver and associated disorders.

### Example 7

Effects of Different Soybean Extracts in Obesity-Associated Liver Disorder

Obesity is strongly associated with nonalcoholic fatty liver disease (NAFLD). Fatty livers are unusually susceptible to injury induced by inflammatory stress. A well accepted animal model of fatty liver is induced by feeding a high fat diet (HFD) Animals fed with HFD have elevated leptin levels, similar to obese humans. Mice with diet-induced obesity are characterized by elevated serum lipid profile, increased hepatic triglycerides and immune system alterations.

A HFD obese mouse model was therefore used to assess the hepatoprotective action of the soybean extracts M1, OS and their combinations. Young (6-7 weeks old) male C57BL/6 mice (5 mice per experimental group) were fed HFD diet ad libitum for 12 weeks. The mice were administered the indicated soybean extracts or combinations per os 3 times a week for the duration of the experiment. After 12 weeks, the mice were sacrificed. During the experiment the mice weight, fasting glucose level, ALT, serum triglycerides (TGs) and cholesterol were measured every 2 weeks, and glucose tolerance test was carried out after the fourth and twelfth weeks. On sacrifice day, serum samples and livers were collected. Serum TNF-α and insulin were determined, hepatic TG were measured and a liver section stained with H&E was prepared and analyzed. A FACS analysis of splenic T regulatory and NKT cells was also performed. The experiment design is presented in Table 5.

TABLE 5
Experiment design of HFD mice model

| Group | Treatment | Administration | Sacrifice |
|---|---|---|---|
| A | DDW | PO | After 3 months |
| N = 5 | 30 μl/mouse | | |
| B | OS, 3 μg | PO | After 3 months |
| N = 5 | 30 μl/mouse | | |
| C | GC, 25 μg | PO | After 3 months |
| N = 5 | 30 μl/mouse | | |
| D | M1, 3 μg | PO | After 3 months |
| N = 5 | 30 μl/mouse | | |
| E | M1 + OS, 3 μg + 3 μg | PO | After 3 months |
| N = 5 | 30 ml + 30 ml/mouse | | |
| F | M1 + OS, 0.3 μg + 0.3 μg | PO | After 3 months |
| N = 5 | 30 ml + 30 ml/mouse | | |

(PO—per os).

As FIG. 23 shows, no difference was detected in body weight gain due to HFD between negative control (DDW-treated) mice to OS-, M1-, or GC-treated mice, nor was a difference detected in mice treated with combinations of OS and M1. Thus, measured differences in metabolic and immune parameters found between the different experiment groups do not originate from changes in mice weights.

A low-dose combination of 0.3 μg M1 and 0.3 μg OS proved the most effective in lowering cholesterol to normal values after 12 weeks of HFD, in contrast with the other soybean extracts and combinations, which failed to normalize cholesterol, as depicted in FIG. 24. Similarly, FIG. 25A shows that a medium-dose combination of 3 μg M1 and 3 μg OS was the only treatment capable of significantly lowering serum triglycerides levels after 12 weeks in HFD. Hepatic TG levels shown in FIG. 25B were significantly lower in 3 μg OS and 3 μM1 mixture and M1-treated mice as compared to control mice. A low dose (0.3 μg OS and 0.3 μM1) extract mixture OS prevented TG in increase due to HFD, but to a lesser extent, while GC alone had no clear effect.

As can be seen in FIG. 26A, the fasting glucose levels of mice treated with the M1/OS mixtures were significantly improved (lowered) as compared to control mice, approximately on par with glucose levels in mice treated with GC, and better than in mice treated with OS alone.

The GTT analysis of mice after 4 and 12 weeks in HFD, presented in FIGS. 26B and 26C, respectively, showed that all treatments slightly improved GTT results (lower GTT endpoint level) at week 4 compared to control mice. After 12 weeks, mice treated with M1/OS combinations, M1 alone or GC alone showed marked improvement as compared to control and OS-treated mice.

The fasting insulin levels illustrated in FIG. 26D were lowest in mice treated with the combination of 3 μg OS and 3 μM1, OS alone or GC. Surprisingly, a lower-dose M1/OS combination and M1 alone did not lower insulin levels as compared to control mice.

FIG. 27 shows that the lower-dose 0.3 μM1/0.3 μg OS combination was the only treatment which succeeded in inhibiting TNF-α rise during HFD in a statistically-significant manner.

A FACS analysis of revealed that M1, and both M1/OS mixture doses (0.3 Kg each and 3 μg each) inhibited the HFD-induced increase in splenic regulatory CD4⁺CD25⁺FOXp3⁺ T cell population, as depicted in FIG. 28A. This inhibition also applied to splenic CD25⁺ and FOXp3⁺ populations, separately (FIG. 28B). However, the most dramatic effect, presented in FIG. 28C, was the inhibition of HFD-induced splenic CD8⁺CD25⁺FOXp3⁺ and CD3⁺NK1.1 populations increase by M1, and both M1/OS mixture doses.

The beneficial metabolic and anti-inflammatory effects of the soy extracts were also evident upon a visual inspection of H&E stained liver sections from the different mice. Representative sections are shown in FIG. 29, where a striking improvement (lower liver lipid accumulation) may be seen in mice treated with both M1/OS mixture doses (0.3 μg each and 3 μg each) or GC.

In summary, the HFD model experiment demonstrated that none of extracts had any effect on body weight. The low dose M1/OS mixture (0.3 μg each) was efficient in most cases, showing significant effects as early as in week 2 in decreasing serum total cholesterol. The 3 μM1/3 μg OS combination yielded the most significant serum and hepatic TG decrease in HFD mice. Liver histology had clearly shown that GC and the combination of OS and M1 dramatically decreased liver-accumulated lipids. Fasting glucose levels were significantly lower in GC and M1/OS combinations-treated mice. GTT was improved in all treatments by week 4 and 12. Serum TNF-α as a marker for inflammation which accompanies fatty liver disease, was significantly decreased only by the 0.3 μg OS/0.3 μM1 extract combination. Changes in regulatory and NKT splenic cell populations were observed in M1 treated mice and in mice treated with the two combinations (0.3 and 3 μg of OS and M1). In all tested parameters, OS administered alone was less effective compared to its combination with M1. The combinations (1:1 ratio) of these two extracts, in the 0.3 μg and 3 μg per mouse, were the most effective extracts in all tested parameters. Positive control (GC) was effective but not in all tested parameters.

1. A method of treating, ameliorating preventing or delaying the onset of any one of a hepatic disorder, drug induced hepatic injury, the Metabolic Syndrome or an immune-related disorder in a subject in need thereof; said method comprises the step of administering to said subject a therapeutically effective amount of at least one soybean extract, or any composition or mixture comprising the same, wherein said soybean extract is any one of an enzymatic soybean extract, a hexane extract and an aqueous extract.

2. (canceled)

3. The method according to claim 1, wherein said soybean extract is Femarelle (DT56a) or any extract thereof.

4-5. (canceled)

6. The method according to claim 1, wherein said at least one soybean extract is an extract selected from the group consisting of M1, OS, M-01, M-02 and T1, or any derivative, or any mixture or combination thereof.

7. The method according to claim 1, wherein said hepatic disorder is any one of an immune-mediated hepatitis, non alcoholic fatty liver disease and a drug induced hepatic injury (DILI).

8. (canceled)

9. The method according to claim 1, wherein said method leads to at least one of decrease in the plasma level of alanine aminotransferase (ALT), decrease in the plasma level of aspartate aminotransferase (AST), decrease in the plasma level of IFN-γ, decrease in the plasma level of TNF-α, decrease in the plasma level of total cholesterol, decrease in the plasma level of triglycerides, decrease in the plasma level of glucose, decrease in insulin resistance, decrease in hepatic apoptosis, decrease in hepatic necrosis, decrease in hepatic lipid accumulation and modulation of the distribution of at least one of Tregs and NK T cells in a subject in need thereof.

10. A method according to claim 1 for treating, preventing, ameliorating, reducing or delaying the onset of acute or chronic toxic effect of an analgesic or an antipyretic drug or any type of liver insult selected from infectious metabolic, toxic, immune, or perfusion or blood flow related hepatic injury, in a subject in need thereof wherein said therapeutically effective amount of at least one soybean extract, or any composition or mixture comprising the same, is being administered before, simultaneously with, after or any combination thereof, administration of said analgesic or antipyretic drug to said subject and wherein said at least one soybean extract is any one of an enzymatic soybean extract, a hexane extract and an aqueous extract.

11. (canceled)

12. The method according to claim 10, wherein said soybean extract is Femarelle (DT56a) or any extract thereof.

13. The method according to claim 10, wherein said analgesic drug is acetaminophen (paracetamol).

14. The method according to claim 10, wherein said acute or chronic toxic effect of said drug is any one of a drug induced liver injury (DILI), drug-induced acute steatosis, cytotoxic hepatocellular injury, acute liver failure (ALF), reperfusion injury, ischemic liver disease and acute cholestatic injury.

15. A composition comprising a combination of at least two of:
a. at least one soybean extract;
b. at least one enzymatic soybean extract;
c. at least one hexane soybean extract;
d. at least one aqueous soybean extract; and
e. at least one additional therapeutic agent, wherein said therapeutic agent is an analgesic or antipyretic drug;
said composition optionally further comprising at least one pharmaceutically acceptable carrier, diluent, excipient and/or additive.

16. The composition according to claim 15, comprising a combination of at least one hexane soybean extract and at least one aqueous soybean extract, wherein said aqueous soybean extract is extract M1 and said hexane soybean extract is extract OS, said composition optionally further comprises an additional therapeutic agent.

17. (canceled)

18. The composition according to claim 15, wherein said composition is a pharmaceutical composition, nutraceutical composition, functional food, functional nutrition product, medical food, medical nutrition product or dietary supplement.

19. The composition according to claim 15, wherein said composition is a pharmaceutical composition for treating, ameliorating preventing or delaying the onset of any one of hepatic disorder, drug induced hepatic injury, the Metabolic Syndrome or an immune-related disorder in a subject in need thereof.

20. (canceled)

21. The composition according to claim 15, wherein said soybean extract is Femarelle (DT56a) or any extract thereof and wherein said analgesic or antipyretic drug is acetaminophen (paracetamol).

22. The composition according to claim 21, wherein said composition is a pharmaceutical composition for treating, preventing, ameliorating, reducing or delaying the onset of acute or chronic toxic effect of an analgesic or an antipyretic drug or any type of liver insult selected from infectious metabolic, toxic, immune, or perfusion or blood flow related hepatic injury in a subject in need thereof and wherein said acute or chronic toxic effect of said drug is any one of drug induced liver injury (DILI), drug-induced acute steatosis, cytotoxic hepatocellular injury, acute liver failure (ALF), reperfusion injury, ischemic liver disease and acute cholestatic injury.

23-31. (canceled)

32. A kit comprising:
a. at least two of:
i. at least one soybean extract, optionally in a pharmaceutical dosage form;
ii. at least one enzymatic soybean extract, optionally in a pharmaceutical dosage form;
iii. at least one hexane soybean extract, optionally in a pharmaceutical dosage form;
iv. at least one aqueous soybean extract, optionally in a pharmaceutical dosage form; and
v. at least one additional therapeutic agent, optionally in a pharmaceutical dosage form;
b. optionally, container means for containing said at least two dosage forms.

33. The kit according to claim 32, for preventing acute or chronic toxic effect of an analgesic or an antipyretic drug or any type of liver insult selected from infectious metabolic, toxic, immune, or perfusion or blood flow related hepatic injury, said kit comprising:
a. at least one soybean extract, optionally in a first pharmaceutical dosage form;
b. at least one additional therapeutic agent, wherein said agent is an analgesic or an antipyretic drug, optionally in a second pharmaceutical dosage form; and
c. optionally, container means for containing said first and second dosage forms.

34. The kit according to claim 33, wherein said soybean extract is Femarelle (DT56a) or any extract thereof and wherein said analgesic or antipyretic drug is acetaminophen (paracetamol).

35. The kit according to claim 32 for treating, ameliorating preventing or delaying the onset of any one of hepatic disorder, drug induced hepatic injury, the Metabolic Syndrome or an immune-related disorder in a subject in need thereof, wherein said kit comprising:
a. at least one aqueous soybean extract M1, optionally in a first pharmaceutical dosage form; and
b. at least one hexane soybean extract OS, optionally in a first pharmaceutical dosage form; and
c. optionally, container means for containing said first and second dosage forms.

36. A method for increasing the maximum amount of acetaminophen administered to a subject without exhibiting acetaminophen toxicity, comprising administering of an acetaminophen toxicity inhibiting amount of a soybean extract, any enzymatically processed product or derivatives thereof, or any composition or mixture comprising the same, before, simultaneously with, after or any combination thereof, administration of said acetaminophen to said subject.
